(19) United States
(12) Patent Application Publication
(10) Pub. No.: US 2009/0105266 A1
(43) Pub. Date: Apr. 23, 2009

(54) Organic compounds

(76) Inventors: Ralf Glatthar, Bad Sackingen (DE); David Carcache, Binningen (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/148,557

(22) Filed: Apr. 18, 2008

(30) Foreign Application Priority Data
Apr. 19, 2007 (GB) 07106537.9

(51) Int. Cl. A61K 31/498; C07D 235/02; A61K 31/4184; A61K 31/4439; C07D 401/02; C07D 401/14; A61K 31/444; A61K 31/4709

(52) U.S. Cl. 514/249; 548/304.4; 514/394; 514/338; 546/273.1; 546/256; 514/333; 514/314; 546/152; 544/353

(57) ABSTRACT

The present invention relates to novel benzimidazole derivatives, their preparation, their use as pharmaceuticals and pharmaceutical compositions containing them, wherein the compounds have the formula (I):

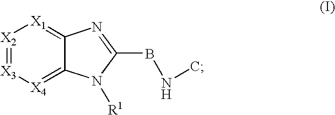

in which the substitutents are as defined in claim 1 and salts, solvates, hydrates and N-oxides thereof.

The present invention relates to novel benzimidazole derivatives, their preparation, their use as pharmaceuticals and pharmaceutical compositions containing them.

In a first aspect, the invention relates to a compound of formula (I) in free base or acid addition salt form;

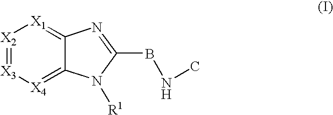

wherein:

X₁, X₂, X₃, X₄ each independently represent CR² or N, provided that at least two of X₁, X₂, X₃ and X₄ are CR²;

each R² independently is hydrogen, halogen, hydroxyl, nitro, cyano, formyl, carboxy, carboxamido, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino, (C₁₋₆alkoxycarbonyl)amino, C₁₋₆alkoxy, C₁₋₆alkoxycarbonyl, sulphonate, sulphate, phosphate, quartenary ammonium, phosphonate, guanidimium, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₃₋₁₂cycloalkyl, C₃₋₁₂halogencycloalkyl, C₁₋₆alkyl(C₃₋₁₂cycloalkyl), C₃₋₁₂cycloalkyl(C₁₋₆alkyl), C₃₋₁₂cycloalkyloxy, C₂₋₆alkenyl, C₂₋₆halogenalkenyl, C₂₋₆alkynyl or C₂₋₆halogenalkynyl;

R¹ is C₁₋₆alkyl, C₁₋₆halogenalkyl, C₃₋₁₂cycloalkyl, C₃₋₁₂halogencycloalkyl, C₁₋₆alkyl(C₃₋₁₂cycloalkyl) or C₃₋₁₂cycloalkyl(C₁₋₆alkyl);

or, when X₄ is CR², R¹, R² and the nitrogen and two carbon atoms, to which R¹ and R² are bound, may form together a 5- to 8-membered heterocyclic ring system, which may be aromatic or partially saturated and which may contain from 1 to 2 further hetero atoms selected from nitrogen, oxygen and sulfur, and wherein the heterocyclic ring system itself may be substituted once or more than once by R^{a};

each R^{a} independently is halogen, nitro, cyano, formyl, carboxy, carboxamido, hydroxyl, amino, (C₁₋₆alkyl)amino, di-(C₁₋₆alkyl)amino, (C₁₋₆alkoxycarbonyl)amino, C₁₋₆alkoxy, C₁₋₆alkoxycarbonyl, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₁₆alkyl), C₂₋₆alkenyl, C₂₋₆halogenalkenyl, C₂₋₆alkynyl or C₂₋₆halogenalkynyl;

B is

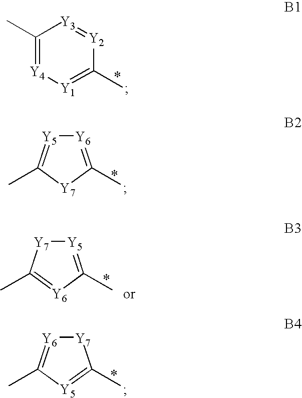

wherein the bond marked with the asterisk is attached to the group —NH—C;

Y₁, Y₂, Y₃ and Y₄ each independently represent CR³ or N, provided that at least one of Y₁, Y₂,

Y₃ and Y₄ is CR³;

Y₅ and Y₆ each independently represent CR³ or N, provided that at least one of Y₅ and Y₆ is CR³;

Y₇ is O, S or N(R^{3a});

each R³ independently is hydrogen, halogen, hydroxyl, nitro, cyano, formyl, carboxy, carboxamido, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino, (C₁₋₆alkoxycarbonyl)amino, C₁₋₆alkoxy, C₁₋₆alkoxycarbonyl, sulphonate, sulphate, phosphate, quartenary ammonium, phosphonate, guanidimium, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₃₋₁₂cycloalkyl, C₃₋₁₂halogencycloalkyl, C₁₋₆alkyl(C₃₋₁₂cycloalkyl), C₃₋₁₂cycloalkyl(C₁₋₆alkyl), C₃₋₁₂cycloalkyloxy, C₂₋₆alkenyl, C₂₋₆halogenalkenyl, C₂₋₆alkynyl or C₂₋₆halogenalkynyl;

R^{3a} is hydrogen, C₁₋₆alkyl, C₁₋₆halogenalkyl, C₃₋₁₂cycloalkyl, C₃₋₁₂halogencycloalkyl, C₁₋₆alkyl(C₃₋₁₂cycloalkyl) or C₃₋₁₂cycloalkyl(C₁₋₆alkyl);

C is a 5- to 12-membered aromatic ring system, which may be monocyclic or fused polycyclic, which may contain from 1 to 3 hetero atoms selected from nitrogen, oxygen and sulfur, and wherein the ring system itself may be substituted once or more than once by R^{b};

each R^{b} independently is halogen, hydroxyl, nitro, cyano, formyl, carboxy, carboxamido, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino, (C₁₋₆alkoxycarbonyl)amino, (C₁₋₆alkylcarbonyl)amino, C₁₋₆alkoxy, C₁₋₆alkoxycarbonyl, sulphonate, sulphate, phosphate, quartenary ammonium, phosphonate, guanidimium, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₃₋₁₂cycloalkyl, C₃₋₁₂halogencycloalkyl, C₁₋₆alkyl(C₃₋₁₂cycloalkyl), C₃₋₁₂cycloalkyl(C₁₋₆alkyl), C₃₋₁₂cycloalkyloxy, C₂₋₆alkenyl, C₂₋₆halogenalkenyl, C₂₋₆alkynyl or C₂₋₆halogenalkynyl; or two groups R^{b} bound to adjacent carbon atoms of the ring system together are a C₃₋₆alkandiyl group, wherein a carbon atom may be substituted by —O—, —S—, —N(R^{c})—, —C(═O)—, —C(═S)—, —C(═NR^{d})—, —S(═O)— or —SO₂—, and wherein the group may be substituted once or more than once by R^{e};

each R^{c}, R^{d} or R^{e} independently is halogen or C₁₋₆alkyl; or two groups R^{b} bound to adjacent carbon atoms of the ring system together are a group —O—(C(R^{f})₂)ₙ—O—;

each R^{f} independently is hydrogen, halogen or C₁₋₆alkyl; and

n is 1 or 2.

Preferred substituents, preferred ranges of numerical values or preferred ranges of the radicals present in compounds of the formula (I) and the corresponding intermediate compounds are defined below. The definition of the substituents applies to the end-products as well as to the corresponding intermediates. The definitions of the substituents may be combined at will, e.g. preferred substituents R¹ and particularly preferred substituents R².

In the present specification, the following definitions shall apply if no specific other definition is given:

“Halogen” preferably represents fluoro, chloro, bromo or iodo, more preferably represents fluoro, chloro or bromo and particularly preferably represents chloro.

“Alkyl” preferably represents a straight- or branched-chain C₁₋₆alkyl; more preferably represents a straight- or branched-chain C₁₋₄alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl or n-dodecyl; with particular preference given to methyl, ethyl, n-propyl, iso-propyl, n-butyl or iso-butyl.

“Alkandiyl” represents a straight-chain or branched-chain alkandiyl group bound by two different carbon atoms to the moiety, it preferably represents a straight-chain or branched-chain C₁₋₆alkandiyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl.

Each alkyl part of, for example, “alkoxy”, “alkoxyalkyl”, “alkoxycarbonyl”, “alkoxycarbonylalkyl” and “halogenalkyl” shall have the same meaning as described in the above-mentioned definition of “alkyl”.

“Alkenyl” represents a straight-chain or branched-chain C₂₋₆alkenyl group, for example, vinyl, allyl, 1-propenyl, isopropenyl, 2-butenyl, 2-pentenyl, 2-hexenyl, etc. and preferably represents C₂₋₄ alkenyl.

“Alkynyl” represents a straight-chain or branched-chain C₂₋₆alkynyl group, for example, ethenyl, propargyl, 1-propynyl, isopropenyl, 1-(2- or 3) butynyl, 1-(2- or 3) pentenyl, 1-(2- or 3) hexenyl, etc., preferably represents C₂₋₄alkynyl and particularly preferably represents ethynyl.

A substituent being substituted “once or more than once” is preferably substituted by one to three substituents.

“Cycloalkyl” contains 3 to 12 in-ring atoms and may be mono- or bicyclic. Preferred cycloalkyl groups contain 3 to 6 in-ring atoms. Exemplary cycloalkyls are cyclopropyl, cyclobutyl, cyclopentyl and cylclohexyl.

An “aromatic ring system” can be carbocyclic or heterocyclic and encompasses both “aryl” and “aromatic heterocyclyl”.

“Aryl” represents an aromatic hydrocarbon group, preferably a C₆₋₁₀ aromatic hydrocarbon group; for example phenyl, naphthyl, especially phenyl.

“Heterocyclic ring system” represents a saturated, partly saturated or aromatic ring system containing at least one hetero atom. Preferably, heterocycles consist of 3 to 12 ring atoms of which 1-3 ring atoms are hetero atoms selected from oxygen, sulfur or nitrogen. Heterocycles may be present as a single ring system or as bicyclic or tricyclic ring systems; preferably as single ring system or as benz-annelated ring system. Bicyclic or tricyclic ring systems may be formed by annelation of two or more rings, by a bridging atom, for example oxygen, sulfur, nitrogen or by a bridging group, e.g. alkandediyl or alkenediyl or be connected by a direct bond. Examples of heterocycles are: pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiaziolidine, isothiazole, istothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pterine, and the corresponding benz-annelated heterocycles, e.g. indole, isoindole, cumarine, cumaronecinoline, isochinoline, cinnoline and the like.

Examples of bivalent groups, which represent the group defined as “two groups R^{b} bound to adjacent carbon atoms of the ring system together are a group —O—(C(R^{f})₂)ₙ—O—” are —O—CH₂—O—, —O—CH₂—CH₂—O—, —O—CF₂—O— and —O—CH(CH₃)—O—.

As used herein, the term “variable components” refers to any of the moieties X₁, X₂, X₃, X₄, Y₁, Y₂, Y₃, Y₄, Y₅, Y₆, Y₇, Z₁, Z₂, Z₃, Z₄, Z₅, Z₆, Z₇, Z₈, Z₉, R¹, R², R², R^{2b}, R^{2c}, R^{2d}, R³, R³R⁴, R^{4a}, R^{4b}, R^{4c}, R⁵, R⁶, R^{a}, R^{b}, R^{c}, R^{d}, R^{e}, R^{f} and/or n present in the corresponding general formula.

Compounds of the present invention may exist in free or acid addition salt form. In this specification, unless otherwise indicated, language such as “compounds of formula (I)”, for example is to be understood as embracing the compounds in any form, for example free base or acid addition salt form. Salts which are unsuitable for pharmaceutical uses but which can be employed, for example, for the isolation or purification of free compounds of the present invention, such as picrates or perchlorates, are also included. For therapeutic use, only pharmaceutically acceptable salts or free compounds are employed (where applicable in the form of pharmaceutical preparations), and are therefore preferred.

Compounds of the present invention may exist as a N-oxide derivatives. All N-oxide derivatives are part of the present invention.

Tautomers can, e.g., be present in cases where amino or hydroxy, each with a least one bound hydrogen, are bound to carbon atoms that are bound to adjacent atoms by double bonds (e.g. keto-enol or imine-enamine tautomerism). All tautomers are part of the present invention.

On account of the asymmetrical carbon atom(s) that may be present in the compounds of the present invention and their salts, the compounds may exist in optically active form or in form of mixtures of optical isomers, e.g. in form of racemic mixtures or diastereomeric mixtures. All optical isomers and their mixtures, including the racemic mixtures, are part of the present invention.

In one aspect of the present invention, C is:

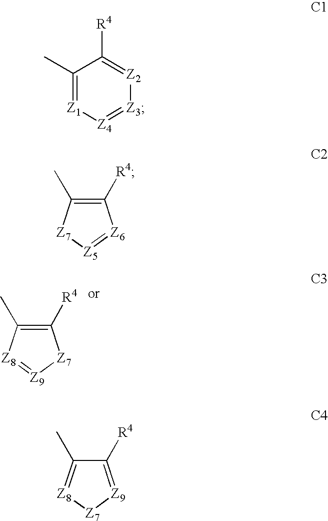

wherein

Z₁, Z₂, Z₃ and Z each independently represent CR⁴ or N, provided that at least two of Z₁, Z₂, Z₃ and Z are CR⁴; and

Z₅ and Z₆ each independently represent CR⁴ or N, provided that at least one of Z₅ and Z₆ is CR⁴;

Z₈ and Z₉ each independently represent CR⁴ or N, provided that at least one of Z₈ and Z₉ is CR⁴;

Z₇ is O, S or N(R^{4a});

each R⁴ individually represents hydrogen, halogen, hydroxyl, nitro, cyano, formyl, carboxy, carboxamido, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino, (C₁₋₆alkoxycarbonyl)amino, (C₁₋₆alkylcarbonyl)amino, C₁₋₆alkoxy, C₁₋₆alkoxycarbonyl, sulphonate, sulphate, phosphate, quartenary ammonium, phosphonate, guanidimium, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₃₋₁₂cycloalkyl, C₃₋₁₂halogencycloalkyl, C₁₋₆alkyl(C₃₋₁₂cycloalkyl), C₃₋₁₂cycloalkyl(C₁₋₆alkyl), C₃₋₁₂cycloalkyloxy, C₂₋₆alkenyl, C₂₋₆halogenalkenyl, C₂₋₆alkynyl or C₂₋₆halogenalkynyl;

R^{4a} is hydrogen, C₁₋₆alkyl, C₁₋₆halogenalkyl, C₃₋₁₂cycloalkyl, C₃₋₁₂halogencycloalkyl, C₁₋₆alkyl(C₃₋₁₂cycloalkyl) or C₃₋₁₂cycloalkyl(C₁₋₆alkyl);

or, when Z₂ and Z₃ are both CR⁴, these two R⁴ groups may, together with the two carbon atoms to which they are attached, form a 5- or 6-membered aryl or aromatic heterocyclic ring system, which may be substituted once or more than once by halogen, C₁₋₆alkyl or C₁₋₆halogenalkyl;

or, when Z₅ and Z₆ are both CR⁴, these two R⁴ groups may, together with the two carbon atoms to which they are attached, form a 5- or 6-membered aryl or aromatic heterocyclic ring system, which may be substituted once or more than once by halogen, C₁₋₆alkyl or C₁₋₆halogenalkyl. Preferably, the ring formed by Z₂ and Z₃ or by Z₅ and Z₆ is aromatic heterocyclyl. In one embodiment, said aromatic heterocyclyl is unsubstituted.

One embodiment of the present invention are compounds of formula (I)

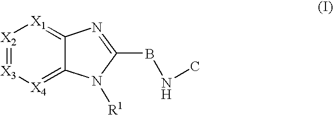

wherein:

X₁, X₂, X₃, X₄ each independently represent CR² or N, provided that at least two of X₁, X₂, X₃ and X₄ are CR²;

each R² independently is hydrogen, halogen, hydroxyl, nitro, cyano, formyl, carboxy, carboxamido, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino, (C₁₋₆alkoxycarbonyl)amino, C₁₋₆alkoxy, C₁₋₆alkoxycarbonyl, sulphonate, sulphate, phosphate, quartenary ammonium, phosphonate, guanidimium, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₃₋₁₂cycloalkyl, C₃₋₁₂halogencycloalkyl, C₁₋₆alkyl(C₃₋₁₂cycloalkyl), C₃₋₁₂cycloalkyl(C₁₋₆alkyl), C₃₋₁₂cycloalkyloxy, C₂₋₆alkenyl, C₂₋₆halogenalkenyl, C₂₋₆alkynyl or C₂₋₆halogenalkynyl;

R¹ is C₁₋₆alkyl, C₁₋₆halogenalkyl, C₃₋₁₂cycloalkyl, C₃₋₁₂halogencycloalkyl, C₁₋₆alkyl(C₃₋₁₂cycloalkyl) or C₃₋₁₂cycloalkyl(C₁₋₆alkyl);

or, when X₄ is CR², R¹, R² and the nitrogen and two carbon atoms, to which R¹ and R² are bound, may form together a 5- to 8-membered heterocyclic ring system, which may be aromatic or partially saturated and which may contain from 1 to 2 further hetero atoms selected from nitrogen, oxygen and sulfur, and wherein the heterocyclic ring system itself may be substituted once or more than once by R^{a};

each R^{a} independently is halogen, nitro, cyano, formyl, carboxy, carboxamido, hydroxyl, amino, (C₁₋₆alkyl)amino, di-(C₁₋₆alkyl)amino, (C₁₋₆alkoxycarbonyl)amino, C₁₋₆alkoxy, C₁₋₆alkoxycarbonyl, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₂₋₆alkenyl, C₂₋₆halogenalkenyl, C₂₋₆alkynyl or C₂₋₆halogenalkynyl;

B is

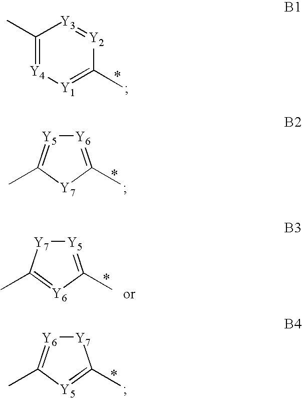

wherein the bond marked with the asterisk is attached to the group —NH—C;

Y₁, Y₂, Y₃ and Y₄ each independently represent CR³ or N, provided that at least one of Y₁, Y₂, Y₃ and Y₄ is CR³;

Y₅ and Y₆ each independently represent CR³ or N, provided that at least one of Y₅ and Y₆ is CR³;

Y₇ is O, S or N(R^{3a});

each R³ independently is hydrogen, halogen, hydroxyl, nitro, cyano, formyl, carboxy, carboxamido, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino, (C₁₋₆alkoxycarbonyl)amino, C₁₋₆alkoxy, C₁₋₆alkoxycarbonyl, sulphonate, sulphate, phosphate, quartenary ammonium, phosphonate, guanidimium, C₁₋₆alkyl, C₁₋₆halogenalkyl, C₁₋₆hydroxyalkyl, C₁-C₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₃₋₁₂cycloalkyl, C₃₋₁₂halogencycloalkyl, C₁₋₆alkyl(C₃₋₁₂cycloalkyl), C₃₋₁₂cycloalkyl(C₁₋₆alkyl), C₃₋₁₂cycloalkyloxy, C₂₋₆alkenyl, C₂₋₆halogenalkenyl, C₂₋₆alkynyl or C₂₋₆halogenalkynyl;

R^{3a} is hydrogen, C₁₋₆alkyl, C₁₋₆halogenalkyl, C₃₋₁₂cycloalkyl, C₃₋₁₂halogencycloalkyl, C₁₋₆alkyl(C₃₋₁₂cycloalkyl) or C₃₋₁₂cycloalkyl(C₁₋₆alkyl);

C is:

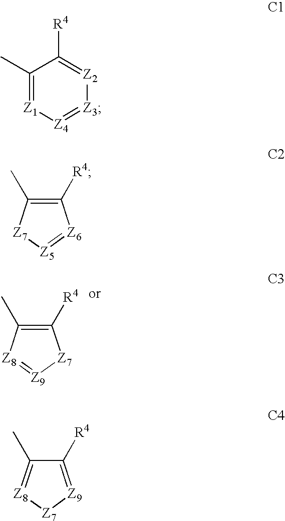

wherein

Z₁, Z₂, Z₃ and Z each independently represent CR⁴ or N, provided that at least two of Z₁, Z₂, Z₃ and Z are CR⁴; and

Z₅ and Z₆ each independently represent CR⁴ or N, provided that at least one of Z₅ and Z₆ is CR⁴;

Z₈ and Z₉ each independently represent CR⁴ or N, provided that at least one of Z₈ and Z₉ is CR⁴;

Z₇ is O, S or N(R^{4a});

each R⁴ individually represents hydrogen, halogen, hydroxyl, nitro, cyano, formyl, carboxy, carboxamido, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino, (C₁₋₆alkoxycarbonyl)amino, (C₁₋₆alkylcarbonyl)amino, C₁₋₆alkoxy, C₁₋₆alkoxycarbonyl, sulphonate, sulphate, phosphate, quartenary ammonium, phosphonate, guanidimium, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₃₋₁₂cycloalkyl, C₃₋₁₂halogencycloalkyl, C₁₋₆alkyl(C₃₋₁₂cycloalkyl), C₃₋₁₂cycloalkyl(C₁₋₆alkyl), C₃₋₁₂cycloalkyloxy, C₂₋₆alkenyl, C₂₋₆halogenalkenyl, C₂₋₆alkynyl or C₂₋₆halogenalkynyl;

R^{4a} is hydrogen, C₁₋₆alkyl, C₁₋₆halogenalkyl, C₃₋₁₂cycloalkyl, C₃₋₁₂halogencycloalkyl, C₁₋₆alkyl(C₃₋₁₂cycloalkyl) or C₃₋₁₂cycloalkyl(C₁₋₆alkyl);

or, when Z₂ and Z₃ are both CR⁴, these two R⁴ groups may, together with the two carbon atoms to which they are attached, form a 5- or 6-membered aryl or aromatic heterocyclic ring system, which may be substituted once or more than once by halogen, C₁₋₆alkyl or C₁₋₆halogenalkyl;

or, when Z₅ and Z₆ are both CR⁴, these two R⁴ groups may, together with the two carbon atoms to which they are attached, form a 5- or 6-membered aryl or aromatic heterocyclic ring system, which may be substituted once or more than once by halogen, C₁₋₆alkyl or C₁₋₆halogenalkyl. Preferably, the ring formed by Z₂ and Z₃ or by Z₅ and Z₆ is aromatic heterocyclyl. In one embodiment, said aromatic heterocyclyl is unsubstituted.

In one embodiment of the present invention, B is B1.

In one embodiment of the present invention, B is selected from B2, B3 and B4.

In one embodiment of the present invention, B is B2.

In one embodiment of the present invention, B is B3.

In one embodiment of the present invention, B is B4.

In one embodiment of the present invention, C is C1.

In one embodiment of the present invention, C is selected from C2, C3 and C4.

In one embodiment of the present invention, C is C2.

In one embodiment of the present invention, C is C3.

In one embodiment of the present invention, C is C4.

In one embodiment of the present invention, there are provided compounds of the formula (II):

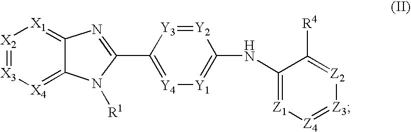

wherein:

X₁, X₂, X₃, X₄ each independently represent CR² or N, provided that at least two of X₁, X₂, X₃ and X₄ are CR²;

R² is hydrogen, halogen, hydroxy, C₁₋₆alkyl, C₁₋₆halogenalkyl, C₃₋₁₂cycloalkyl, amino, C₁₋₆alkoxy, C₃₋₁₂cycloalkyloxy, sulphonate, sulphate, phosphate, quartenary ammonium, phosphonate, guanidimium or cyano;

R¹ is C₁₋₆alkyl, C₃₋₁₂cycloalkyl or C₃₋₁₂cycloalkyl-C₁₋₆alkyl;

or, when X₄ is CR², R¹, R² and the nitrogen and two carbon atoms, to which R¹ and R² are bound, may form together a 5- to 8-membered heterocyclic ring system, which may be aromatic or partially saturated and which may contain from 1 to 2 further hetero atoms selected from nitrogen, oxygen and sulfur, and wherein the heterocyclic ring system itself is unsubstituted;

Y₁, Y₂, Y₃ and Y₄ each independently represent CR³ or N, provided that at least one of Y₁, Y₂, Y₃ and Y₄ is CR³;

R³ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino or cyano;

Z₁, Z₂, Z₃ and Z₄ each independently represent CR⁴ or N, provided that at least two of Z₁, Z₂, Z₃ and Z are CR⁴; and

R⁴ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino or di(C₃₋₁₂cycloalkyl)amino;

or, when Z₂ and Z₃ are both CR⁴, these two R⁴ groups may, together with the two carbon atoms to which they are attached, form a 5- or 6-membered aryl or aromatic heterocyclic ring system, which is unsubstituted; in one embodiment, said ring system is a 6-membered aromatic heterocyclic ring system.

In a further preferred embodiment of said embodiment,

X₁, X₂, X₃, X₄ each independently represent CR² or N, provided that at least two of X₁, X₂, X₃ and X₄ are CR²;

R² is hydrogen, halogen, C₁₋₆alkyl, C₃₋₆cycloalkyl;

R¹ is C₁₋₆alkyl or C₃₋₆cycloalkyl-C₁₋₆alkyl;

Y₁, Y₂, Y₃ and Y₄ each independently represent CR³ or N, provided that at least one of Y₁, Y₂, Y₃ and Y₄ is CR³;

R³ represents hydrogen or halogen;

Z₁, Z₂, Z₃ and Z₄ each independently represent CR⁴ or N, provided that at least two of Z₁, Z₂, Z₃ and 4 are CR⁴; and

R⁴ represents hydrogen, halogen, C₁₋₆alkyl, C₃₋₆cycloalkyl, C₁₋₆alkoxy, C₃₋₆cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino or di(C₃₋₁₂cycloalkyl)amino.

Herein, compounds of the formula (I), (II), (III) or (IV) can be also represented by formula (I′):

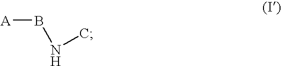

wherein ring A represents the group:

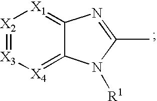

and the variable components as well as rings B and C are as described herein.

In one embodiment of the present invention, X₁ and X₂ are CH and X₃ and X₄ are CR².

In one embodiment of the present invention, said ring A has the formula (A1):

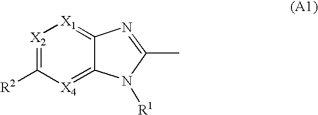

wherein the variable components are as herein described.

In a second embodiment, ring A has the formula (A2):

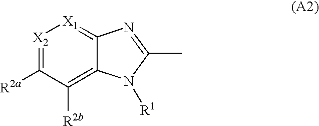

wherein each R^{2a} and R^{2b} are independently selected from hydrogen, halogen, hydroxy, C₁₋₆alkyl, C₁₋₆halogenalkyl, C₃₋₁₂cycloalkyl, amino, C₁₋₆alkoxy, C₃₋₁₂cycloalkyloxy, sulphonate, sulphate, phosphate, quartenary ammonium, phosphonate, guanidimium or cyano; and the remaining variable components are as herein described.

In a third embodiment, ring A has the formula (A3):

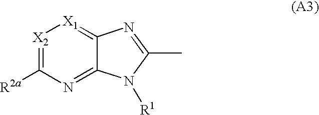

wherein the variable components are as herein described.

In a third embodiment, Ring A has the formula (A4):

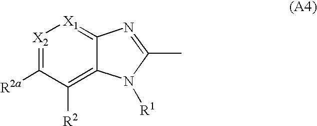

wherein the variable components are as herein described.

One particular class of rings of ring A are shown below, as formula A5:

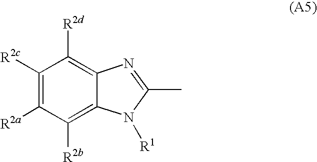

where each R^{2a}, R^{2b}, R^{2c} and R^{2d} are each independently selected from hydrogen, halogen, hydroxy, C₁₋₆alkyl, C₁₋₆halogenalkyl, C₃₋₁₂cycloalkyl, amino, C₁₋₆alkoxy, C₃₋₁₂cycloalkyloxy, sulphonate, sulphate, phosphate, quartenary ammonium, phosphonate, guanidimium or cyano; and the remaining variable components are as herein described.

In one embodiment, R^{2a}, R^{2c} and R^{2d} are all hydrogen; R^{2b} is selected from halogen, hydroxy, C₁₋₆alkyl, C₁₋₆halogenalkyl, C₃₋₁₂cycloalkyl, amino, C₁₋₆alkoxy, C₃₋₁₂cycloalkyloxy, sulphonate, sulphate, phosphate, quartenary ammonium, phosphonate, guanidimium or cyano; and the remaining variable components are as herein described; in said embodiment, R^{2b} is preferably selected from halogen and C₁₋₆alkyl.

In one embodiment, each R^{2a}, R^{2b}, R^{2c} and R^{2d} are each independently selected from trifluoromethyl, methoxy, hydrogen, methyl, fluoro and chloro; in another embodiment, each R^{2a}, R^{2b}, R^{2c} and R^{2d} are each independently selected from methoxy, hydrogen, methyl, fluoro and chloro. Each R^{2a} and R^{2b} are preferably hydrogen, fluoro or chloro, in particular chloro. Preferably, at least one of R^{2a} or R^{2b} is hydrogen. Preferably at least two of R^{2a}, R^{2b}, R^{2c} and R^{2d} are hydrogen.

R¹ may be selected from cyclopropyl, isopropyl, n-hexyl, n-pentyl, methyl, ethyl, methyl-cyclopropyl, iso-butyl, n-butyl and n-propyl. In particular, R¹ is selected from methyl-cyclopropyl, iso-butyl, n-butyl and n-propyl.

In one embodiment, R¹ is C₁₋₄alkyl. In one embodiment, R¹ is ethyl.

In preferred compounds, B is B1, wherein Y₄ represents N or CH.

In one embodiment, B is B1 and at least one of Y₁ and Y₂ is N.

Further preferably, Y₃ is CR³. Preferably at least one of Y₁, Y₂ and Y₄ is CR³.

R³ preferably represents hydrogen, halogen, C₁₋₆alkyl, C₃₋₁₂cycloalkyl, C₁₋₆alkoxy, C₃₋₁₂cycloalkoxy, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino. R³ more preferably represents hydrogen, fluoro, chloro or C₁₋₄ alkyl, e.g. methyl. R³ particularly preferably represents hydrogen or chloro.

In a further embodiment of the present invention, Ring B is of formula (B5):

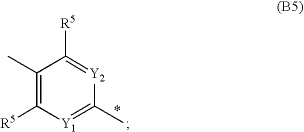

wherein the bond marked with the asterisk is attached to the group —NH—C; R⁵ is selected from hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino or cyano; and Y₁ and Y₂ are as herein described. Y₁ and Y₂ preferably represent N or CR³, wherein R³ preferably represents hydrogen or halogen, particular preferably hydrogen or chloro.

In further embodiments, Ring B is of formulae B6, B7 and B8:

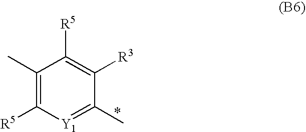

wherein the bond marked with the asterisk is attached to the group —NH—C; and R³, R⁵ and Y₁ are as herein described.

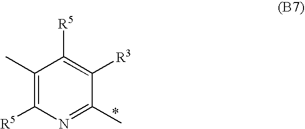

wherein the bond marked with the asterisk is attached to the group —NH—C; and R³ and R⁵ are as herein described.

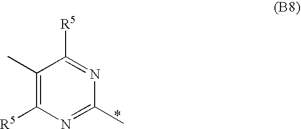

wherein the bond marked with the asterisk is attached to the group —NH—C; and R⁵ is as herein described.

For the formulae B5, B6, B7, B8, each R⁵ is preferably hydrogen.

In preferred compounds, C is C1, wherein Z₄ is CH and at least two of Z₁, Z₂ and Z₃ are N. In one embodiment of the present invention, Z₁ is CR³.

In a further embodiment, ring C has the formula (C5):

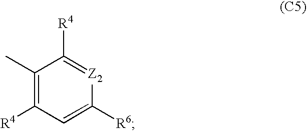

where R⁴ and Z₂ are as herein described; and R⁶ represents hydrogen, hydroxy, halogen, C₁₋₆alkyl or C₁₋₆alkoxy. R⁴ and R⁶ preferably represent hydroxy, halogen, C₁₋₆alkyl, C₃₋₁₂cycloalkyl, C₁₋₆alkoxy or C₃₋₁₂cycloalkoxy. R⁴ and R⁶ particularly preferably represent C₁₋₆alkyl, e.g. methyl.

In a further embodiment of the present invention, ring C has the formula (C6):

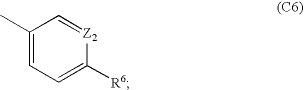

where R⁶ is selected from hydrogen, hydroxyl, C₁₋₆alkyl, C₃₋₁₂cycloalkyl, C₁₋₆alkoxy, C₃₋₁₂cycloalkoxy or halogen; and Z₂ is as herein described. R⁶ is preferably methyl, methoxy or halogen. R⁶ is further preferably chloro or fluoro.

In another embodiment, ring C is of the formula C7:

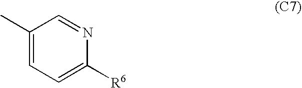

where R⁶ is as herein described.

In a further embodiment, ring C is of the formula C8:

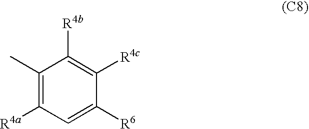

where R^{4a}, R^{4b} and R^{4c} are each independently selected from hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino or cyano; and R⁶ are as herein described. Preferably, R^{4a}, R^{4b} and R^{4c} are hydrogen. R⁶ is preferably selected from halogen, C₁₋₆alkyl, C₁₋₆alkoxy; R⁶ is further preferably chloro, methoxy or methyl.

In one embodiment of the present invention, the compounds have the formula (III):

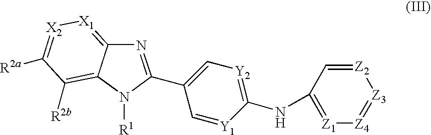

wherein

X₁, X₂ each independently represent CR² or N;

R^{2a}, R^{2b} each independently represent a group chosen from hydrogen, halogen, hydroxy, C₁₋₆alkyl, C₁₋₆halogenalkyl, C₃₋₁₂cycloalkyl, amino, C₁₋₆alkoxy, C₃₋₁₂cycloalkyloxy, sulphonate, sulphate, phosphate, quartenary ammonium, phosphonate and guanidimium;

R¹ is C₁₋₆alkyl or C₃₋₁₂cycloalkyl;

Y₁ and Y₂ each independently represent CR³ or N;

R³ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino or cyano;

Z₁, Z₂, Z₃, Z each independently represent CR⁴ or N, provided that at least one is CR⁴; and

R⁴ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino or di(C₃₋₁₂cycloalkyl)amino, cyano, C₁₋₆hydroxyalkyl, C₁₋₆alkoxycarbonyl or C₁₋₆alkylcarbonylamino.

In the embodiment, wherein the compounds have the formula (IV), preferably X₁, X₂ each independently represent CR² or N;

R^{2a}, R^{2b} each independently represent a group chosen from hydrogen, halogen, hydroxy, C₁₋₆alkyl, C₃₋₁₂cycloalkyl, amino, C₁₋₆alkoxy, C₃₋₁₂cycloalkyloxy, sulphonate, sulphate, phosphate, quartenary ammonium, phosphonate and guanidimium;

R¹ is C₁₋₆alkyl or C₃₋₁₂cycloalkyl;

Y₁ and Y₂ each independently represent CR³ or N;

R³ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino or cyano;

Z₁, Z₂, Z₃, Z each independently represent CR⁴ or N, provided that at least one is CR⁴; and

R⁴ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino or di(C₃₋₁₂cycloalkyl)amino.

In the embodiment, wherein the compounds have the formula (IV), further preferably X₁, X₂ each independently represent CR² or N;

R^{2a}, R^{2b} each independently represent a group chosen from hydrogen, halogen and C₁₋₆alkyl;

R¹ is C₁₋₆alkyl;

Y₁ and Y₂ each independently represent CR³ or N;

R³ represents hydrogen, halogen or C₁₋₆alkyl;

Z₁, Z₂, Z₃, Z₄ each independently represent CR⁴ or N, provided that at least one is CR⁴; and

R⁴ represents hydrogen, halogen or hydroxy, C₁₋₆alkyl

In a further embodiment, the compounds have the formula (IV):

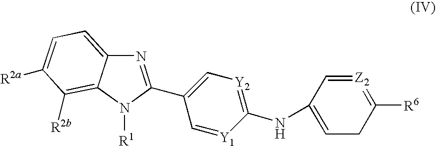

wherein

R^{2a}, R^{2b} each independently represent a group chosen from hydrogen, halogen, hydroxy, C₁₋₆alkyl, C₁₋₆halogenalkyl, C₃₋₁₂cycloalkyl, amino, C₁₋₆alkoxy, C₃₋₁₂cycloalkyloxy, sulphonate, sulphate, phosphate, quartenary ammonium, phosphonate and guanidimiumcyano;

R¹ is C₁₋₆alkyl or C₃₋₁₂cycloalkyl;

Y₁ and Y₂ each independently represent CR³ or N;

R³ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino or cyano;

Z₂ represents CR⁴ or N; and

R⁴ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino or di(C₃₋₁₂cycloalkyl)amino; and

R⁶ is selected from hydrogen, hydroxy, halogen, C₁₋₆alkyl, C₃₋₁₂cycloalkyl, C₁₋₆alkoxy, C₃₋₁₂cycloalkyloxy, cyano, C₁₋₆hydroxyalkyl, C₁₋₆alkoxycarbonyl and C₁₋₆alkylcarbonylamino.

In the embodiment, wherein the compounds have the formula (IV), preferably R^{2a}, R^{2b} each independently represent a group chosen from hydrogen, halogen, hydroxy, C₁₋₆alkyl, C₃₋₁₂cycloalkyl, amino, C₁₋₆alkoxy, C₃₋₁₂cycloalkyloxy, sulphonate, sulphate, phosphate, quartenary ammonium, phosphonate and guanidimium;

R¹ is C₁₋₆alkyl or C₃₋₁₂cycloalkyl;

Y₁ and Y₂ each independently represent CR³ or N;

R³ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino or cyano;

Z₂ represents CR⁴ or N; and

R⁴ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino or di(C₃₋₁₂cycloalkyl)amino; and

R⁶ is selected from hydrogen, hydroxy, halogen, C₁₋₆alkyl, C₃₋₁₂cycloalkyl, C₁₋₆alkoxy and C₃₋₁₂cycloalkyloxy.

In the embodiment, wherein the compounds have the formula (IV), further preferably R^{2a}, R^{2b} each independently represent a group chosen from hydrogen, halogen and C₁₋₆alkyl; further preferably R^{2a} is hydrogen;

R¹ is C₁₋₆alkyl;

Y₁ and Y₂ each independently represent CR³ or N;

R³ represents hydrogen, halogen or C₁₋₆alkyl;

Z₂ represents CR⁴ or N; and

R⁴ represents hydrogen, halogen or C₁₋₆alkyl; and

R⁶ is selected from hydrogen, halogen and C₁₋₆alkyl.

Specific examples of ring A are shown in groups Aa to Al below:

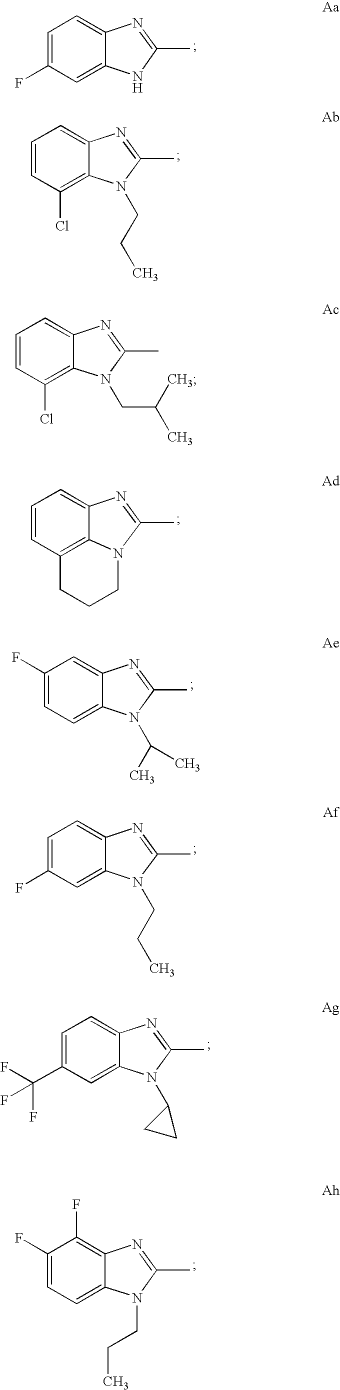

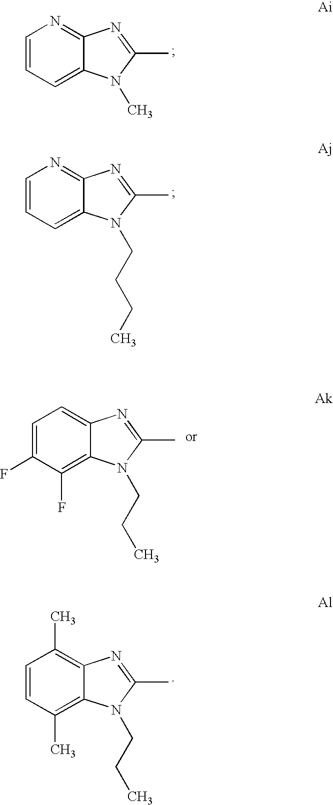

In all examples ring A is connected to ring B via the covalent bond shown at the right side of ring A.

Specific examples of ring B are shown in groups Ba to Bg below:

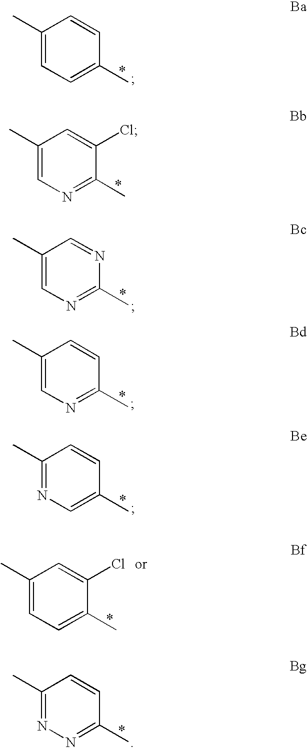

In all examples ring B is connected to ring A via the covalent bond shown at the left side of ring B; and to the group —NH—C via the covalent bond shown at the right side, which is further marked by an asterisk

Specific Examples of Ring C are shown below in groups Ca to Ck:

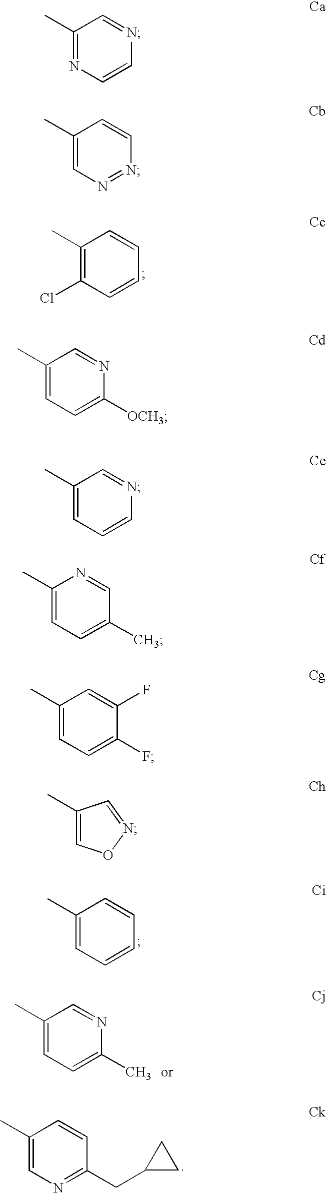

In all examples, ring C is connected to the nitrogen atom of the amine group via the covalent bond shown at the left side of ring C.

The above mentioned general or preferred radical definitions apply both to the end products and also, correspondingly, to the starting materials or intermediates required in each case for the preparation. These radical definitions can be combined with one another at will, i.e. including combinations between the given preferred ranges. Further, individual definitions may not apply.

It will be appreciated that each of the different components of the compounds of the present invention may be combined in many different ways. As an example, each of the formulae A, A1, A2, A3, A4 or A5 may be combined with any of the formulae B, B1, B2, B3, B4, B5, B6, B7 or B8. The resulting product of such a combined moiety may then be combined with any of the moieties C, C1, C2, C3, C4, C5, C6, C7 or C8. As a result, the following combinations are possible, as an example:

| Ring | Ring | Ring |
|---|---|---|
| A | B | C |
| A | B | C |
| A | B | C1 |
| A | B | C5 |
| A | B | C6 |
| A | B | C7 |
| A | B | C8 |
| A | B1 | C |
| A | B1 | C1 |
| A | B1 | C5 |
| A | B1 | C6 |
| A | B1 | C7 |
| A | B1 | C8 |
| A | B5 | C |
| A | B5 | C1 |
| A | B5 | C5 |
| A | B5 | C6 |
| A | B5 | C7 |
| A | B5 | C8 |
| A | B6 | C |
| A | B6 | C1 |
| A | B6 | C5 |
| A | B6 | C6 |
| A | B6 | C7 |
| A | B6 | C8 |
| A | B7 | C |
| A | B7 | C1 |
| A | B7 | C5 |
| A | B7 | C6 |
| A | B7 | C7 |
| A | B7 | C8 |
| A1 | B | C |
| A1 | B | C1 |
| A1 | B | C5 |
| A1 | B | C6 |
| A1 | B | C7 |
| A1 | B | C8 |
| A1 | B1 | C |
| A1 | B1 | C1 |
| A1 | B1 | C5 |
| A1 | B1 | C6 |
| A1 | B1 | C7 |
| A1 | B1 | C8 |
| A1 | B5 | C |
| A1 | B5 | C1 |
| A1 | B5 | C5 |
| A1 | B5 | C6 |
| A1 | B5 | C7 |
| A1 | B5 | C8 |
| A1 | B6 | C |
| A1 | B6 | C1 |
| A1 | B6 | C5 |
| A1 | B6 | C6 |
| A1 | B6 | C4 |
| A1 | B6 | C5 |
| A1 | B7 | C |
| A1 | B7 | C1 |
| A1 | B7 | C5 |
| A1 | B7 | C6 |
| A1 | B7 | C7 |
| A1 | B7 | C8 |
| A2 | B | C |
| A2 | B | C1 |
| A2 | B | C5 |
| A2 | B | C6 |
| A2 | B | C7 |
| A2 | B | C8 |
| A2 | B1 | C |
| A2 | B1 | C1 |
| A2 | B1 | C5 |
| A2 | B1 | C6 |
| A2 | B1 | C7 |
| A2 | B1 | C8 |
| A2 | B5 | C |
| A2 | B5 | C1 |
| A2 | B5 | C5 |
| A2 | B5 | C6 |
| A2 | B5 | C7 |
| A2 | B5 | C8 |
| A2 | B6 | C |
| A2 | B6 | C1 |
| A2 | B6 | C5 |
| A2 | B6 | C6 |
| A2 | B6 | C7 |
| A2 | B6 | C8 |
| A2 | B7 | C |
| A2 | B7 | C1 |
| A2 | B7 | C5 |
| A2 | B7 | C6 |
| A2 | B7 | C7 |
| A2 | B7 | C8 |
| A3 | B | C |
| A3 | B | C1 |
| A3 | B | C5 |
| A3 | B | C6 |
| A3 | B | C7 |
| A3 | B | C8 |
| A3 | B1 | C |
| A3 | B1 | C1 |
| A3 | B1 | C5 |
| A3 | B1 | C6 |
| A3 | B1 | C7 |
| A3 | B1 | C8 |
| A3 | B5 | C |
| A3 | B5 | C1 |
| A3 | B5 | C5 |
| A3 | B5 | C6 |
| A3 | B5 | C7 |
| A3 | B5 | C8 |
| A3 | B6 | C |
| A3 | B6 | C1 |
| A3 | B6 | C5 |
| A3 | B6 | C6 |
| A3 | B6 | C7 |
| A3 | B6 | C8 |
| A3 | B7 | C |
| A3 | B7 | C1 |
| A3 | B7 | C5 |
| A3 | B7 | C6 |
| A3 | B7 | C7 |
| A3 | B7 | C8 |
| A4 | B | C |
| A4 | B | C1 |
| A4 | B | C5 |
| A4 | B | C6 |
| A4 | B | C7 |
| A4 | B | C8 |
| A4 | B1 | C |
| A4 | B1 | C1 |
| A4 | B1 | C5 |
| A4 | B1 | C6 |
| A4 | B1 | C7 |
| A4 | B1 | C8 |
| A4 | B5 | C |
| A4 | B5 | C1 |
| A4 | B5 | C5 |
| A4 | B5 | C6 |
| A4 | B5 | C7 |
| A4 | B5 | C8 |
| A4 | B6 | C |
| A4 | B6 | C1 |
| A4 | B6 | C5 |
| A4 | B6 | C6 |
| A4 | B6 | C7 |
| A4 | B6 | C8 |
| A4 | B7 | C |
| A4 | B7 | C1 |
| A4 | B7 | C5 |
| A4 | B7 | C6 |
| A4 | B7 | C7 |
| A4 | B7 | C8 |
| A5 | B | C |
| A5 | B | C1 |
| A5 | B | C5 |
| A5 | B | C6 |
| A5 | B | C7 |
| A5 | B | C8 |
| A5 | B1 | C |
| A5 | B1 | C1 |
| A5 | B1 | C5 |
| A5 | B1 | C6 |
| A5 | B1 | C7 |
| A5 | B1 | C8 |
| A5 | B5 | C |
| A5 | B5 | C1 |
| A5 | B5 | C5 |
| A5 | B5 | C6 |
| A5 | B5 | C7 |
| A5 | B5 | C8 |
| A5 | B6 | c |
| A5 | B6 | C1 |
| A5 | B6 | C5 |
| A5 | B6 | C6 |
| A5 | B6 | C7 |
| A5 | B6 | C8 |
| A5 | B7 | C |
| A5 | B7 | C1 |
| A5 | B7 | C5 |
| A5 | B7 | C6 |
| A5 | B7 | C7 |
| A5 | B7 | C8 |

The compounds of the present invention may be assembled by the building blocks of individual rings A, B and C. As such, the compounds of the present invention lend themselves very well to synthetic routes involving library synthesis and the like.

As an example, a ring A may first be synthesized by conventional techniques, for example as disclosed in the Examples, and then connected to a ring B. The connection of ring A to ring B may comprise known synthetic techniques, such as condensation reaction and/or carbon-carbon bond forming techniques. The resulting ring A-ring B compound may then be further connected to a ring C compound by known chemical techniques.

As will be appreciated, the combining of compounds need not be conducted in the order ring A to ring B to ring C. All combinations are contemplated. In addition the combining of moieties that are not finalized ring structures, i.e. ring structures that require further modifications, e.g. a pre-Ring A moiety to a pre-ring B moiety are also contemplated. For example, upon combination with ring B, ring A may then be further modified prior to the coupling reaction of the resulting compound to ring C.

To this end, the present invention also provides intermediate templates of two or more ring components A, B and/or C, which may be further modified by known chemical techniques to produce libraries and/or families of compounds having a similar backbone structure. Said intermediates are part of the invention.

Example syntheses for forming compounds of the present invention are shown below and are exemplified in the Examples:

### EXAMPLE GENERAL SYNTHESIS 1

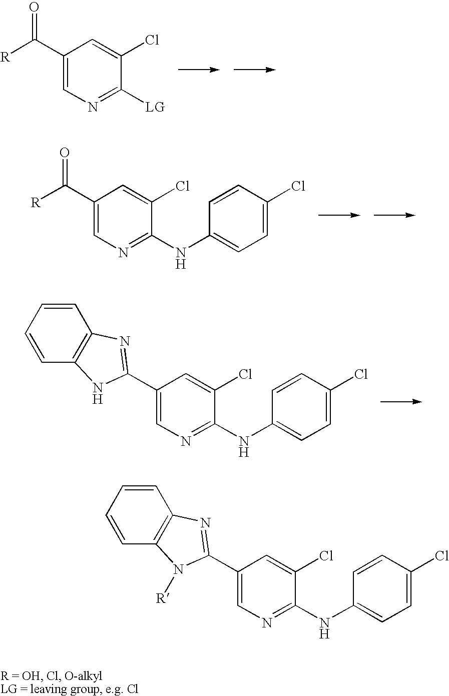

### EXAMPLE GENERAL SYNTHESIS 2

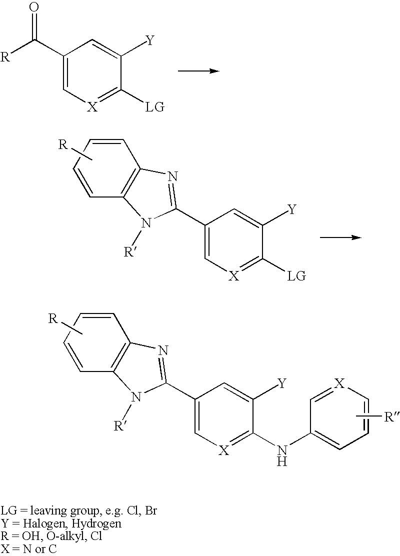

### EXAMPLE GENERAL SYNTHESIS 3

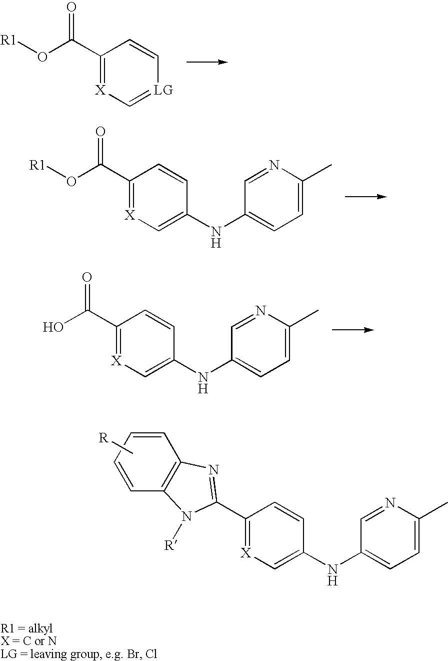

### EXAMPLE GENERAL SYNTHESIS 4

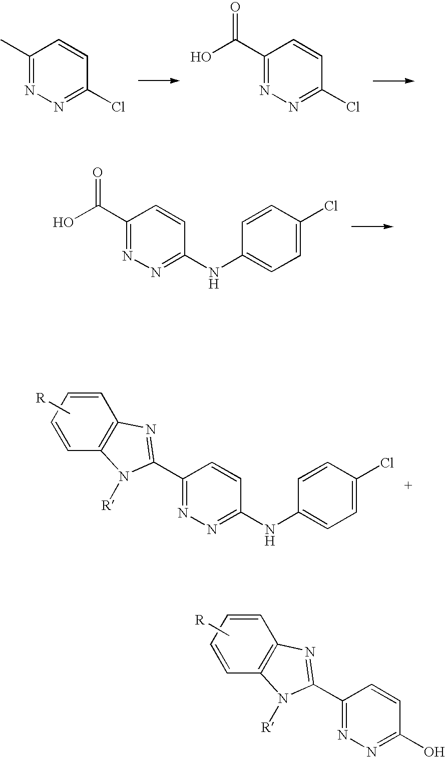

### EXAMPLE GENERAL SYNTHESIS 5

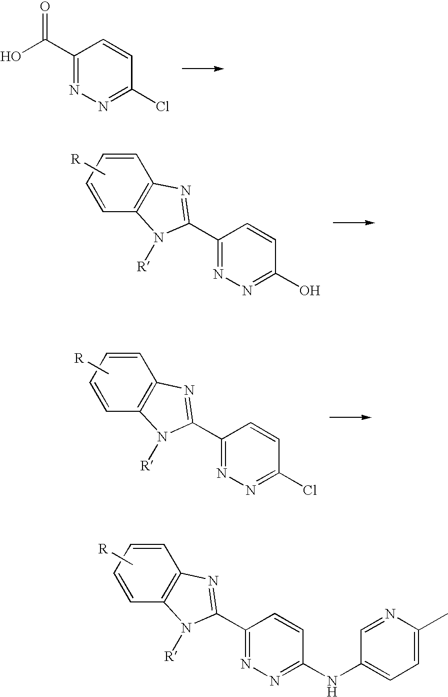

### EXAMPLE GENERAL SYNTHESIS 6

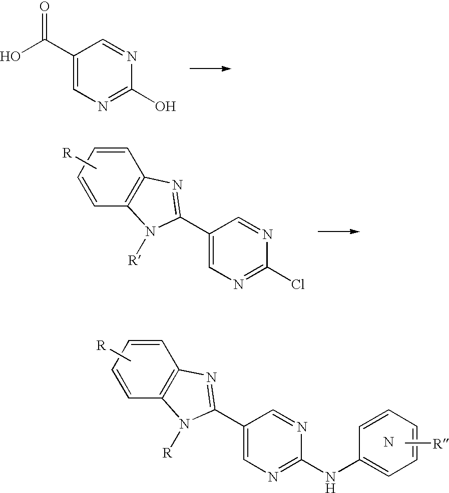

### EXAMPLE GENERAL SYNTHESIS 7

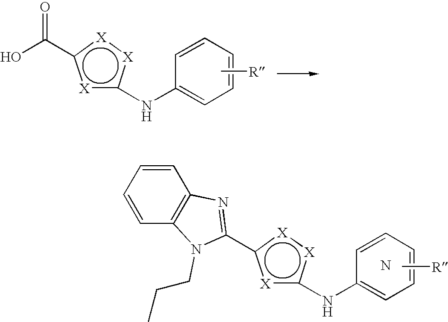

The following considerations apply to the individual reaction steps described above:

a) One or more functional groups, for example carboxy, hydroxy, amino, or mercapto, may need to be protected in the starting materials by protecting groups. The protecting groups employed may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter. The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosäuren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide und Derivate” (Chemistry of carbohydrates: monosaccharides and derivatives), Georg Thieme Verlag, Stuttgart 1974.
b) Acid addition salts may be produced from the free bases in known manner, and vice-versa. Compounds of formulae (I), (II), (III) and (IV) in optically pure form can be obtained from the corresponding racemates according to well-known procedures, e.g. HPLC with chiral matrix. Alternatively, optically pure starting materials can be used.
c) Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula (I) itself. Enantiomers may be separated through the formation of diastereomeric salts, for example by salt formation with an enantiomer-pure chiral acid, or by means of chromatography, for example by HPLC, using chromatographic substrates with chiral ligands.
d) Suitable diluents for carrying out the above—described are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile propionitrile or butyronitrile; amides, such as N,N-dimethylformamide, N,N-dimethylacetamide, N-methyl-formanilide, N-methyl-pyrrolidone or hexamethylphosphoric triamide; esters, such as methyl acetate or ethyl acetate, sulphoxides, such as dimethyl sulphoxide, alcohols, such as methanol, ethanol, n- or i-propanol, ethylene glycol monomethyl ether, ethylene glycol monoethyl ether, diethylene glycol monomethyl ether, diethylene glycol monoethyl ether. Further, mixtures of diluents may be employed. Depending on the starting materials, reaction conditions and auxiliaries, water or diluents containing water may be suitable. It is also possible to use one a starting material as diluent simultaneously.
e) Reaction temperatures can be varied within a relatively wide range. In general, the processes are carried out at temperatures between 0° C. and 150° C., preferably between 10° C. and 120° C. Deprotonation reactions can be varied within a relatively wide range. In general, the processes are carried out at temperatures between −150° C. and +50° C., preferably between −75° C. and 0° C.
f) The reactions are generally carried out under atmospheric pressure. However, it is also possible to carry out the processes according to the invention under elevated or reduced pressure—in general between 0.1 bar and 10 bar.
g) Starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred at the required temperature for a number of hours.
h) Work-up is carried out by customary methods (cf. the Preparation Examples).
i) A compound of formulae (I), (II), (III) and (IV) obtained according to the above described processes can be converted into another compound formulae (I), (II), (III) and (IV) according to conventional methods.

Compounds of formulae (I), (II), (III) and (IV) and their pharmaceutically acceptable acid addition salts, hereinafter referred to as agents of the invention, exhibit valuable pharmacological properties and are therefore useful as pharmaceuticals.

In particular, the agents of the invention exhibit a marked and selective modulating, especially antagonistic, action at human metabotropic glutamate receptors (mGluRs). This can be determined in vitro for example at recombinant human metabotropic glutamate receptors, especially PLC-coupled subtypes thereof such as mGluR5, using different procedures like, for example, measurement of the inhibition of the agonist induced elevation of intracellular Ca²⁺ concentration in accordance with L. P. Daggett et al., Neuropharm. Vol. 34, pages 871-886 (1995), P. J. Flor et al., J. Neurochem. Vol. 67, pages 58-63 (1996) or by determination to what extent the agonist induced elevation of the inositol phosphate turnover is inhibited as described by T. Knoepfel et al., Eur. J. Pharmacol. Vol. 288, pages 389-392 (1994), L. P. Daggett et al., Neuropharm. Vol. 67, pages 58-63 (1996) and references cited therein. Isolation and expression of human mGluR subtypes are described in U.S. Pat. No. 5,521,297. Selected agents of the invention show IC50 values for the inhibition of the agonist (e.g. glutamate or quisqualate) induced elevation of intracellular Ca2+ concentration or the agonist (e.g. glutamate or quisqualate) induced inositol phosphate turnover, measured in recombinant cells expressing hmGluR^{5a} of about 0.1 nM to about 50 μM.

The agents of the invention are therefore useful in the prevention, treatment or delay of progression of disorders associated with irregularities of the glutamatergic signal transmission, of the gastro-intestinal and urinary tract and of nervous system disorders mediated full or in part by mGluR5.

Disorders associated with irregularities of the glutamatergic signal transmission are for example epileptogenesis including neuronal protection after status epilepticus, cerebral ischemias, especially acute ischemias, ischemic diseases of the eye, muscle spasms such as local or general spasticity, skin disorders, obesity disorders and, in particular, convulsions or pain.

Disorders of the gastrointestinal tract include Gastro-Esophageal Reflux Disease (GERD), Functional Gastro-intestinal Disorders and Post-operative Ileus.

Functional Gastro-intestinal Disorders (FGIDs) are defined as chronic or recurrent conditions associated with abdominal symptoms without organic cause using conventional diagnostic measures. A cardinal symptom present in many FGIDs is visceral pain and/or discomfort. FGIDs include functional dyspepsia (FD), functional heartburn (a subset of GERD), irritable bowel syndrome (IBS), functional bloating, functional diarrhea, chronic constipation, functional disturbancies of the biliary tract as well as other conditions according to Gut 1999; Vol. 45 Suppl. II. A disorder of particular interest is GERD.

Post-operative Ileus is defined as failure of aboral passage of intestinal contents due to transient impairment of GI motility following abdominal surgery.

Disorders of the Urinary Tract comprise conditions associated with functional disturbancies and/or discomfort/pain of the urinary tract. Examples of disorders of the urinary tract include but are not limited to incontinence, benign prostatic hyperplasia, prostatitis, detrusor hyperreflexia, outlet obstruction, urinary frequency, nocturia, urinary urgency, overactive bladder (OAB), pelvic hypersensitivity, urge incontinence, urethritis, prostatodynia, cystitis, idiopathic bladder hypersensitivity and the like. OAB is a syndrome characterized by urgency, with or without urinary incontinence, and usually with increased voiding frequency and nocturia.

Nervous system disorders mediated full or in part by mGluR5 are for example acute, traumatic and chronic degenerative processes of the nervous system, such as Parkinson's disease, Parkinson's dyskinesia (e.g. L-dopa induced dyskinesia), dyskinesias induced by neuroleptics (e.g. tardive dyskenisia), Tic disorders, Tourette Syndrome, Restless Leg Syndrome, Periodic Limb Movement Syndromes, senile dementia, Alzheimer's disease, Huntington's chorea, amyotrophic lateral sclerosis, multiple sclerosis and fragile X syndrome, substance-related disorders, psychiatric diseases such as schizophrenia, affective and anxiety disorders, attention deficit disorders and cognitive dysfunction associated with these and other CNS disorders. Substance-related disorders include substance abuse, substance dependence and substance withdrawal disorders, e.g. nicotine withdrawal. Anxiety disorders includes panic disorder, social and specific phobias, anxiety, obsessive compulsive disorder (OCD), post traumatic stress disorder (PTSD) and generalized anxiety disorder (GAD). Affective disorders include depressive (major depression, dysthymia, depressive disorders NOS) and bipolar disorders (bipolar I and II disorders). Cognitive dysfunction associated with these and other CNS disorders include deficits and abnormalities in attention and vigilance, executive functions and memory (for instance working memory and episodic memory). Other disorders which are mediated fully or in part by mGluR5 are pain and itch. A disorder of particular interest is Parkinson's dyskinesia induced by L-dopa.

The agents of the present invention may also be useful for treating or preventing migraine.

The agents of the present invention may also be useful for inflammatory diseases, such as pain, inflammation and/or oedema consequential to trauma, for example associated with burns, sprains, fractures or the like, inflammatory airways diseases, such as COPD, asthma, rhinitis, inflammatory bowel disease, cystitis, uveitis, inflammatory skin disorders, such as psoriasis or eczema, rheumatoid arthritis, use as a smooth muscle relaxant, for example for the treatment of spasms of the gastro-intestinal tract or uterus, for example in the therapy of Crohn's disease, ulcerative collitis or pancreatitis, or for the treatment of muscle spasticity and tremor, for example in multiple sclerosis, teno-synovitis, gout, ocular disorders, for example glaucoma, cough.

The agents of the present invention may also be useful for treating cognitive impairment and/or attention deficit disorder.

Cognitive dysfunction include deficits and abnormalities in attention and vigilance, executive functions and memory (for instance working memory and episodic memory). Other disorders relating to cognitive dysfunction include sleep related breathing disorders (SRBD), behavioral impairments, information processing deficits and age-related disorders. Further examples falling of cognitive impairment and/or attention deficit disorders include: Attention-deficit hyperactivity disorder (ADHD), childhood ADHD, adult ADHD, excess daytime somnolence, sleep apnea, shift-worker's sleep-wake cycle disruption, traumatic brain injury, neurodegenerative disorders with associated memory and cognitive problems (such as Alzheimer's disease, Lewy body dementia, senile dementia, vascular dementia, Parkinson's disease), chronic fatigue syndrome, fatigue associated with sleep deprivation or prolonged wakefulness, age-related decline in memory and cognitive function (such as mild cognitive impairment), cognitive impairment associated with mood disorders (such as depression) and anxiety, schizophrenia, day time sleepiness associated with narcolepsy.

Furthermore, the agents of the present invention may provide treatment for or improve of the cognitive enhancement of a subject. The term “cognitive enhancement” includes, but is not limited to, cognition enhancement, vigilance, counteracting effects of fatigue, enhancing alertness, attention, memory (working, episodic), learning ability, reaction time, cognitive performance enhancement, excess daytime somnolence, reversal of information processing deficits, improvement of disorganization, i.e. improving organizational skills/level of organizational ability.

The agents of the present invention may also be useful for treating pervasive developmental disorders (PDD). PDD is a group of diseases characterized by a delay in the development of socialization and communications skills. The following diseases are part of the PDD: Autism, Asperger's syndrome, childhood disintegrative disorder, and Rett's syndrome, and fragile X. The main symptomatology are: Autistic-like behavior, repetitive behavior (OCD), in some cases irritability, and ADHS. Fragile X Syndrome have two different genotype-phenotype: Full mutation (mental retardation, ADHD, autism, and anxiety), partial mutation (tremor-ataxia, parkinsonism, anxiety). A disorder of particular interest is fragile X syndrome.

The agents of the present invention may be useful for the prevention of the above-mentioned conditions and disorders.

The agents of the present invention may be useful for the treatment of the above-mentioned conditions and disorders.

The agents of the present invention may be useful for the delay of progression of the above-mentioned conditions and disorders.

The usefulness of the agents of the invention in the treatment of the above-mentioned disorders can be confirmed in a range of standard tests including those indicated below:

Activity of the agents of the invention in anxiety can be demonstrated in standard models such as the stress-induced hyperthermia in mice [cf. A. Lecci et al., Psychopharmacol. 101, 255-261]. At doses of about 0.1 to about 30 mg/kg p.o., selected agents of the invention reverse the stress-induced hyperthermia.

At doses of about 4 to about 50 mg/kg p.o., selected agents of the invention show reversal of Freund complete adjuvant (FCA) induced hyperalgesia [cf. J. Donnerer et al., Neuroscience 49, 693-698 (1992) and C. J. Woolf, Neuroscience 62, 327-331 (1994)].

Activity of the agents of the invention in GERD can be demonstrated in standard models such as the gastric distension-induced transient lower esophageal sphincter relaxations (TLESRs) in dogs. At doses of about 0.03 to about 10 mg/kg p.o., selected agents of the invention reduce the occurrence of TLESRs.

Activity of the agents of the invention in functional dyspepsia can be demonstrated a model of fasted gastric tone and gastric accommodation to meal in dogs. At doses of about 0.03 to about 10 mg/kg p.o., selected agents of the invention increase the gastric volume in fasting conditions indicative of a reduced gastric tone.

Activity of the agents of the invention in visceral hyperalgesia can be demonstrated in standard rat models according to modified methods by Tarrerias, A. et al., Pain (2002) 100: 91-97, Schwetz, l. et al., Am. J. Physiol. (2005) 286: G683-G691, of La, J. et al., World J. Gastroenterol. (2003) 9: 2791-2795. At doses of about 0.03 to about 30 mg/kg p.o., selected agents of the invention reduce the exaggerated abdominal striated muscle contractions, indicative of a visceral antinociceptive activity.

Activity of the agents of the invention in visceral sensation/pain of the urinary bladder can be demonstrated in a standard mouse model according to a modified method by Ness T J and Elhefni H. J Urol. (2004) 171:1704-8. At doses of about 0.3 to about 30 mg/kg p.o., selected agents of the invention reduce the EMG (visceromotor) response, indicative of a visceral antinociceptive and/or hyposensitivity.

Activity of the agents of the invention in overactive bladder and urge incontinence can be demonstrated in standard cystometry models in rats according to modified method by Tagaki-Matzumoto et al J. Pharmacol. Sci. (2004) 95: 458-465. At doses of about 0.03 to about 10 mg/kg p.o., selected agents of the invention increased threshold volumes eliciting bladder contractions indicative of therapeutic potential in conditions with bladder dysfunctions.

For all the above mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.05 to about 100 mg/kg animal body weight. In larger mammals, for example humans, an indicated daily dosage is in the range from about 5 to 1500 mg, preferably about 10 to about 1000 mg of the compound conveniently administered in divided doses up to 4 times a day or in sustained release form.

In accordance with the foregoing, the present invention also provides in a further aspect an agent of the invention for use as a medicament, e.g. in the treatment of disorders associated with irregularities of the glutamatergic signal transmission, and of nervous system disorders mediated full or in part by mGluR5.

The invention also provides the use of an agent of the invention, in the treatment of disorders associated with irregularities of the glutamatergic signal transmission, and of nervous system disorders mediated full or in part by mGluR5.

In a further aspect, the invention provides the use of compounds of formulae (I), (II), (III), (IV) and (V) as modulators of metabotropic Glutamate Receptors, Subtype 5 (“mGluR5-Modulators”).

Furthermore the invention provides the use of an agent of the invention for the manufacture of a pharmaceutical composition designed for the treatment of disorders associated with irregularities of the glutamatergic signal transmission, and of nervous system disorders mediated full or in part by mGluR5.

Furthermore the invention provides an agent of the invention for the prevention, treatment or delay of progression of: disorders associated with irregularities of the glutamatergic signal transmission, the gastro-intestinal and urinary tract and nervous system disorders mediated full or in part by mGluR5.

In a further aspect the invention relates to a method of treating disorders mediated full or in part by mGluR5, which method comprises administering to a warm-blooded organism in need of such treatment a therapeutically effective amount of an agent of the invention.

Moreover the invention relates to a pharmaceutical composition comprising an agent of the invention in association with one or more pharmaceutical carrier or one or more pharmaceutically acceptable diluent.

The pharmaceutical compositions according to the invention are compositions for enteral, such as nasal, rectal or oral, or parenteral, such as intramuscular or intravenous, administration to warm-blooded animals (human beings and animals) that comprise an effective dose of the pharmacological active ingredient alone or together with a significant amount of a pharmaceutically acceptable carrier. The dose of the active ingredient depends on the species of warm-blooded animal, body weight, age and individual condition, individual pharmacokinetic data, the disease to be treated and the mode of administration.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Preferred are the compounds according to the examples.

Further, properly isotope-labeled agents of the invention exhibit valuable properties as histopathological labeling agents, imaging agents and/or biomarkers, hereinafter “markers”, for the selective labeling of the metabotropic glutamate receptor subtype 5 (mGlu5 receptor). More particularly the agents of the invention are useful as markers for labeling the central and peripheral mGlu5 receptors in vitro or in vivo. In particular, compounds of the invention which are properly isotopically labeled are useful as PET markers. Such PET markers are labeled with one or more atoms selected from the group consisting of ¹¹C, ¹³N, ¹⁵O, ¹⁸F.

The agents of the invention are therefore useful, for instance, for determining the levels of receptor occupancy of a drug acting at the mGlu5 receptor, or diagnostic purposes for diseases resulting from an imbalance or dysfunction of mGlu5 receptors, and for monitoring the effectiveness of pharmacotherapies of such diseases.

In accordance with the above, the present invention provides an agent of the invention for use as a marker for neuroimaging.

In a further aspect, the present invention provides a composition for labeling brain and peripheral nervous system structures involving mGlu5 receptors in vivo and in vitro comprising an agent of the invention.

In still a further aspect, the present invention provides a method for labeling brain and peripheral nervous system structures involving mGlu5 receptors in vitro or in vivo, which comprises contacting brain tissue with an agent of the invention.

The method of the invention may comprise a further step aimed at determining whether the agent of the invention labeled the target structure. Said further step may be effected by observing the target structure using positron emission tomography (PET) or single photon emission computed tomography (SPECT), or any device allowing detection of radioactive radiations.

The following non-limiting Examples illustrate the invention. A list of Abbreviations used is given below.
AcOH acetic acid
aq aqueous
BOC tert-butoxycarbonyl
n-BuLi n-butyl lithium
DMF N,N′-dimethylformamide
AcN acetonitrile
BINAP (2,2-bis(diphenylphosphino)-1,1-binaphthyl
DCE 1,2-dichloroethane
DCM dichloromethane
DIPEA N,N-diisopropylethylamine
DMA N,N-dimethylacetamide
DMAP 4-N,N-dimethylaminopyridine
DME 1,2-dimethoxyethane
DMSO dimethylsulfoxide
EtOAc ethylacetate
ESI electrospray ionization
h hours
hex hexanes
HCl hydrochloric acid
HPLC high pressure liquid chromatography
min minutes
Mp melting point
MS mass spectroscopy
MTBE methyl-tert.-butylether
NMP N-methyl-pyrrolidone
org organic
pH pH-value
PPA polyphosphoric acid
p-TsOH para-touluene sulfonic acid
R_{f} retention factor (Thin Layer Chromatography)
RT room temperature
t_{R} retention time
TFA trifluoroacetic acid
THF tetrahydrofuran
TLC thin layer chromatography
HPLC ultra performance liquid chromatography
HPLC Specificity

Waters Acquity system equipped with a sample manager and a PDA detector operating at a wavelength range between 220 and 400 nm. Column Acquity HPLC BEH C₁₈ (1.7 μm, 50×2.1 mm), temperature: 35° C., flow: 0.6 mL min⁻¹. Eluents: Water+0.1% TFA/Acetonitrile+0.1% TFA from 95/5 to 0/100 over 2 min.

### HPLC Specificity

System 1: Agilent 1100 Series, LC-MSD and a Agilent Zorbax SB-C18 3×30 mm 1.8 μm Column running a gradient Water+0.05% TFA/Acetonitrile+0.05% TFA from 100/0 to 0/100 over 3.25-0/100 over 0.75-0/100 to 90/10 over 0.25 with a flux of 0.7 ml/min, 35° C.

System 2: Agilent 1100 Series, LC-MSD and a Agilent Zorbax SB-C18 3×30 mm 1.8 μm Column running a gradient Water+0.05% TFA/Acetonitrile+0.05% TFA from 90/10 to 0/100 over 3.25-0/100 over 0.75-0/100 to 70/30 over 0.25 with a flux of 0.7 ml/min, 35° C.

System 3: Agilent 1100 Series, LC-MSD and a Agilent Zorbax SB-C18 3×30 mm 1.8 μm Column running a gradient Water+0.05% TFA/Acetonitrile+0.05% TFA from 70/30 to 0/100 over 3.25-0/100 over 0.75-0/100 to 60/40 over 0.25 with a flux of 0.7 ml/min, 35° C.

System 4: Agilent 1100 Series, LC-MSD and a Agilent Zorbax SB-C18 3×30 mm 1.8 μm Column running a gradient Water+0.05% TFA/Acetonitrile+0.05% TFA from 60/40 to 0/100 over 3.25-0/100 over 0.75-0/100 to 60/40 over 0.25 with a flux of 0.7 ml/min, 35° C.

System 5: Agilent 1100 Series, LC-MSD and a Agilent Zorbax SB-C18 3×30 mm 1.8 μm Column running a gradient Water+0.05% TFA/Acetonitrile+0.05% TFA from 30/70 to 0/100 over 3.25-0/100 over 0.75-0/100 to 90/10 over 0.25 with a flux of 0.7 ml/min, 35° C.

Unsubstituted and substituted N-alkyl-benzene-1,2-diamine building blocks can be prepared according to literature procedures, or as described below:

### N-Ethyl-benzene-1,2-diamine

[J. T. Ralph, Synth. Comm. (1989) 19, 7-8, 1381-1387]

### N-Propyl-benzene-1,2-diamine

[J. T. Ralph, Synth. Comm. (1989) 19, 7-8, 1381-1387]

### N-Butyl-benzene-1,2-diamine

[J. T. Ralph, Synth. Comm. (1989) 19, 7-8, 1381-1387]

### N-Pentyl-benzene-1,2-diamine

[J. T. Ralph, Synth. Comm. (1989) 19, 7-8, 1381-1387]

### N-Isobutyl-benzene-1,2-diamine

[Y.-M. Legrand, M. Gray, G. Cooke, V. M. Rotello, J. Am. Chem. Soc. (2003) 125, 51, 15789-15795]

### N-1-Propyl-4-trifluoromethyl-benzene-1,2-diamine

A solution of 1-chloro-2-nitro-4-trifluoromethyl-benzene (4 ml, 27.0 mmol) and propylamine (6.7 ml, 80.0 mmol) in EtOH (6 ml) and water (1 ml) was heated to 130° C. for 18 h in a sealed tube. The mixture was then diluted with water and extracted with EtOAc. The combined org. phases were dried over Na₂SO₄, filtered and concentrated in vacuo. Purification by flash chromatography (Hex/EtOAc 100:0 to 90:10) to gave (2-nitro-4-trifluoromethyl-phenyl)-propyl-amine (6.92 g), which was diluted in anhydrous THF (100 ml) and treated with Pd/C (10%, 500 mg). The mixture was stirred under H₂ at RT for 3 h, and then filtered and concentrated in vacuo to give the desired product (6.7 g, quantitative). HPLC (System 3, 30-100% CH₃CN): t_{R}=2.044 min, MS (ES+): 219 [M+1].

### 4-Methyl-N-1-propyl-benzene-1,2-diamine

[H. G\ddot{k}er, C. Kus, D. W. Boykin, S. Yildiz, N. Altanlar, Bioorg. Med. Chem. (2002), 10, 2589-2596]

### 4-Methyl-N-2-propyl-benzene-1,2-diamine

A solution of 2-chloro-4-methyl-1-nitro-benzene (3.54 g, 20.0 mmol) and propylamine (5.0 ml, 60.0 mmol) in EtOH (5 ml) and water (1 ml) was heated to 130° C. for 96 h in a sealed tube. The mixture was then diluted with water and extracted with EtOAc. The combined org. phases were dried over Na₂SO₄, filtered and concentrated in vacuo. Purification by flash chromatography (Hex/EtOAc 100:0 to 90:10) gave (5-methyl-2-nitro-phenyl)-propyl-amine (3.55 g), which was diluted in anhydrous THF (100 ml) and treated with Pd/C (10%, 500 mg). The mixture was stirred under H₂ at RT for 1 h, and then filtered and concentrated in vacuo. Purification by flash chromatography (10:0 to 80:20) provided the desired product (2.14 g, 72%). HPLC (System 2, 30-100% CH₃CN): t_{R}=0.414 min, MS (ES+): 165 [M+1].

### 3-Methyl-N-2-propyl-benzene-1,2-diamine

A solution of 2-chloro-1-methyl-3-nitro-benzene (3.54 ml, 26.2 mmol) and propylamine (6.57 ml, 78.8 mmol) in EtOH (5 ml) and water (1 ml) was heated to 140° C. for 192 h in a sealed tube. The mixture was then diluted with water and extracted with EtOAc. The combined org. phases were dried over Na₂SO₄, filtered and concentrated in vacuo. Filtration on silica (Hex/EtOAc 100:0 to 90:10) gave (2-methyl-6-nitro-phenyl)-propyl-amine (2.6 g, 13.4 mmol), which was diluted in anhydrous THF (100 ml) and treated with Pd/C (10%, 260 mg). The mixture was stirred under H₂ at RT for 1 h. The mixture was then filtered and concentrated in vacuo. Purification by flash chromatography (Hex/EtOAc 100:0 to 80:20) provided the desired product (2.03 g, 92%). HPLC (System 2, 10-100% CH₃CN): t_{R}=2.075 min, MS (ES+): 165 [M+1].

### 3-Methyl-N-1-propyl-benzene-1,2-diamine

A solution of (3-methyl-2-nitro-phenyl)-propyl-amine (3.8 g, 19.6 mmol) in THF (100 ml) was treated with Pd/C (10%, 250 mg) and the mixture was stirred under H₂ at RT for 15 h. The mixture was then filtered and concentrated in vacuo. Purification by flash chromatography (Hex/EtOAc 100:0 to 80:20) provided the desired product (493 mg, 15%). HPLC (System 2, 10-100% CH₃CN): t_{R}=2.255 min, MS (ES+): 165 [M+1].

The starting material can be prepared as described hereafter:

### (3-Methyl-2-nitro-phenyl)-propyl-amine

A solution of 1-bromo-3-methyl-2-nitro-benzene (4.45 g, 20.0 mmol) and propylamine (5.0 ml, 60.0 mmol) in EtOH (5 ml) and water (1 ml) was heated to 160° C. for 7.5 h in a microwave oven. The mixture was then diluted with water and extracted with EtOAc. The combined org. phases were dried over Na₂SO₄, filtered and concentrated in vacuo. Purification by flash chromatography (Hex/EtOAc 100:0 to 90:10) gave (5-methyl-2-nitro-phenyl)-propyl-amine (3.87 g, 99%). HPLC (System 3, 30-100% CH₃CN): t_{R}=3.477 min, MS (ES+): 195 [M+1].

### 3,4-Dimethyl-N-2-propyl-benzene-1,2-diamine

A solution of 2-fluoro-3,4-dimethyl-1-nitro-benzene (3.38 g, 20.0 mmol) and propylamine (5.0 ml, 60.0 mmol) in EtOH (5 ml) and water (1 ml) was heated to 150° C. for 30 min in a microwave oven. The mixture was then diluted with water and extracted with EtOAc. The combined org. phases were dried over Na₂SO₄, filtered and concentrated in vacuo. Purification by flash chromatography (Hex/EtOAc 100:0 to 90:10) gave (2,3-dimethyl-6-nitro-phenyl)-propyl-amine (4.08 g, 19.6 mmol) which was diluted in anhydrous THF (100 ml) and treated with Pd/C (10%, 500 mg). The mixture was stirred under H₂ at RT for 30 min. The mixture was then filtered and concentrated in vacuo. Purification by flash chromatography (Hex/EtOAc 100:0 to 80:20) provided the desired product (3.11 g, 91%). HPLC (System 3, 30-100% CH₃CN): t_{R}=0.398 min, MS (ES+): 179 [M+1].

### 3,5-Dimethyl-N-2-propyl-benzene-1,2-diamine

A solution of (2,4-dimethyl-6-nitro-phenyl)-propyl-amine (355 mg, 1.70 mmol) in THF (20 ml) was treated with Pd/C (10%, 50 mg) and the mixture was stirred under H₂ at RT for 18 h. The mixture was then filtered and concentrated in vacuo. Purification by flash chromatography (Hex/EtOAc 100:0 to 80:20) provided the desired product (217 mg, 71%). HPLC (System 2, 10-100% CH₃CN): t_{R}=2.419 min, MS (ES+): 179 [M+1].

The starting materials can be prepared as described hereafter:

### (2,4-Dimethyl-6-nitro-phenyl)-propyl-amine

A suspension of N-(2,4-dimethyl-6-nitro-phenyl)-N-propyl-acetamide (950 mg, 3.80 mmol) in water 1 ml) was treated dropwise with conc. H₂SO₄ (15 ml), and the mixture was then heated to 140° C. for 24 h. The mixture was then poured onto ice/water and extracted with EtOAc. The combined org. phases were dried over Na₂SO₄, filtered and concentrated in vacuo. Purification by flash chromatography (Hex/EtOAc 100:0 to 80:20) afforded (2,4-dimethyl-6-nitro-phenyl)-propyl-amine (355 mg, 45%). HPLC (System 4, 40-100% CH₃CN): t_{R}=3.149 min, MS (ES+): 209 [M+1].

### N-(2,4-Dimethyl-6-nitro-phenyl)-N-propyl-acetamide

A solution of N-(2,4-dimethyl-6-nitro-phenyl)-acetamide (2.0 g, 9.61 mmol) [F. Kanetani, H. Yamaguchi, Bull. Chem. Soc. Jpn. (1981), 54, 10, 3048-3058] in anhydrous THF (25 ml) was cooled to 0° C. and treated with NaH (60% in oil, 876 mg, 21.9 mmol). The mixture was allowed to warm to RT over 1 h, then cooled again to 0° C. and treated with 1-iodopropane (1.60 ml, 16.1 mmol). The solution was allowed to warm to RT and stirred for 18 h. The solution was then warm to 60° C. for 6 h and then diluted with EtOAc and washed with water. The combined org. phases were dried over Na₂SO₄, filtered and concentrated in vacuo. Purification by flash chromatography (Hex/EtOAc 100:0 to 75:25) gave N-(2,4-dimethyl-6-nitro-phenyl)-N-propyl-acetamide (951 mg, 40%). HPLC (System 3, 30-100% CH₃CN): t_{R}=2.700 min, MS (ES+): 251 [M+1].

### 4,5-Dimethyl-N-propyl-benzene-1,2-diamine

A solution of (4,5-dimethyl-2-nitro-phenyl)-propyl-amine (2.2 g, 10.6 mmol) in THF (75 ml) was treated with Pd/C (10%, 250 mg) and the mixture was stirred under H₂ at RT for 96 h. The mixture was then filtered and concentrated in vacuo. Purification by flash chromatography (Hex/EtOAc 100:0 to 80:20) provided the desired product (1.25 g, 66%). HPLC (System 2, 10-100% CH₃CN): t_{R}=2.421 min, MS (ES+): 179 [M+1].

The starting materials can be prepared as described hereafter:

### (4,5-Dimethyl-2-nitro-phenyl)-propyl-amine

A solution of 1-chloro-4,5-dimethyl-2-nitro-benzene (3.75 g, 20.0 mmol) and propylamine (5.0 ml, 60.0 mmol) in EtOH (5 ml) and water (1 ml) was heated to 150° C. for 4 h in a microwave oven. The mixture was then diluted with water and extracted with EtOAc. The combined org. phases were dried over Na₂SO₄, filtered and concentrated in vacuo. Purification by flash chromatography (Hex/EtOAc 100:0 to 90:10) gave (4,5-Dimethyl-2-nitro-phenyl)-propyl-amine (2.26 g, 54%). HPLC (System 3, 30-100% CH₃CN): t_{R}=3.650 min, MS (ES+): 209 [M+1].

### 3,6-Dimethyl-N-propyl-benzene-1,2-diamine

A solution of (3,6-dimethyl-2-nitro-phenyl)-propyl-amine (700 mg, 3.36 mmol) in THF (15 ml) was treated with Pd/C (10%, 100 mg) and the mixture was stirred under H₂ at RT for 72 h. The mixture was then filtered and concentrated in vacuo. Purification by flash chromatography (Hex/EtOAc 100:0 to 80:20) provided the desired product (508 mg, 85%). HPLC (System 2, 10-100% CH₃CN): t_{R}=2.430 min, MS (ES+): 179 [M+1].

The starting materials can be prepared as described hereafter:

### (3,6-Dimethyl-2-nitro-phenyl)-propyl-amine

A suspension of N-(3,6-dimethyl-2-nitro-phenyl)-N-propyl-acetamide (2.5 g, 9.99 mmol) in water (2 ml) was treated dropwise with conc. H₂SO₄ (20 ml), and the mixture was then heated to 140° C. for 48 h. The mixture was then poured onto ice/water and extracted with EtOAc. The combined org. phases were dried over Na₂SO₄, filtered and concentrated in vacuo. Purification by flash chromatography (Hex/EtOAc 100:0 to 50:50) afforded (3,6-dimethyl-2-nitro-phenyl)-propyl-amine (702 mg, 34%). HPLC (System 3, 30-100% CH₃CN): t_{R}=3.423 min, MS (ES⁺): 209 [M+1].

### N-(3,6-Dimethyl-2-nitro-phenyl)-N-propyl-acetamide

A solution of N-(3,6-dimethyl-2-nitro-phenyl)-acetamide (3.0 g, 14.4 mmol) [H. Suzuki, A. Tatsumi, T. Ishibashi, T. Mori, J. Chem. Soc. Perkin Trans. 1 (1985) 339-343] in anhydrous DMF (75 ml) was cooled to 0° C. and treated with NaH (60% in oil, 1.15 g, 28.8 mmol). The mixture was allowed to warm to RT over 1 h, then cooled again to 0° C. and treated with 1-iodopropane (4.30 ml, 43.2 mmol). The solution was allowed to warm to RT and stirred for 18 h. The solution was diluted with EtOAc and washed with water. The combined org. phases were dried over Na₂SO₄, filtered and concentrated in vacuo. Purification by flash chromatography (Hex/EtOAc 100:0 to 50:50) provided N-(3,6-dimethyl-2-nitro-phenyl)-N-propyl-acetamide (2.53 g, 70%). HPLC (System 3, 30-100% CH₃CN): t_{R}=2.579 min, MS (ES+): 251 [M+1].

### 4,5-Difluoro-N-propyl-benzene-1,2-diamine

A solution of (4,5-Difluoro-2-nitro-phenyl)-propyl-amine (1.0 g, 4.44 mmol) in anhydrous THF (50 ml) was treated with Pd/C (10%, 198 mg) and the mixture was stirred under H₂ at RT for 17 h. The mixture was then filtered and concentrated in vacuo. Purification by flash chromatography (Hex/EtOAc 100:0 to 75:25) provided the desired product (500 mg, 60%). UPLC (5-100% CH₃CN): t_{R}=0.898 min, TLC (Hex/EtOAc 1:1): R_{f}=0.65.

The starting material is prepared as described hereafter.

### (4,5-Difluoro-2-nitro-phenyl)-propyl-amine

A mixture of 1,2,4-trifluoro-5-nitro-benzene (2.0 g, 11.2 mmol), propylamine (1.31 ml, 15.7 mmol), K₂CO₃ (2.01 g, 14.5 mmol) in anhydrous THF (60 ml) was stirred at RT for 18 h. The mixture was then diluted with water and extracted with EtOAc. The combined org. layers were washed with brine, dried over Na₂SO₄, filtered and concentrated in vacuo. Purification by flash chromatography (Hex/EtOAc 100:0 to 90:10) furnished (4,5-difluoro-2-nitro-phenyl)-propyl-amine (1.03 g, 43%). UPLC (5-100% CH₃CN): t_{R}=1.669 min, TLC (Hex/EtOAc 9:1): R_{f}=0.53.

### 4-Fluoro-N-2-propyl-benzene-1,2-diamine

A solution of (5-fluoro-2-nitro-phenyl)-propyl-amine (4.6 g, 23.2 mmol) in anhydrous THF was treated with Pd/C (10%, 250 mg) and the solution was stirred under H₂ for 96 h at RT. The mixture was then filtered and concentrated in vacuo. Purification by flash chromatography (Hex/EtOAc 80:20) gave 4-fluoro-N-2-propyl-benzene-1,2-diamine (3.14 g, 80%). HPLC (System 2, 10-100% CH₃CN): t_{R}=2.394 min, MS (ES+): 169 [M+1].

The starting material is prepared as described hereafter

### (5-Fluoro-2-nitro-phenyl)-propyl-amine

A mixture of 2,4-difluoro-nitro-benzene (2.19 ml, 20.0 mmol), propylamine (2.33 ml, 27.9 mmol), and K₂CO₃ (3.59 g, 26.0 mmol) in anhydrous THF was stirred at RT for 18 h. The mixture was then diluted with water and extracted with EtOAc. The combined org. layers were dried over Na₂SO₄, filtered and concentrated in vacuo. Purification by flash chromatography (Hex/EtOAc 100:0 to 90:10) provided (5-fluoro-2-nitro-phenyl)-propyl-amine (4.67 g, quantitative). HPLC (System 3, 30-100% CH₃CN): t_{R}=3.291 min, MS (ES+): 199 [M+1].

### 3-Chloro-N-2-propyl-benzene-1,2-diamine

A solution of (2-chloro-6-nitro-phenyl)-propyl-amine (2.15 g, 10.0 mmol) in anhydrous THF was treated with Pt/C (5%, 0.1 g) and the mixture was stirred under H₂ for 8 h at RT. The mixture was then filtered on celite and concentrated in vacuo to give 3-chloro-N-2-propyl-benzene-1,2-diamine (1.8 g, 97%). UPLC (5-100% CH₃CN): t_{R}=0.976 min, MS (ES+): 185 [M+1].

The starting material is prepared as described hereafter

### (2-Chloro-6-nitro-phenyl)-propyl-amine

A solution of 1,2-dichloro-3-nitro-benzene (4.0 g, 20.8 mmol) in DMSO (20 ml) was treated with propylamine (7.2 ml, 87.0 mmol) and the mixture was heated to 100° C. for 90 min. The solution was diluted with water and extracted with EtOAc. The combined org. layers were washed with water, dried over Na₂SO₄, filtered, and concentrated in vacuo to give (2-chloro-6-nitro-phenyl)-propyl-amine (4.3 g, 96%). UPLC (5-100% CH₃CN): t_{R}=1.727 min, MS (ES+): 215 [M+1].

### 3-Fluoro-N-2-propyl-benzene-1,2-diamine

A solution of (2-fluoro-6-nitro-phenyl)-propyl-amine (1.90 g, 9.59 mmol) in anhydrous THF (25 ml) was treated with Pd/C (10%, 250 mg) and the solution was stirred under H₂ for 96 h at RT. The mixture was then filtered on celite and concentrated in vacuo. Purification by flash chromatography (Hex/EtOAc 100:0 to 80:20) provided 3-fluoro-N-2-propyl-benzene-1,2-diamine (791 mg, 49%). HPLC (System 2, 10-100% CH₃CN): t_{R}=1.683 min, TLC (Hex/EtOAc 4:1): R_{f}=0.38.

The starting material is prepared as described hereafter

### (2-Fluoro-6-nitro-phenyl)-propyl-amine

A solution of 2-fluoro-6-nitro-phenol (1.00 g, 6.37 mmol) in acetone (18 ml) was treated with K₂CO₃ (880 mg, 6.37 mmol), stirred for 20 min at RT, and trifluoromethanesulfonic anhydride (1.07 ml, 6.36 mmol) was then added dropwise. The mixture was stirred for 4 h at RT, diluted with Et₂O, washed with an aq. 0.1N NaOH solution, dried over Na₂SO₄, filtered, and concentrated in vacuo to give trifluoro-methanesulfonic acid 2-fluoro-6-nitro-phenyl ester (0.75 g, ca. 40%) as brownish liquid which was used as it is for the next step. A solution of trifluoro-methanesulfonic acid 2-fluoro-6-nitro-phenyl ester (12.04 g, 38.7 mmol) in NMP (20 ml) was treated with propylamine (3.55 ml, 42.6 mmol) and heated to 130° C. for 18 h. The mixture was allowed to cool to RT, diluted with Et₂O, and washed twice with water. The org. layer was then concentrated in vacuo to give a dark brown liquid, which was purified by flash chromatography (Hex/EtOAc 90:10) providing (2-fluoro-6-nitro-phenyl)-propyl-amine (1.94 g, 25%). HPLC (System 3, 30-100% CH₃CN): t_{R}=3.417 min, MS (LC-MS): 199 [M+1].

### 3-Chloro-5-iodo-N-2-propyl-benzene-1,2-diamine

A solution of (2-chloro-4-iodo-6-nitro-phenyl)-propyl-amine (1.60 g, 4.70 mmol) in EtOH (15 ml) was treated with SnCl₂.2H₂O (2.16 g, 9.38 mmol) and the mixture was heated to reflux for 90 min. The mixture was then allowed to cool to RT, diluted with EtOAc and washed with an aq. 2N NaOH solution and water. The aqueous phase was extracted with EtOAc, and the combined org. layers were then dried over Na₂SO₄, filtered, and concentrated in vacuo. Purification by flash chromatography (Hex/EtOAc 100:0 to 80:20) furnished the desired product as a brown oil (1.37 g, 94%). HPLC (System 3, 30-100% CH₃CN): t_{R}=3.087 min, TLC (Hex/EtOAc 4:1): R_{f}=0.40.

The starting materials are prepared as described below:

### (2-Chloro-4-iodo-6-nitro-phenyl)-propyl-amine

A suspension of Ag₂SO₄ (1.44 g, 4.61 mmol) and iodine (1.18 g, 4.63 mmol) in EtOH (30 ml) was stirred at RT for 15 min, then treated with (2-chloro-6-nitro-phenyl)-propyl-amine (1.00 g, 4.61 mmol) and stirred for another 2 h. The mixture was then filtered and the filtrated was concentrated in vacuo. The residue was taken up in EtOAc, washed with a 10% aq. Na₂S₂O₃ solution and water. The water phase was extracted with EtOAc and the combined org. layers were then dried over Na₂SO₄, filtered, and concentrated in vacuo. Purification by flash chromatography (Hex/EtOAc 95:5) gave (2-chloro-4-iodo-6-nitro-phenyl)-propyl-amine (1.65 g, quantitative). HPLC (System 4, 40-100% CH₃CN): t_{R}=3.821 min, TLC (Hex/EtOAc 19:1): R_{f}=0.58.

### (2-Chloro-6-nitro-phenyl)-propyl-amine

A solution of 1,2-dichloro-3-nitro-benzene (38.8 g, 200 mmol) in N,N-dimethylacetamide (100 ml) was cooled to 0° C. and propylamine (70.0 ml, 839 mmol) was added dropwise. The mixture was then heated to 100° C. for 3 h. The mixture was then allowed to cool to RT, diluted with EtOAc, and washed with water. The org. layer was then dried over Na₂SO₄, filtered, and concentrated in vacuo. Purification by distillation (0.1 Torr, 103-107° C.) gave (2-chloro-6-nitro-phenyl)-propyl-amine (39.9 g, 93%). HPLC (System 4, 40-100% CH₃CN): t_{R}=3.074 min, TLC (Hex/EtOAc 4:1): R_{f}=0.64.

### EXAMPLE 1

### [3-Chloro-5-(1-methyl-1H-benzoimidazol-2-yl)-pyridin-2-yl]-(4-chloro-phenyl)-amine

A mixture of [5-(1H-Benzoimidazol-2-yl)-3-chloro-pyridin-2-yl]-(4-chloro-phenyl)-amine (160 mg, 0.45 mmol) in anhydrous DMF (4 ml) was treated with NaH (13 mg, 0.49 mmol) and the solution was stirred at RT for 30 min, prior to adding iodomethane (57 μl, 0.90 mmol). The stirring was continued for 1 h and the reaction was quenched by adding water. The mixture was extracted with EtOAc, and the combined org layers were washed with brine, dried over Na₂SO₄, filtered, and concentrated in vacuo to give a yellow-brownish solid which was purified by flash chromatography (DCM/MeOH 100:0 to 95:5) to provide the desired product (89 mg, 54%). UPLC (5-100% CH₃CN): t_{R}=1.218 min, TLC (DCM/MeOH 9:1): R_{f}=0.90.

The starting materials are prepared as described below:

### [5-(1H-Benzoimidazol-2-yl)-3-chloro-pyridin-2-yl]-(4-chloro-phenyl)-amine

A mixture of 5-chloro-6-(4-chloro-phenylamino)-nicotinic acid (454 mg, 1.60 mmol) and 1,2-phenylenediamine (210 mg, 1.92 mmol) in PPA (5 ml) was heated to 210° C. in a microwave oven for 5 min. The mixture was poured onto water, rendered basic to pH 8 with an aq. soln. of 2N NaOH, and extracted with EtOAc. The combined org. layers were then dried and concentrated in vacuo and the crude product was purified by flash chromatography (Hex/EtOAc 80:20 to 60:40) to give [5-(1H-benzoimidazol-2-yl)-3-chloro-pyridin-2-yl]-(4-chloro-phenyl)-amine (348 mg, 61%). UPLC (5-100% CH₃CN): t_{R}=1.212 min, TLC (Hex/EtOAc 1:1): R_{f}=0.68.

### 5-Chloro-6-(4-chloro-phenylamino)-nicotinic Acid

A mixture of 5,6-dichloronicotinic acid (4.0 g, 20.8 mmol) and 4-chloroaniline (3.22 g, 25.0 mmol) in acetic acid (20 ml) was heated to 150° C. in a microwave oven for 75 min. After cooling to RT, the precipitate was filtered off. The filtrate was then treated with EtOAc and another precipitate formed, which was filtered. Purification by re-crystallization from 2-PrOH gave 5-chloro-6-(4-chloro-phenylamino)-nicotinic acid (1.77 g, 30%). UPLC (5-100% CH₃CN): t_{R}=1.426 min, TLC (DCM/MeOH 9:1): R_{f}=0.42.

Following the same sequence, the following compounds can be prepared:

### EXAMPLE 2

### [3-Chloro-5-(1-ethyl-1H-benzoimidazol-2-yl)-pyridin-2-yl]-(4-chloro-phenyl)-amine; UPLC (5-100% CH₃CN): t_{R}=1.285 min; TLC (Hex/EtOAc 3:2): R_{f}=0.56

### EXAMPLE 3

### (4-Chloro-phenyl)-[3-chloro-5-(1-propyl-1H-benzoimidazol-2-yl)-pyridin-2-yl]-amine; UPLC (5-100% CH₃CN): t_{R}=1.370 min; TLC (Hex/EtOAc 3:2): R_{f}=0.57

### EXAMPLE 4

### [5-(1-Butyl-1H-benzoimidazol-2-yl)-3-chloro-pyridin-2-yl]-(4-chloro-phenyl)-amine; UPLC (5-100% CH₃CN): t_{R}=1.441 min; TLC (Hex/EtOAc 3:2): R_{f}=0.62

### EXAMPLE 5

### [3-Chloro-5-(1-isopropyl-1H-benzoimidazol-2-yl)-pyridin-2-yl]-(4-chloro-phenyl)-amine; UPLC (System 3, 30-100% CH₃CN): t_{R}=2.706 min; TLC (Hex/EtOAc 3:2): R_{f}=0.51

### EXAMPLE 6

### [3-Chloro-5-(1-isobutyl-1H-benzoimidazol-2-yl)-pyridin-2-yl]-(4-chloro-phenyl)-amine; UPLC (5-100% CH₃CN): t_{R}=1.425 min; TLC (Hex/EtOAc 3:2): R_{f}=0.58

### EXAMPLE 7

### [3-Chloro-5-(1-cyclopropylmethyl-1H-benzoimidazol-2-yl)-pyridin-2-yl]-(4-chloro-phenyl)-amine; UPLC (5-100% CH₃CN): t_{R}=1.379 min; TLC (Hex/EtOAc 3:2): R_{f}=0.43

### EXAMPLE 8

### (4-Chloro-phenyl)-[3-chloro-5-(1-propyl-1H-imidazo[4,5-c]pyridin-2-yl)-pyridin-2-yl]-amine; HPLC (System 2, 10-100% CH₃CN): t_{R}=3.167 min; MS (LC-MS): 399 [M+1]

[3-Chloro-5-(1H-imidazo[4,5-c]pyridin-2-yl)-pyridin-2-yl]-(4-chloro-phenyl)-amine can be pepared according to the procedure described above using 5-chloro-6-(4-chloro-phenylamino)-nicotinic acid and 2-3-diaminopyridine. HPLC (System 1, 0-100% CH₃CN): t_{R}=2.976 min, TLC (DCM/MeOH 9:1): R_{f}=0.43.

### EXAMPLE 9

### [3-Chloro-5-(5-fluoro-1-methyl-1H-benzoimidazol-2-yl)-pyridin-2-yl]-(4-chloro-phenyl)-amine

A mixture of [3-Chloro-5-(5-fluoro-1H-benzoimidazol-2-yl)-pyridin-2-yl]-(4-chloro-phenyl)-amine (200 mg, 0.54 mmol) in anhydrous DMF (4 ml) was treated with NaH (13 mg, 0.49 mmol) and the solution was stirred at RT for 30 min, prior to adding iodomethane (56 μl, 0.90 mmol). The stirring was continued for 3 h and the reaction was quenched by adding water. The mixture was extracted with EtOAc, and the combined org layers were washed with brine, dried over Na₂SO₄, filtered, and concentrated in vacuo to give a yellow-brownish solid which was purified by flash chromatography (DCM/MeOH 100:0 to 90:10) to provide [3-chloro-5-(5-fluoro-1-methyl-1H-benzoimidazol-2-yl)-pyridin-2-yl]-(4-chloro-phenyl)-amine (73 mg, 35%). UPLC (5-100% CH₃CN): t_{R}=1.255 min, TLC (Tol/EtOAc 5:1): R_{f}=0.45.

The starting material is prepared as described below:

A mixture of 5-chloro-6-(4-chloro-phenylamino)-nicotinic acid (450 mg, 1.59 mmol) and 4-fluoro-1,2-phenylenediamine (241 mg, 1.91 mmol) in PPA (5 ml) was heated to 210° C. in a microwave oven for 10 min. The mixture was poured onto water, stirred overnight, rendered basic to pH 8 with an aq. soln. of 2N NaOH, and extracted with EtOAc. The combined org. layers were washed with brine, dried, concentrated in vacuo, and the crude product was purified by flash chromatography (Hex/EtOAc 100:0 to 60:40) to give [3-chloro-5-(5-fluoro-1H-benzoimidazol-2-yl)-pyridin-2-yl]-(4-chloro-phenyl)-amine (443 mg, 75%) UPLC (5-100% CH₃CN): t_{R}=1.256 min, TLC (Hex/EtOAc 1:1): R_{f}=0.75.

During the purification of Example 8, the following compound can also be isolated:

### EXAMPLE 10

### [3-Chloro-5-(6-fluoro-1-methyl-1H-benzoimidazol-2-yl)-pyridin-2-yl]-(4-chloro-phenyl)-amine; UPLC (5-100% CH₃CN): t_{R}=1.265 min; TLC (Tol/EtOAc 5:1): R_{f}=0.40

By analogy with examples 8 and 9, the following compounds can be synthesized:

### EXAMPLE 11

### [3-Chloro-5-(1-ethyl-5-fluoro-1H-benzoimidazol-2-yl)-pyridin-2-yl]-(4-chloro-phenyl)-amine; UPLC (5-100% CH₃CN): t_{R}=1.322 min; TLC (Tol/EtOAc 5:1): R_{f}=0.48

### EXAMPLE 12

### [3-Chloro-5-(1-ethyl-6-fluoro-1H-benzoimidazol-2-yl)-pyridin-2-yl]-(4-chloro-phenyl)-amine; UPLC (5-100% CH₃CN): t_{R}=1.335 min; TLC (Tol/EtOAc 5:1): R_{f}=0.45

### EXAMPLE 13

### [3-Chloro-5-(5-fluoro-1-propyl-1H-benzoimidazol-2-yl)-pyridin-2-yl]-(4-chloro-phenyl)-amine; UPLC (5-100% CH₃CN): t_{R}=1.412 min; TLC (Hex/EtOAc 4:1): R_{f}=0.27

### EXAMPLE 14

### [3-Chloro-5-(6-fluoro-1-propyl-1H-benzoimidazol-2-yl)-pyridin-2-yl]-(4-chloro-phenyl)-amine; UPLC (5-100% CH₃CN): t_{R}=1.424 min; TLC (Hex/EtOAc 4:1): R_{f}=0.24

### EXAMPLE 15

### [(4-Chloro-phenyl)-[3-chloro-5-(3-propyl-3H-imidazo[4,5-b]pyridin-2-yl)-pyridin-2-yl]-amine; HPLC (System 1, 0-100% CH₃CN): t_{R}=3.825 min; TLC (Hex/EtOAc 9:1): R_{f}=0.52

### EXAMPLE 16

### [3-Chloro-5-(1-ethyl-1H-benzoimidazol-2-yl)-pyridin-2-yl]-(6-methyl-pyridin-3-yl)-amine

A mixture of 5-chloro-6-(6-methyl-pyridin-3-ylamino)-nicotinic acid (200 mg, 0.76 mmol) and N-ethyl-benzene-1,2-diamine (124 mg, 0.91 mmol) in PPA (3 ml) was heated to 210° C. in a microwave oven for 8 min. The mixture was then poured onto water and stirred at RT for 18 h. The pH of the solution was adjusted to 8 with an aq. soln. of 2N NaOH, and the mixture was extracted with EtOAc. The combined org. layers were dried over Na₂SO₄, filtered and concentrated in vacuo. Purification by flash chromatography (Hex/EtOAc 100:0 to 0:100), followed by crystallization from Hex gave the desired product (107 mg, 39%). HPLC (System 2, 10-100% CH₃CN): t_{R}=1.017 min, MS (ES+): 364 [M+1]

The starting materials can be prepared as described hereafter:

### 5-Chloro-6-(6-methyl-pyridin-3-ylamino)-nicotinic Acid

A solution of 5-chloro-6-(6-methyl-pyridin-3-ylamino)-nicotinic acid methyl ester (42.0 g, 151 mmol) in MeOH (500 ml) was treated slowly with an aq. soln. of 1N NaOH (300 ml, 300 mmol). The solution was stirred for 1 h at RT and the mixture was then neutralized by adding 4N aq. HCl. 5-Chloro-6-(6-methyl-pyridin-3-ylamino)-nicotinic acid precipitated and was collected by filtration (38.0 g, 95%). UPLC (5-100% CH₃CN): t_{R}=0.647 min, MS (ES+): 264 [M+1]

### 5-Chloro-6-(6-methyl-pyridin-3-ylamino)-nicotinic Acid Methyl Ester

A suspension of 5,6-dichloro-nicotinic acid methyl ester (50.0 g, 243 mmol), 3-amino-6-methylpyridine (40.2 g, 364 mmol), rac-BINAP (9.05 g, 14.5 mmol), Pd₂(dba)₃ (11.1 g, 12.1 mmol) and K₂CO₃ (101.0 g, 731 mmol) in toluene was heated to 120° C. for 16 h. The mixture was allowed to cool to RT and concentrated in vacuo. Purification by re-crystallization in Tol/EtOAc provided 5-chloro-6-(6-methyl-pyridin-3-ylamino)-nicotinic acid methyl ester (37.8 g, 56%). UPLC (5-100% CH₃CN): t_{R}=0.832 min, MS (ES+): 278 [M+1]

### 5,6-Dichloro-nicotinic Acid Methyl Ester

A solution of 5,6-dichloro-nicotinic acid (55.0 g, 281 mmol) in SOCl₂ (204 ml) was treated with DMF (0.1 ml) and the mixture was heated to 80° C. for 5 h. The excess of SOCl₂ was evaporated and the crude product was taken up in MeOH (300 ml) and the resulting solution was heated to reflux for 1 h. The mixture was then allowed to cool slowly to RT. 5,6-Dichloro-nicotinic acid methyl ester precipitated and was collected by filtration (55.6 g, 96%). UPLC (5-100% CH₃CN): t_{R}=1.384 min, TLC (Hex/EtOAc 1:1): R_{f}=0.76.

By analogy to the preparation of example 16, the following compounds can be made:

### EXAMPLE 17

### [3-Chloro-5-(1-propyl-1H-benzoimidazol-2-yl)-pyridin-2-yl]-(6-methyl-pyridin-3-yl)-amine; UPLC (5-100% CH₃CN): t_{R}=0.810 min; TLC (Hex/EtOAc 3:1): R_{f}=0.15

### EXAMPLE 18

### [5-(1-Butyl-1H-benzoimidazol-2-yl)-3-chloro-pyridin-2-yl]-(6-methyl-pyridin-3-yl)-amine; HPLC (System 2, 10-100% CH₃CN): t_{R}=2.068 min; MS (ES+): 392 [M+1]

### EXAMPLE 19

### [3-Chloro-5-(1-pentyl-1H-benzoimidazol-2-yl)-pyridin-2-yl]-(6-methyl-pyridin-3-yl)-amine; HPLC (System 2, 10-100% CH₃CN): t_{R}=2.679 min; MS (ES+): 406 [M+1]

### EXAMPLE 20

### [3-Chloro-5-(1-isobutyl-1H-benzoimidazol-2-yl)-pyridin-2-yl]-(6-methyl-pyridin-3-yl)-amine; HPLC (System 2, 10-100% CH₃CN): t_{R}=2.544 min; MS (ES+): 392 [M+1]

### EXAMPLE 21

### [3-Chloro-5-(1-propyl-5-trifluoromethyl-1H-benzoimidazol-2-yl)-pyridin-2-yl]-(6-methyl-pyridin-3-yl)-amine; HPLC (System 2, 10-100% CH₃CN): t_{R}=3.094 min; MS (ES+): 446 [M+1]

### EXAMPLE 22

### [3-Chloro-5-(5-methyl-1-propyl-1H-benzoimidazol-2-yl)-pyridin-2-yl]-(6-methyl-pyridin-3-yl)-amine; HPLC (System 2, 10-100% CH₃CN): t_{R}=2.578 min; MS (ES+): 392 [M+1]

### EXAMPLE 23

### [3-Chloro-5-(6-methyl-1-propyl-1H-benzoimidazol-2-yl)-pyridin-2-yl]-(6-methyl-pyridin-3-yl)-amine; HPLC (System 2, 10-100% CH₃CN): t_{R}=2.582 min; MS (ES+): 392 [M+1]

### EXAMPLE 24

### [3-Chloro-5-(7-methyl-1-propyl-1H-benzoimidazol-2-yl)-pyridin-2-yl]-(6-methyl-pyridin-3-yl)-amine; HPLC (System 2, 10-100% CH₃CN): t_{R}=2.509 min; MS (ES+): 392 [M+1]

### EXAMPLE 25

### [3-Chloro-5-(4-methyl-1-propyl-1H-benzoimidazol-2-yl)-pyridin-2-yl]-(6-methyl-pyridin-3-yl)-amine; HPLC (System 2, 10-100% CH₃CN): t_{R}=2.542 min; MS (ES+): 392 [M+1]

### EXAMPLE 26

### [3-Chloro-5-(6,7-dimethyl-1-propyl-1H-benzoimidazol-2-yl)-pyridin-2-yl]-(6-methyl-pyridin-3-yl)-amine; HPLC (System 2, 10-100% CH₃CN): t_{R}=2.667 min; MS (ES+): 406 [M+1]

### EXAMPLE 27

### [3-Chloro-5-(5,7-dimethyl-1-propyl-1H-benzoimidazol-2-yl)-pyridin-2-yl]-(6-methyl-pyridin-3-yl)-amine; HPLC (System 2, 10-100% CH₃CN): t_{R}=2.667 min; MS (ES+): 406 [M+1]

### EXAMPLE 28

### [3-Chloro-5-(5,6-dimethyl-1-propyl-1H-benzoimidazol-2-yl)-pyridin-2-yl]-(6-methyl-pyridin-3-yl)-amine; HPLC (System 2, 10-100% CH₃CN): t_{R}=2.630 min; MS (ES+): 406 [M+1]

### EXAMPLE 29

### [3-Chloro-5-(4,7-dimethyl-1-propyl-1H-benzoimidazol-2-yl)-pyridin-2-yl]-(6-methyl-pyridin-3-yl)-amine; HPLC (System 2, 10-100% CH₃CN): t_{R}=2.638 min; MS (ES+): 406 [M+1]

### EXAMPLE 30

### [3-Chloro-5-(5,6-difluoro-1-propyl-1H-benzoimidazol-2-yl)-pyridin-2-yl]-(6-methyl-pyridin-3-yl)-amine; UPLC (5-100% CH₃CN): t_{R}=1.014 min; TLC (DCM/MeOH 9:1): R_{f}=0.72

### EXAMPLE 31

### [3-Chloro-5-(5,6-dihydro-4H-imidazo[4,5,1-ij]quinolin-2-yl)-pyridin-2-yl]-(6-methyl-pyridin-3-yl)-amine; HPLC (System 2, 10-100% CH₃CN): t_{R}=2.306 min; MS (LC-MS): 376 [M+1]

### EXAMPLE 32

### [3-Chloro-5-(1-propyl-1H-benzoimidazol-2-yl)-pyridin-2-yl]-phenyl-amine was prepared in analogy to the procedure described in example 16 starting from 5-Chloro-6-phenylamino-nicotinic acid and N-ethyl-benzene-1,2-diamine.

HPLC (System 2, 10-100% CH₃CN): t_{R}=3.199 min, MS (LC-MS): 363 [M+1].

The starting materials can be prepared in analogy to the protocols given for the starting materials of example 16.

5-Chloro-6-phenylamino-nicotinic acid; HPLC (System 3, 30-100% CH₃CN): t_{R}=2.581 min, MS (LC-MS): 263 [M+1].

5-Chloro-6-phenylamino-nicotinic acid methyl ester; HPLC (System 3, 30-100% CH₃CN): t_{R}=3.464 min, MS (LC-MS): 277 [M+1].

By analogy to the preparation of example 32, the following compounds can be made:

### EXAMPLE 33

### [3-Chloro-5-(1-propyl-1H-benzoimidazol-2-yl)-pyridin-2-yl]-pyridin-4-yl-amine was prepared in analogy to the procedure described in example 16 starting from 5-Chloro-6-(pyridin-4-ylamino)-nicotinic acid and N-ethyl-benzene-1,2-diamine

HPLC (System 2, 10-100% CH₃CN): t_{R}=2.353 min, MS (LC-MS): 364 [M+1].

The starting materials can be prepared in analogy to the protocols given for the starting materials of example 16.

5-Chloro-6-(pyridin-4-ylamino)-nicotinic acid; HPLC (System 2, 10-100% CH₃CN): t_{R}=0.747 min, MS (LC-MS): 250 [M+1].

5-Chloro-6-(pyridin-4-ylamino)-nicotinic acid methyl ester; HPLC (System 3, 30-100% CH₃CN): t_{R}=0.394 min, MS (LC-MS): 264 [M+1].

### EXAMPLE 34

### [3-Chloro-5-(1-propyl-1H-benzoimidazol-2-yl)-pyridin-2-yl]-p-tolyl-amine was prepared in analogy to the procedure described in example 16 starting from 5-Chloro-6-p-tolylamino-nicotinic acid and N-ethyl-benzene-1,2-diamine.

HPLC (System 2, 10-100% CH₃CN): t_{R}=3.331 min, MS (LC-MS): 377 [M+1].

The starting materials can be prepared in analogy to the protocols given for the starting materials of example 16.

5-Chloro-6-p-tolylamino-nicotinic acid; HPLC (System 3, 30-100% CH₃CN): t_{R}=2.581 min, MS (LC-MS): 263 [M+1].

5-Chloro-6-p-tolylamino-nicotinic acid methyl ester; HPLC (System 3, 30-100% CH₃CN): t_{R}=3.464 min, MS (LC-MS): 277 [M+1].

### EXAMPLE 34b

### [3-Chloro-5-(7-chloro-5-iodo-1-propyl-1H-benzoimidazol-2-yl)-pyridin-2-yl]-(6-methyl-pyridin-3-yl)-amine

A solution of 5-chloro-6-(6-methyl-pyridin-3-ylamino)-nicotinic acid (2.00 g, 7.58 mmol) in SOCl₂ (20 ml) was heated to 90° C. for 2 h. The mixture was then allowed to cool to RT, and then concentrated in vacuo. The residue was taken up in Hex and the suspension was filtered and dried in vacuo to give 5-chloro-6-(6-methyl-pyridin-3-ylamino)-nicotinoyl chloride which was used as it is in the next step. A solution of this acid chloride (1.06 g, 3.68 mmol) and 3-chloro-5-iodo-N-2-propyl-benzene-1,2-diamine (1.30 g, 4.19 mmol) in THF (50 ml) was heated to 70° C. for 1 h. The mixture was then allowed to cool to RT, and then diluted with EtOAc and washed several time with a sat. aq. solution of NaHCO₃. The org. phase was then dried over over Na₂SO₄, filtered, and concentrated in vacuo. The residue was taken up in Tol (100 ml), treated with p-toluenesulfonic acid (1.00 g, 5.18 mmol), and heated to reflux for 18 h. The solution was then allowed to cool to RT, diluted with EtOAc, and washed with a sat. aq. NaHCO₃ solution. The org. layer was then dried over over Na₂SO₄, filtered, and concentrated in vacuo. Purification by flash chromatography (Hex/EtOAc 100:0 to 0:100) and re-crystallization from Hex/EtOAc provided [3-chloro-5-(7-chloro-5-iodo-1-propyl-1H-benzoimidazol-2-yl)-pyridin-2-yl]-(6-methyl-pyridin-3-yl)-amine (938 mg, 44%). HPLC (System 3, 30-100% CH₃CN): t_{R}=2.898 min, TLC (EtOAc): R_{f}=0.64.

### EXAMPLE 35

### [3-Chloro-5-(1-ethyl-5-fluoro-1H-benzoimidazol-2-yl)-pyridin-2-yl]-(6-methyl-pyridin-3-yl)-amine

A solution of [3-chloro-5-(5-fluoro-1H-benzoimidazol-2-yl)-pyridin-2-yl]-(6-methyl-pyridin-3-yl)-amine (200 mg, 0.57 mmol) in DMF (4 ml) was treated with NaH (20.0 mg, 0.79 mmol) and the solution was stirred at RT for 30 min, prior to adding iodoethane (182 μl, 2.26 mmol). The stirring was continued for 18 h and the reaction was then quenched by adding a sat. aq. NaHCO₃ solution to adjust the pH to 9. The mixture was extracted with EtOAc, and the combined org layers were washed with brine, dried over Na₂SO₄, filtered, and concentrated in vacuo to give a yellow-brownish solid which was purified by flash chromatography (DCM/MeOH 100:0 to 90:10) and preparative TLC (EtOAc) to provide [3-chloro-5-(1-ethyl-5-fluoro-1H-benzoimidazol-2-yl)-pyridin-2-yl]-(6-methyl-pyridin-3-yl)-amine (35 mg, 16%). UPLC (5-100% CH₃CN): t_{R}=0.797 min, TLC (EtOAc): R_{f}=0.30.

The starting material is prepared as described below:

### [3-chloro-5-(5-fluoro-1H-benzoimidazol-2-yl)-pyridin-2-yl]-(6-methyl-pyridin-3-yl)-amine

A mixture of 5-chloro-6-(6-methyl-pyridin-3-ylamino)-nicotinic acid (1.0 g, 3.79 mmol) and 4-fluoro-1,2-phenylenediamine (574 mg, 4.55 mmol) in PPA (15 ml) was heated to 210° C. in a microwave oven for 35 min. The mixture was poured onto cold water, stirred overnight, rendered basic to pH 8 with an aq. soln. of 2N NaOH, and extracted with EtOAc. The combined org. layers were washed with brine, dried, concentrated in vacuo, and the crude product was purified by flash chromatography (DCM/MeOH 100:0 to 90:10) and re-crystallization from MeOH to afford [3-chloro-5-(5-fluoro-1H-benzoimidazol-2-yl)-pyridin-2-yl]-(6-methyl-pyridin-3-yl)-amine (877 mg, 65%). UPLC (5-100% CH₃CN): t_{R}=0.768 min, TLC (DCM/MeOH 9:1): R_{f}=0.57.

During the purification of Example 35, the following compound can also be isolated:

### EXAMPLE 36

### [3-Chloro-5-(1-ethyl-6-fluoro-1H-benzoimidazol-2-yl)-pyridin-2-yl]-(6-methyl-pyridin-3-yl)-amine; UPLC (5-100% CH₃CN): t_{R}=0.812 min; TLC (EtOAc): R_{f}=0.22

By analogy with examples 35 and 36, the following compounds can be synthesized:

### EXAMPLE 37

### [3-Chloro-5-(5-fluoro-1-propyl-1H-benzoimidazol-2-yl)-pyridin-2-yl]-(6-methyl-pyridin-3-yl)-amine; UPLC (5-100% CH₃CN): t_{R}=0.881 min; TLC (EtOAc): R_{f}=0.32

### EXAMPLE 38

### [3-Chloro-5-(6-fluoro-1-propyl-1H-benzoimidazol-2-yl)-pyridin-2-yl]-(6-methyl-pyridin-3-yl)-amine; UPLC (5-100% CH₃CN): t_{R}=0.893 min; TLC (EtOAc): R_{f}=0.23

### EXAMPLE 39

### [5-(1-Butyl-5-fluoro-1H-benzoimidazol-2-yl)-3-chloro-pyridin-2-yl]-(6-methyl-pyridin-3-yl)-amine; UPLC (5-100% CH₃CN): t_{R}=0.969 min; TLC (EtOAc): R_{f}=0.33

### EXAMPLE 40

### [5-(1-Butyl-6-fluoro-1H-benzoimidazol-2-yl)-3-chloro-pyridin-2-yl]-(6-methyl-pyridin-3-yl)-amine; UPLC (5-100% CH₃CN): t_{R}=0.981 min; TLC (EtOAc): R_{f}=0.24

### EXAMPLE 41

### [3-Chloro-5-(4-fluoro-1-propyl-1H-benzoimidazol-2-yl)-pyridin-2-yl]-(6-methyl-pyridin-3-yl)-amine; UPLC (5-100% CH₃CN): t_{R}=1.011 min; TLC (EtOAc): R_{f}=0.39

### EXAMPLE 42

### [3-Chloro-5-(7-fluoro-1-propyl-1H-benzoimidazol-2-yl)-pyridin-2-yl]-(6-methyl-pyridin-3-yl)-amine; UPLC (5-100% CH₃CN): t_{R}=0.985 min; TLC (EtOAc): R_{f}=0.27

### EXAMPLE 43

### [3-Chloro-5-(4,5-difluoro-1-propyl-1H-benzoimidazol-2-yl)-pyridin-2-yl]-(6-methyl-pyridin-3-yl)-amine; UPLC (5-100% CH₃CN): t_{R}=1.146 min; TLC (EtOAc): R_{f}=0.34

### EXAMPLE 44

### [3-Chloro-5-(6,7-difluoro-1-propyl-1H-benzoimidazol-2-yl)-pyridin-2-yl]-(6-methyl-pyridin-3-yl)-amine; UPLC (5-100% CH₃CN): t_{R}=1.147 min; TLC (EtOAc): R_{f}=0.24

### EXAMPLE 45

### [3-Chloro-5-(5-chloro-1-propyl-1H-benzoimidazol-2-yl)-pyridin-2-yl]-(6-methyl-pyridin-3-yl)-amine; UPLC (5-100% CH₃CN): t_{R}=1.006 min; MS (ES+): 412 [M+1]

### EXAMPLE 46

### [3-Chloro-5-(6-chloro-1-propyl-1H-benzoimidazol-2-yl)-pyridin-2-yl]-(6-methyl-pyridin-3-yl)-amine; UPLC (5-100% CH₃CN): t_{R}=1.005 min; MS (ES+): 412 [M+1]

### EXAMPLE 47

### [3-Chloro-5-(4-chloro-1-propyl-1H-benzoimidazol-2-yl)-pyridin-2-yl]-(6-methyl-pyridin-3-yl)-amine; UPLC (5-100% CH₃CN): t_{R}=1.055 min; MS (ES+): 412 [M+1]

### EXAMPLE 48

### [3-Chloro-5-(7-chloro-1-propyl-1H-benzoimidazol-2-yl)-pyridin-2-yl]-(6-methyl-pyridin-3-yl)-amine; UPLC (5-100% CH₃CN): t_{R}=1.076 min; TLC (EtOAc/Hex 4:1): R_{f}=0.30

### EXAMPLE 49

### [3-Chloro-5-(4,6-dichloro-1-propyl-1H-benzoimidazol-2-yl)-pyridin-2-yl]-(6-methyl-pyridin-3-yl)-amine; UPLC (5-100% CH₃CN): t_{R}=1.334 min; MS (ES+): 446 [M+1]

### EXAMPLE 50

### [3-Chloro-5-(5,7-dichloro-1-propyl-1H-benzoimidazol-2-yl)-pyridin-2-yl]-(6-methyl-pyridin-3-yl)-amine; UPLC (5-100% CH₃CN): t_{R}=1.393 min; MS (ES+): 446 [M+1]

### EXAMPLE 51

### [3-Chloro-5-(5,6-dichloro-1-propyl-1H-benzoimidazol-2-yl)-pyridin-2-yl]-(6-methyl-pyridin-3-yl)-amine; UPLC (5-100% CH₃CN): t_{R}=1.244 min; MS (ES+): 446 [M+1]

### EXAMPLE 52

### [3-Chloro-5-(7-chloro-1-propyl-1H-benzoimidazol-2-yl)-pyridin-2-yl]-(4-chloro-phenyl)-amine

A mixture of 5-chloro-6-(4-chloro-phenylamino)-nicotinic acid (5.0 g, 17.7 mmol) and 3-chloro-N-2-propyl-benzene-1,2-diamine (3.26 g, 17.7 mmol) in PPA (5 ml) was heated to 200° C. for 18 h. The mixture was then poured onto water and stirred at RT for 4 h. The pH of the solution was adjusted to 8 with a 20% aq. soln. of NaOH, and the mixture was extracted with EtOAc. The combined org. layers were dried over Na₂SO₄, filtered and concentrated in vacuo. Purification by flash chromatography (Hex/EtOAc 100:0 to 80:20), followed by re-crystallization from Hex/EtOAc gave the desired product (1.17 g, 15%). HPLC (System 3, 30-100% CH₃CN): t_{R}=3.535 min, TLC (Hex/EtOAc 3:1): R_{f}=0.41.

### EXAMPLE 53

### [3-Chloro-5-(7-chloro-1-propyl-1H-benzoimidazol-2-yl)-pyridin-2-yl]-(2-fluoro-phenyl)-amine

A mixture of 7-chloro-2-(5,6-dichloro-pyridin-3-yl)-1-propyl-1H-benzoimidazole (250 mg, 0.73 mmol) and 2-fluoroaniline (1.57 ml, 14.7 mmol) was heated to 150° C. for 3 h in a microwave oven. The mixture was then concentrated in vacuo and the crude product was purified by flash chromatography (Hex/EtOAc 100:0 to 70:30) and re-crystallization from Hex/EtOAc to provide the desired product (130 mg, 43%). UPLC (5-100% CH₃CN): t_{R}=1.549 min, TLC (Hex/EtOAc 4:1): R_{f}=0.34

The starting material is prepared as described below:

### 7-Chloro-2-(5,6-dichloro-pyridin-3-yl)-1-propyl-1H-benzoimidazole

A mixture of 5,6-dichloro-nicotinic acid (25 g, 128 mmol) and SOCl₂ (10.6 ml, 146 mmol) in toluene (125 ml) was treated with DMF (197 μl, 2.55 mmol) at RT and the mixture was then heated to 100° C. for 18 h. The mixture was allowed to cool to RT and then concentrated in vacuo to give the crude acid chloride, which was diluted in anhydrous THF (500 ml) and treated with 3-chloro-N-2-propyl-benzene-1,2-diamine (24.6 g, 133 mmol). The solution was stirred at RT for 2 h, and then heated to 60° C. for 4 h. the mixture was cooled to RT and stirred for 18 h. The precipitate was filtered and dried in vacuo. The crude product was taken up in EtOAc and washed with a sat. aq. soln. of NaHCO₃, dried over Na₂SO₄, filtered and concentrated in vacuo to give pure 7-chloro-2-(5,6-dichloro-pyridin-3-yl)-1-propyl-1H-benzoimidazole (30.3 g, 67%). HPLC (System 3, 30-100% CH₃CN): t_{R}=3.531 min, TLC (Hex/EtOAc 4:1): R_{f}=0.46.

By analogy to example 53, the following compounds can be prepared:

### EXAMPLE 54

### [3-Chloro-5-(7-chloro-1-propyl-1H-benzoimidazol-2-yl)-pyridin-2-yl]-(4-fluoro-phenyl)-amine; UPLC (5-100% CH₃CN): t_{R}=1.518 min; TLC (Hex/EtOAc 4:1): R_{f}=0.34

### EXAMPLE 55

### [3-Chloro-5-(7-chloro-1-propyl-1H-benzoimidazol-2-yl)-pyridin-2-yl]-(3-fluoro-phenyl)-amine; UPLC (5-100% CH₃CN): t_{R}=1.579 min; TLC (Hex/EtOAc 4:1): R_{f}=0.34

### EXAMPLE 56

### 4-[3-Chloro-5-(7-chloro-1-propyl-1H-benzoimidazol-2-yl)-pyridin-2-ylamino]-benzonitrile; UPLC (5-100% CH₃CN): t_{R}=1.511 min; TLC (Hex/EtOAc 2:1): R_{f}=0.25

### EXAMPLE 57

### [3-Chloro-5-(7-chloro-1-propyl-1H-benzoimidazol-2-yl)-pyridin-2-yl]-(3,4-difluoro-phenyl)-amine; UPLC (5-100% CH₃CN): t_{R}=1.594 min; TLC (Hex/EtOAc 4:1): R_{f}=0.25

### EXAMPLE 58

### [3-Chloro-5-(7-chloro-1-propyl-1H-benzoimidazol-2-yl)-pyridin-2-yl]-(2,3-difluoro-phenyl)-amine; UPLC (5-100% CH₃CN): t_{R}=1.581 min; TLC (Hex/EtOAc 4:1): R_{f}=0.38

### EXAMPLE 59

### [3-Chloro-5-(7-chloro-1-propyl-1H-benzoimidazol-2-yl)-pyridin-2-yl]-pyridin-4-yl-amine

A mixture of 7-chloro-2-(5,6-dichloro-pyridin-3-yl)-1-propyl-1H-benzoimidazole (800 mg, 2.35 mmol), 4-aminopyridine (271 mg, 2.82 mmol), Pd(OAc)₂ (11 mg, 0.05 mmol), rac-BINAP (29 mg, 0.05 mmol), and Cs₂CO₃ (3.9 g, 11.7 mmol) in toluene (20 ml) was heated to reflux for 18 h. It was then allowed to cool to RT and the mixture was filtered. The filtrate was concentrated in vacuo and the crude product was purified by flash chromatography (Hex/EtOAc 100:0 to 0:100)) and re-crystallization from Hex/EtOAc to afford the title compound (457 mg, 49%). HPLC (System 3, 30-100% CH₃CN): t_{R}=1.876 min, TLC (EtOAc): R_{f}=0.42.

The following compound can be prepared analogously:

### EXAMPLE 60

### [3-Chloro-5-(7-chloro-1-propyl-1H-benzoimidazol-2-yl)-pyridin-2-yl]-(6-methyl-pyridazin-3-yl)-amine; HPLC (System 2, 10-100% CH₃CN): t_{R}=3.063 min; TLC (EtOAc): R_{f}=0.43

### EXAMPLE 61

### [3-Chloro-5-(7-chloro-1-propyl-1H-benzoimidazol-2-yl)-pyridin-2-yl]-quinoxalin-6-yl-amine; HPLC (System 3, 30-100% CH₃CN): t_{R}=2.872 min; TLC (Hex/EtOAc 1:1): R_{f}=0.24

### EXAMPLE 62

### [3-Chloro-5-(7-chloro-1-propyl-1H-benzoimidazol-2-yl)-pyridin-2-yl]-(5-methyl-pyrazin-2-yl)-amine; HPLC (System 3, 30-100% CH₃CN): t_{R}=2.880 min; TLC (Hex/EtOAc 1:1): R_{f}=0.37

### EXAMPLE 63

### [3-Chloro-5-(7-chloro-1-propyl-1H-benzoimidazol-2-yl)-pyridin-2-yl]-quinolin-3-yl-amine; HPLC (System 3, 30-100% CH₃CN): t_{R}=2.624 min; TLC (Hex/EtOAc 1:1): R_{f}=0.37

### EXAMPLE 64

### [3-Chloro-5-(7-chloro-1-propyl-1H-benzoimidazol-2-yl)-pyridin-2-yl]-quinolin-6-yl-amine; HPLC (System 3, 30-100% CH₃CN): t_{R}=2.225 min; TLC (Hex/EtOAc 1:1): R_{f}=0.29

### EXAMPLE 65

### [3-Chloro-5-(6-fluoro-1-propyl-1H-benzoimidazol-2-yl)-pyridin-2-yl]-p-tolyl-amine; HPLC (System 3, 30-100% CH₃CN): t_{R}=2.908 min; TLC (Hex/EtOAc 3:1): R_{f}=0.36

### EXAMPLE 66

### [3-Chloro-5-(6-fluoro-1-propyl-1H-benzoimidazol-2-yl)-pyridin-2-yl]-(4-methoxy-phenyl)-amine; HPLC (System 3, 30-100% CH₃CN): t_{R}=2.610 min; TLC (Hex/EtOAc 3:1): R_{f}=0.25

### EXAMPLE 67

### [3-Chloro-5-(7-chloro-1-propyl-1H-benzoimidazol-2-yl)-pyridin-2-yl]-(2,6-difluoro-phenyl)-amine; UPLC (5-100% CH₃CN): t_{R}=1.452 min; TLC (Hex/EtOAc 4:1): R_{f}=0.19

### EXAMPLE 68

### N-5-[3-Chloro-5-(6-fluoro-1-propyl-1H-benzoimidazol-2-yl)-pyridin-2-yl]-N-2,N-2-dimethyl-pyridine-2,5-diamine; HPLC (System 2, 10-100% CH₃CN): t_{R}=2.625 min; TLC (EtOAc): R_{f}=0.63

The starting material is prepared as described below:

### N-2,N-2-Dimethyl-pyridine-2,5-diamine

A solution of 2-chloro-5-nitro-pyridine (10 g, 63.1 mmol) in EtOH (170 ml) was treated dropwise with dimethylamine (25% solution in H₂O 43 ml, 210 mmol). The mixture was heated to 80° C. for 1 h and then allowed to cool to RT. A precipitate formed and it was filtered, washed with cold EtOH, and dried. This solid was then diluted with THF, treated with Pd/C (10%, 655 mg) and stirred at RT under H₂ for 1 h. The mixture was then filtered and concentrated in vacuo to give N-2,N-2-dimethyl-pyridine-2,5-diamine as a red liquid (6.8 g, 94%). HPLC (System 1, 0-100% CH₃CN): t_{R}=0.858 min, TLC (EtOAc/NH₃ 99:1): R_{f}=0.38.

### EXAMPLE 69

### N-5-[3-Chloro-5-(1-propyl-1H-benzoimidazol-2-yl)-pyridin-2-yl]-N2,N-2-dimethyl-pyridine-2,5-diamine; HPLC (System 2, 10-100% CH₃CN): t_{R}=2.452 min; TLC (EtOAc): R_{f}=0.58

### EXAMPLE 70

### N-5-[3-Chloro-5-(7-chloro-1-propyl-1H-benzoimidazol-2-yl)-pyridin-2-yl]-N2,N-2-dimethyl-pyridine-2,5-diamine; HPLC (System 2, 10-100% CH₃CN): t_{R}=2.949 min; TLC (EtOAc): R_{f}=0.68

### EXAMPLE 71

### N-{5-[3-Chloro-5-(6-fluoro-1-propyl-1H-benzoimidazol-2-yl)-pyridin-2-ylamino]-pyridin-2-yl}-acetamide; HPLC (System 2, 10-100% CH₃CN): t_{R}=2.708 min; TLC (EtOAc): R_{f}=0.61

### EXAMPLE 71b

### N-5-[3-Chloro-5-(6-fluoro-1-propyl-1H-benzoimidazol-2-yl)-pyridin-2-yl]-pyridine-2,5-diamine

A solution of N-{5-[3-Chloro-5-(6-fluoro-1-propyl-1H-benzoimidazol-2-yl)-pyridin-2-ylamino]-pyridin-2-yl}-acetamide (350 mg, 0.78 mmol) in 4M aq. HCl (10 ml) was heated to reflux for 6 h. The mixture was allowed to cool to RT, and neutralized with saturated aq. NaHCO₃. The precipitate was filtered and re-crystallized in EtOAc to yield N-5-[3-Chloro-5-(6-fluoro-1-propyl-1H-benzoimidazol-2-yl)-pyridin-2-yl]-pyridine-2,5-diamine (150 mg, 47%). HPLC (System 2, 10-100% CH₃CN): t_{R}=2.559 min, TLC (EtOAc): R_{f}=0.39.

### EXAMPLE 72

### [3-Chloro-5-(7-chloro-1-propyl-1H-benzoimidazol-2-yl)-pyridin-2-yl]-pyrazin-2-yl-amine; HPLC (System 3, 30-100% CH₃CN): t_{R}=2.804 min; TLC (Hex/EtOAc 1:1): R_{f}=0.27

### EXAMPLE 73

### [3-Chloro-5-(7-chloro-1-propyl-1H-benzoimidazol-2-yl)-pyridin-2-yl]-(3-methyl-isoxazol-5-yl)-amine; HPLC (System 3, 30-100% CH₃CN): t_{R}=2.917 min; TLC (Hex/EtOAc 1:1): R_{f}=0.46

### EXAMPLE 74

### [3-Chloro-5-(7-chloro-1-propyl-1H-benzoimidazol-2-yl)-pyridin-2-yl]-(6-methoxy-pyridin-3-yl)-amine; HPLC (System 3, 30-100% CH₃CN): t_{R}=2.613 min; TLC (Hex/EtOAc 1:1): R_{f}=0.37

### EXAMPLE 75

### [3-Chloro-5-(7-chloro-1-propyl-1H-benzoimidazol-2-yl)-pyridin-2-yl]-(4-methoxy-phenyl)-amine; HPLC (System 3, 30-100% CH₃CN): t_{R}=3.130 min; TLC (Hex/EtOAc 1:1): R_{f}=0.63

### EXAMPLE 75b

### 5-[3-Chloro-5-(7-chloro-1-propyl-1H-benzoimidazol-2-yl)-pyridin-2-ylamino]-pyridine-2-carbonitrile; HPLC (System 3, 30-100% CH₃CN): t_{R}=3.109 min; TLC (Hex/EtOAc 1:1): R_{f}=0.48

### EXAMPLE 75c

### N-{5-[3-Chloro-5-(7-chloro-1-propyl-1H-benzoimidazol-2-yl)-pyridin-2-ylamino]-pyridin-2-yl}-acetamide; HPLC (System 3, 30-100% CH₃CN): t_{R}=2.054 min; TLC (EtOAc): R_{f}=0.55

### EXAMPLE 75d

### 5-[3-Chloro-5-(7-chloro-1-propyl-1H-benzoimidazol-2-yl)-pyridin-2-ylamino]-pyridine-2-carboxylic acid methyl ester; HPLC (System 3, 30-100% CH₃CN): t_{R}=2.751 min; TLC (EtOAc): R_{f}=0.66

### EXAMPLE 75e

### N-5-[3-Chloro-5-(7-chloro-1-propyl-1H-benzoimidazol-2-yl)-pyridin-2-yl]-pyridine-2,5-diamine

A solution of N-{5-[3-Chloro-5-(7-chloro-1-propyl-1H-benzoimidazol-2-yl)-pyridin-2-ylamino]-pyridin-2-yl}-acetamide (500 mg, 1.10 mmol) in 4M HCl (10 ml) was heated to 100° C. for 6 h. The mixture was allowed to cool to RT, neutralized with saturated aq. NaHCO₃, and extracted with EtOAc. The combined org. phases were dried over Na₂SO₄, filtered and concentrated in vacuo. Purification by re-crystallization from Hexan/EtoAc provided the desired product (335 mg, 74%). HPLC (System 3, 30-100% CH₃CN): t_{R}=1.554 min, TLC (EtOAc): R_{f}=0.43.

### EXAMPLE 75f

### {5-[3-Chloro-5-(7-chloro-1-propyl-1H-benzoimidazol-2-yl)-pyridin-2-ylamino]-pyridin-2-yl}-methanol

A solution of 5-[3-chloro-5-(7-chloro-1-propyl-1H-benzoimidazol-2-yl)-pyridin-2-ylamino]-pyridine-2-carboxylic acid methyl ester (1.5 g, 3.29 mmol) in anhydrous THF (25 ml) was cooled to 0° C. and treated with LiAlH₄ (193 mg, 4.93 mmol). The mixture was allowed to warm to RT and then stirred for 18 h. The mixture was cooled again to 0° C. and then treated dropwise with water (0.2 ml), aq. 1N NaOH solution (0.2 ml), and water (0.6 ml) again. The resulting suspension was diluted with EtOAc, filtered and the filtrate was concentrated in vacuo. Purification by flash chromatography (Hex/EtOAc 100:0 to 0:100) and re-crystallization from EtOAc afforded the desired product (515 mg, 37%). HPLC (System 2, 10-100% CH₃CN): t_{R}=2.900 min, TLC (EtOAc): R_{f}=0.50.

### EXAMPLE 76

### [6-(6-Fluoro-1-propyl-1H-benzoimidazol-2-yl)-pyridin-3-yl]-(6-methyl-pyridin-3-yl)-amine

A mixture of 5-(6-methyl-pyridin-3-ylamino)-pyridine-2-carboxylic acid (400 mg, 1.74 mmol) and 4-fluoro-N-2-propyl-benzene-1,2-diamine (352 mg, 2.09 mmol) in PPA (4 ml) was heated to 200° C. for 18 h. The mixture was poured onto ice/water and stirred for 2 h. The pH was adjusted to 8 with a 20% aq. soln. of NaOH and the mixture was extracted with EtOAc. The combined org layers were dried over Na₂SO₄, filtered and concentrated in vacuo. Purification by flash chromatography (DCM/MeOH 100:0 to 95:5) provided the desired product (45 mg, 7%). HPLC (System 2, 10-100% CH₃CN): t_{R}=2.550 min, MS (ES+): 362 [M+1].

The starting materials are prepared as described below:

### 5-(6-Methyl-pyridin-3-ylamino)-pyridine-2-carboxylic Acid

A solution of 5-(6-methyl-pyridin-3-ylamino)-pyridine-2-carboxylic acid methyl ester (2.3 g, 9.45 mmol) in MeOH (40 ml) was treated dropwise with a 2 N aq. soln of NaOH (9.5 ml, 19 mmol) and the mixture was stirred for 18 h at RT. The MeOH was evaporated in vacuo and the residue was taken up in water (25 ml) and extracted with EtOAc. The pH of the water phase was then set to 5-6 with conc. HCl and the mixture was concentrated in vacuo. The residue was taken up in EtOh and heated to reflux for 30 min. Upon cooling to RT, a precipitate formed, and was filtered and dried in vacuo to give 5-(6-methyl-pyridin-3-ylamino)-pyridine-2-carboxylic acid. MS (ES−): 228 [M−1].

### 5-(6-Methyl-pyridin-3-ylamino)-pyridine-2-carboxylic Acid Methyl Ester

A mixture of 5-bromo-pyridine-2-carboxylic acid methyl ester (2.5 g, 11.3 mmol), 6-methyl-pyridin-3-ylamine (1.84 g, 17.0 mmol), Pd(OAc)₂ (76 mg, 0.34 mmol), rac-BINAP (212 mg, 0.34 mmol), and K₂CO₃ (7.84 g, 56.7 mmol) in dioxane (75 ml) was heated to 90° C. for 18 h. The mixture was allowed to cool to RT and then concentrated in vacuo. Purification by flash chromatography (DCM/MeOH 100:0 to 95:5) afforded 5-(6-methyl-pyridin-3-ylamino)-pyridine-2-carboxylic acid methyl ester (2.38 g, 86%). HPLC (System 1, 0-100% CH₃CN): t_{R}=2.397 min, MS (ES+): 244 [M+1].

### EXAMPLE 77

### (4-Chloro-phenyl)-[4-(6-fluoro-1-propyl-1H-benzoimidazol-2-yl)-phenyl]-amine

A suspension of 2-(4-Bromo-phenyl)-6-fluoro-1-propyl-1H-benzoimidazole (400 mg, 1.20 mmol), 4-chloro-aniline (232 mg, 1.80 mmol), rac-BINAP (22 mg, 0.04 mmol), Pd(OAc)₂ (8 mg, 0.04 mmol) and K₂CO₃ (830 mg, 6.0 mmol) in dioxane (25 ml) was heated to 90° C. for 18 h. The mixture was allowed to cool to RT and was then filtered. The filtrate was taken up in EtOAc, and washed with water. The org. layer was then dried over Na₂SO₄, filtered and concentrated in vacuo. Purification by flash chromatography (Hex/EtOAc 100:0 to 50:50) and re-crystallization from Hex/EtOAc afforded the desired product (223 mg, 49%). HPLC (System 3, 30-100% CH₃CN): t_{R}=2.796 min, MS (ES+): 380 [M+1].

The starting material can be prepared as described below:

### 2-(4-Bromo-phenyl)-6-fluoro-1-propyl-1H-benzoimidazole

A mixture of 4-bromo-benzoic acid (1.5 g, 7.39 mmol) and 4-fluoro-N-2-propyl-benzene-1,2-diamine (1.49 g, 8.86 mmol) in PPA (10 ml) was heated to 200° C. for 18 h. The mixture was poured onto ice/water and stirred for 4 h. The pH was adjusted to 8 with a 20% aq. soln. of NaOH and the mixture was extracted with EtOAc. The combined org layers were dried over Na₂SO₄, filtered and concentrated in vacuo. Purification by flash chromatography (Hex/EtOAc 100:0 to 80:20) provided the desired product (1.43 g, 58%). HPLC (System 3, 30-100% CH₃CN): t_{R}=2.104 min, MS (ES+): 334 [M+1].

### EXAMPLE 78

### [4-(7-Chloro-1-propyl-1H-benzoimidazol-2-yl)-phenyl]-(6-methyl-pyridin-3-yl)-amine

A mixture of 4-(6-Methyl-pyridin-3-ylamino)-benzoic acid (600 mg, 2.63 mmol) and 3-chloro-N-2-propyl-benzene-1,2-diamine (583 mg, 3.16 mmol) in PPA (3 ml) was heated to 200° C. for 15 min in a microwave oven. The mixture was poured onto ice/water and stirred for 30 min. The pH was adjusted to 8 with a 20% aq. soln. of NaOH and the mixture was extracted with EtOAc. The combined org layers were dried over Na₂SO₄, filtered and concentrated in vacuo. Purification by flash chromatography (EtOAc) and re-crystallization from Hex/EtOAc provided the desired product (221 mg, 22%). HPLC (System 2, 10-100% CH₃CN): t_{R}=2.680 min, MS (ES+): 377 [M+1].

The starting materials are prepared as described below:

### 4-(6-Methyl-pyridin-3-ylamino)-benzoic Acid

A solution of 4-(6-methyl-pyridin-3-ylamino)-benzoic acid ethyl ester (2.2 g, 8.58 mmol) in MeOH (50 ml) was treated with a 2 N aq. solution of NaOH (8.6 ml, 17.0 mmol) and the mixture was heated to 50° C. for 18 h. the mixture was then neutralized with a 2 M solution of HCl and the mixture was cooled to 0° C. with an ice/water bath. The precipitate was filtered and dried in vacuo to give 4-(6-methyl-pyridin-3-ylamino)-benzoic acid (1.61 g, 82%). HPLC (System 1, 0-100% CH₃CN): t_{R}=2.421 min, MS (ES+): 229 [M+1].

### 4-(6-Methyl-pyridin-3-ylamino)-benzoic Acid Ethyl Ester

A suspension of 4-iodo-benzoic acid ethyl ester (2.5 g, 9.06 mmol), 3-amino-6-methylpyridine (1.18 g, 10.9 mmol), rac-BINAP (113 mg, 0.18 mmol), Pd(OAc)₂ (41 mg, 0.18 mmol) and Cs₂CO₃ (15.1 g, 45.4 mmol) in toluene (90 ml) was heated to reflux for 5 h. The mixture was cooled to RT, filtered and concentrated in vacuo. Purification by flash chromatography (Hex/EtOAc 50:50) provided 4-(6-methyl-pyridin-3-ylamino)-benzoic acid ethyl ester (2.27 g, 98%).

HPLC (System 2, 10-100% CH₃CN): t_{R}=2.537 min, MS (ES+): 257 [M+1].

By analogy to example 78, the following compound can be prepared:

### EXAMPLE 79

### [4-(6-Fluoro-1-propyl-1H-benzoimidazol-2-yl)-phenyl]-(6-methyl-pyridin-3-yl)-amine; HPLC (System 2, 10-100% CH₃CN): t_{R}=2.435 min; TLC (EtOAc): R_{f}=0.42

### EXAMPLE 80

### [4-(7-Chloro-1-propyl-1H-benzoimidazol-2-yl)-phenyl]-p-tolyl-amine

A suspension of 4-chloro-2-(4-iodo-phenyl)-3-propyl-3H-benzoimidazolium hydrochloride (5.0 g, 11.5 mmol), para-toluidine (1.51 g, 13.8 mmol), rac-BINAP (144 mg, 0.23 mmol), Pd(OAc)₂ (52 mg, 0.23 mmol) and Cs₂CO₃ (19.2 g, 57.7 mmol) in toluene (20 ml) was heated to reflux for 18 h. The mixture was cooled to RT, filtered and concentrated in vacuo. Purification by flash chromatography (Hex/EtOAc 75:25) and re-crystallization from Hex/EtOAc gave [4-(7-chloro-1-propyl-1H-benzoimidazol-2-yl)-phenyl]-p-tolyl-amine (2.17 g, 50%). HPLC (System 3, 30-100% CH₃CN): t_{R}=3.072 min, TLC (Hex/EtOAc 3:1): R_{f}=0.24.

The starting material can be prepared as described below:

### 4-Chloro-2-(4-iodo-phenyl)-3-propyl-3H-benzoimidazolium Hydrochloride

A solution of 4-iodo-benzoyl chloride (10.0 g, 37.5 mmol) and 3-chloro-N-2-propyl-benzene-1,2-diamine (7.14 g, 38.7 mmol) in anhydrous THF (200 ml) was heated to 60° C. for 1 h. The mixture was allowed to cool to RT and stirred for another 18 h. The mixture was cooled to 0° C. with an ice/water bath and the precipitate was filtered and concentrated in vacuo to give 4-chloro-2-(4-iodo-phenyl)-3-propyl-3H-benzoimidazolium hydrochloride (11.56 g, 71%). HPLC (System 3, 30-100% CH₃CN): t_{R}=3.073 min, TLC (Hex/EtOAc 4:1): R_{f}=0.53.

The following compounds can be prepared by analogy:

### EXAMPLE 81

### (4-Chloro-phenyl)-[4-(7-chloro-1-propyl-1H-benzoimidazol-2-yl)-phenyl]-amine; HPLC (System 3, 30-100% CH₃CN): t_{R}=3.071 min; TLC (Hex/EtOAc 1:1): R_{f}=0.19

### EXAMPLE 82

### [4-(7-Chloro-1-propyl-1H-benzoimidazol-2-yl)-phenyl]-(4-methoxy-phenyl)-amine; HPLC (System 3, 30-100% CH₃CN): t_{R}=2.889 min; TLC (Hex/EtOAc 1:1): R_{f}=0.23

### EXAMPLE 83

### [4-(6-Fluoro-1-propyl-1H-benzoimidazol-2-yl)-phenyl]-p-tolyl-amine; HPLC (System 3, 30-100% CH₃CN): t_{R}=2.859 min; TLC (Hex/EtOAc 2:1): R_{f}=0.35

### EXAMPLE 84

### (4-Chloro-phenyl)-[6-(1-propyl-1H-benzoimidazol-2-yl)-pyridazin-3-yl]-amine

A mixture of 6-(4-chloro-phenylamino)-pyridazine-3-carboxylic acid (50% pure, 400 mg, 0.80 mmol) and N-propyl-benzene-1,2-diamine (144 mg, 0.96 mmol) in PPA (3 ml) was heated to 200° C. for 1 h. The mixture was then poured onto ice/water. The pH was adjusted to 8 with a 20% aq. soln. of NaOH and the mixture was extracted with EtOAc. The combined org layers were dried over Na₂SO₄, filtered and concentrated in vacuo. Purification by flash chromatography (Hex/EtOAc 100:0 to 0:100) and re-crystallization from Hex/EtOAc provided the desired product (72 mg, 25%). UPLC (5-100% CH₃CN): t_{R}=1.342 min, MS (ES−): 362 [M−1].

During the purification by flash chromatography, a side-product can be isolated:

6-(1-Propyl-1H-benzoimidazol-2-yl)-pyridazin-3-ol; UPLC (5-100% CH₃CN): t_{R}=0.835 min; MS (ES+): 255 [M+1]

The starting materials can be prepared as described below:

### 6-(4-Chloro-phenylamino)-pyridazine-3-carboxylic Acid

A mixture of 6-chloro-pyridazine-3-carboxylic acid (80% pure, 400 mg, 2.02 mmol) and 4-chloro-aniline (523 mg, 4.06 mmol) in 1,2-DME (10 ml) was heated to 80° C. for 90 min in the microwave oven. The mixture was allowed to cool to RT and then concentrated in vacuo. Purification by flash chromatography (Hex/EtOAc 100:0 to 0:100) gave 6-(4-chloro-phenylamino)-pyridazine-3-carboxylic acid (50% pure, 330 mg, 33%). UPLC (5-100% CH₃CN): t_{R}=0.915 min, MS (ES−): 248 [M−1].

### 6-Chloro-pyridazine-3-carboxylic Acid

A mixture of 3-chloro-6-methyl-pyridazine (10.0 g, 77.8 mmol) and K₂Cr₂O₇ (38.1 g, 128 mmol) in conc. H₂SO₄ (150 ml) was heated to 60° C. for 24 h. The mixture was poured onto ice/water and extracted with EtOAc. The combined org. layers were then concentrated in vacuo and then taken up in EtOAc. The suspension was filtered and dried in vacuo to give 6-chloro-pyridazine-3-carboxylic acid (80% pure, 4.1 g, 27%). UPLC (5-100% CH₃CN): t_{R}=0.523 min, MS (ES−): 157 [M−1].

### EXAMPLE 85

### (6-Methyl-pyridin-3-yl)-[6-(1-propyl-1H-benzoimidazol-2-yl)-pyridazin-3-yl]-amine

A mixture of 2-(6-chloro-pyridazin-3-yl)-1-propyl-1H-benzoimidazole (110 mg, 0.40 mmol), 3-amino-6-methylpyridine (174 mg, 1.61 mmol), rac-BINAP (15 mg, 0.02 mmol), Pd(OAc)₂ (5 mg, 0.02 mmol) and K₂CO₃ (168 mg, 1.22 mmol) in toluene (50 ml) was heated to 100° C. for 30 min. The mixture was then concentrated in vacuo and purified by flash chromatography (DCM/MeOH 100:0 to 85:15) and re-crystallization from Hex/EtOAc to provide the desired product (20 mg, 14%). UPLC (5-100% CH₃CN): t_{R}=0.816 min, MS (ES+): 345 [M+1].

### 2-(6-Chloro-pyridazin-3-yl)-1-propyl-1H-benzoimidazole

A solution of 6-(1-propyl-1H-benzoimidazol-2-yl)-pyridazin-3-ol (from example 62, 240 mg, 0.94 mmol) in POCl₃ (20 ml) was heated to 80° C. for 5 h. The mixture was then diluted with water and extracted with EtOAc. The combined org. phases were dried over Na₂SO₄, filtered, and concentrated in vacuo to give 2-(6-chloro-pyridazin-3-yl)-1-propyl-1H-benzoimidazole (120 mg, 47%). UPLC (5-100% CH₃CN): t_{R}=1.233 min, MS (ES+): 273 [M+1].

### EXAMPLE 86

### [6-(7-Chloro-1-propyl-1H-benzoimidazol-2-yl)-pyridazin-3-yl]-(4-methoxy-phenyl)-amine

A mixture of 7-chloro-2-(6-chloro-pyridazin-3-yl)-1-propyl-1H-benzoimidazole (500 mg, 1.63 mmol) and para-anisidine (411 mg, 3.27 mmol) in 1,2-DME (10 ml) was heated to 130° C. for 30 min in the microwave oven. The mixture was allowed to cool to RT and then concentrated in vacuo. Purification by flash chromatography (Hex/EtOAc 100:0 to 60:40) and re-crystallization from Hex/DCM afforded [6-(7-chloro-1-propyl-1H-benzoimidazol-2-yl)-pyridazin-3-yl]-(4-methoxy-phenyl)-amine (65 mg, 10%). UPLC (5-100% CH₃CN): t_{R}=1.395 min, TLC (Hex/EtOAc 1:1): R_{f}=0.25.

The starting materials can be prepared as described below:

### 7-Chloro-2-(6-chloro-pyridazin-3-yl)-1-propyl-1H-benzoimidazole

A solution of 6-(7-chloro-1-propyl-1H-benzoimidazol-2-yl)-pyridazin-3-ol (4.5 g, 15.6 mmol) in POCl3 (50 ml) was heated to 80° C. for 1 h. The excess of POCl₃ was evaporated in vacuo and the residue was taken up in toluene and concentrated again in vacuo to give 7-chloro-2-(6-chloro-pyridazin-3-yl)-1-propyl-1H-benzoimidazole (4.7 g, 98%), which was used without further purification. UPLC (5-100% CH₃CN): t_{R}=1.697 min, MS (ES+): 307 [M+1].

### 6-(7-Chloro-1-propyl-1H-benzoimidazol-2-yl)-pyridazin-3-ol

A mixture of 6-chloro-pyridazine-3-carboxylic acid (8.6 g, 54.2 mmol) and 3-chloro-N-2-propyl-benzene-1,2-diamine (9.62 g, 52.1 mmol) in PPA (50 ml) was heated to 200° C. for 12 h. The mixture was then poured onto ice/water. The pH was adjusted to 8 with a 20% aq. soln. of NaOH and the mixture was extracted with EtOAc. The combined org layers were dried over Na₂SO₄, filtered and concentrated in vacuo. Purification by flash chromatography (Hex/EtOAc 100:0 to 0:100) furnished 6-(7-chloro-1-propyl-1H-benzoimidazol-2-yl)-pyridazin-3-ol (4.5 g, 29%). UPLC (5-100% CH₃CN): t_{R}=1.235 min, MS (ES+): 289 [M+1].

The following compound can be prepared by analogy:

### EXAMPLE 87

### (4-Chloro-phenyl)-[6-(7-chloro-1-propyl-1H-benzoimidazol-2-yl)-pyridazin-3-yl]-amine; UPLC (5-100% CH₃CN): t_{R}=1.696 min; TLC (Hex/EtOAc 1:1): R_{f}=0.25

### EXAMPLE 88

### [6-(7-Chloro-1-propyl-1H-benzoimidazol-2-yl)-pyridazin-3-yl]-(6-methyl-pyridin-3-yl)-amine

A mixture of 7-chloro-2-(6-chloro-pyridazin-3-yl)-1-propyl-1H-benzoimidazole (3.7 g, 12.0 mmol), 3-amino-6-methylpyridine (5.2 g, 48.1 mmol), rac-BINAP (448 mg, 0.72 mmol), Pd(OAc)₂ (135 mg, 0.60 mmol) and K₂CO₃ (5.01 g, 36.2 mmol) in toluene (50 ml) was heated to 120° C. for 20 h. The mixture was then concentrated in vacuo and purified by flash chromatography (Hex/EtOAc/MeOH 100:0:0 to 0:80:20) to provide the desired product (240 mg, 5%). UPLC (5-100% CH₃CN): t_{R}=1.131 min, TLC (DCM/MeOH 9:1): R_{f}=0.30.

### EXAMPLE 89

### (6-Methyl-pyridin-3-yl)-[5-(1-propyl-1H-benzoimidazol-2-yl)-pyrimidin-2-yl]-amine

A mixture of 2-(2-chloro-pyrimidin-5-yl)-1-propyl-1H-benzoimidazole (60 mg, 0.22 mmol), 3-amino-6-methylpyridine (95 mg, 0.88 mmol), rac-BINAP (8 mg, 0.01 mmol), Pd(OAc)₂ (3 mg, 0.01 mmol) and K₂CO₃ (92 mg, 0.66 mmol) in toluene (50 ml) was heated to 100° C. for 5 h. The mixture was then concentrated in vacuo and purified by flash chromatography (DCM/MeOH 100:0 to 90:10) and preparative TLC (DCM/MeOH 9:1) to provide the desired product (14 mg, 18%). UPLC (5-100% CH₃CN): t_{R}=0.736 min, MS (ES+): 345 [M+1].

### 2-(2-Chloro-pyrimidin-5-yl)-1-propyl-1H-benzoimidazole

A suspension of 2-hydroxy-pyrimidine-5-carboxylic acid (300 mg, 2.14 mmol) [J. Arukwe, K. Undheim, Acta. Chem. Scand. (1986) B40, 764-767], in POCl₃ (20 ml) was heated to 80° C. for 16 h. The excess of POCl₃ was evaporated and the residue was dried in HV for 2 h. The crude product was then taken up in anhydrous THF (30 ml), treated with N-propyl-benzene-1,2-diamine (354 mg, 2.36 mmol) and the solution was stirred for 1 h at RT. The mixture was then concentrated in vacuo. Purification by flash chromatography (100:0 to 75:25) afforded 2-(2-chloro-pyrimidin-5-yl)-1-propyl-1H-benzoimidazole (63 mg, 11%). UPLC (5-100% CH₃CN): t_{R}=0.974 min, MS (ES+): 273 [M+1].

### EXAMPLE 90

### [5-(7-Chloro-1-propyl-1H-benzoimidazol-2-yl)-pyrimidin-2-yl]-(4-methoxy-phenyl)-amine

A mixture of 7-chloro-2-(2-chloro-pyrimidin-5-yl)-1-propyl-1H-benzoimidazole (200 mg, 0.26 mmol) and para-anisidine (164 mg, 1.30 mmol) in acetic acid (80%, 2 ml) was heated to 130° C. for 1 h in a microwave oven. The mixture was then concentrated in vacuo. Purification by flash chromatography (Hex/EtOAc 100:0 to 0:100) and re-crystallization from Hex/DCM gave the desired product (40 mg, 39%). UPLC (5-100% CH₃CN): t_{R}=1.322 min, TLC (Hex/EtOAc 1:1): R_{f}=0.33.

The starting materials can be prepared as described below:

### 7-Chloro-2-(2-chloro-pyrimidin-5-yl)-1-propyl-1H-benzoimidazole

A suspension of 2-hydroxy-pyrimidine-5-carboxylic acid (3.25 g, 23.2 mmol) [J. Arukwe, K. Undheim (1986) Acta. Chem. Scand., B40, 764-767], in POCl₃ (100 ml) was heated to 80° C. for 48 h. The excess of POCl₃ was evaporated and the residue was dried in HV for 2 h. The crude product was then taken up in anhydrous THF (30 ml), treated with 3-chloro-N-2-propyl-benzene-1,2-diamine (4.35 g, 23.6 mmol) and the solution was stirred for 20 h at RT. The mixture was then concentrated in vacuo. Purification by flash chromatography (DCM/MeOH 100:0 to 85:15) provided 7-chloro-2-(2-chloro-pyrimidin-5-yl)-1-propyl-1H-benzoimidazole (40% pure, 8.0 g, 46%). UPLC (5-100% CH₃CN): t_{R}=1.462 min, MS (ES+): 307 [M+1].

The following compound can be prepared by analogy:

### EXAMPLE 91

### (4-Chloro-phenyl)-[5-(7-chloro-1-propyl-1H-benzoimidazol-2-yl)-pyrimidin-2-yl]-amine; UPLC (5-100% CH₃CN): t_{R}=1.510 min; TLC (Hex/EtOAc 1:1): R_{f}=0.39.

### EXAMPLE 92

### [5-(7-Chloro-1-propyl-1H-benzoimidazol-2-yl)-pyrimidin-2-yl]-(6-methyl-pyridin-3-yl)-amine

A mixture of 7-chloro-2-(2-chloro-pyrimidin-5-yl)-1-propyl-1H-benzoimidazole (200 mg, 0.65 mmol), 3-amino-6-methylpyridine (281 mg, 2.60 mmol), rac-BINAP (24 mg, 0.04 mmol), Pd(OAc)₂ (7 mg, 0.04 mmol) and K₂CO₃ (270 mg, 1.95 mmol) in toluene (50 ml) was heated to 120° C. for 48 h. The mixture was then concentrated in vacuo and purified by flash chromatography (Hex/EtOAc 100:0 to 0:100) to provide the desired product (25 mg, 10%). UPLC (5-100% CH₃CN): t_{R}=0.997 min, TLC (EtOAc): R_{f}=0.12.

### EXAMPLE 93

### [5-(6-Fluoro-1-propyl-1H-benzoimidazol-2-yl)-pyrimidin-2-yl]-(4-methoxy-phenyl)-amine

A suspension of 2-(4-methoxy-phenylamino)-pyrimidine-5-carboxylic acid (800 mg, 3.26 mmol) in SOCl₂ (20 ml) was heated to 80° C. for 15 min and then allowed to cool to RT. The mixture was concentrated in vacuo and subsequently diluted in anhydrous THF (40 ml). The solution was treated with 4-fluoro-N-2-propyl-benzene-1,2-diamine (606 mg, 3.60 mmol) and the mixture was heated to 70° C. for 16 h. The mixture was then concentrated in vacuo and diluted with an aq. solution of 2N NaOH. The aquous phase was extracted with EtOAc. The combined org. phases were then dried over Na₂SO₄, filtered and concentrated in vacuo. Purification by flash chromatography (Hex/EtOAc 100/0 to 30/70) provided the title compound (100 mg, 8%). UPLC (5-100% CH₃CN): t_{R}=1.185 min, TLC (Hex/EtOAc 1:1): R_{f}=0.31.

The starting material is prepared as described below:

### 2-(4-Methoxy-phenylamino)-pyrimidine-5-carboxylic Acid

A mixture of 2-methanesulfonyl-pyrimidine-5-carboxylic acid (5.5 g, 27.2 mmol) and p-anisidine (17.1 g, 136 mmol) was heated to 150° C. for 5 min. The mixture was then taken up in EtOAc, stirred for 10 min and the solid was filtered, taken up in MeOH and stirred for 30 min. Filtration gave 2-(4-methoxy-phenylamino)-pyrimidine-5-carboxylic acid (3.8 g, 54%). UPLC (5-100% CH₃CN): t_{R}=0.971 min, MS (ES+): 246 [M+1].

### 2-Methanesulfonyl-pyrimidine-5-carboxylic Acid

A suspension of 2-methylsulfanyl-pyrimidine-5-carboxylic acid (5.5 g, 32.3 mmol) [J. Arukwe, K. Undheim (1986) Acta. Chem. Scand., B40, 764-767] in DCM (500 ml) was treated portionwise with mCPBA (70%, 19.9 g, 81.0 mmol) and the mixture was stirred for 24 h at RT. The mixture was then filtered and the filtrated was concentrated in vacuo to give 2-methanesulfonyl-pyrimidine-5-carboxylic acid (5.1 g, 79%). UPLC (5-100% CH₃CN): t_{R}=0.451 min, MS (ES+): 203 [M+1].

### EXAMPLE 93b

### [5-(7-Chloro-5-iodo-1-propyl-1H-benzoimidazol-2-yl)-pyrimidin-2-yl]-(6-methyl-pyridin-3-yl)-amine

A suspension of 2-(6-methyl-pyridin-3-ylamino)-pyrimidine-5-carboxylic acid (360 mg, 1.56 mmol) in POCl₃ (10 ml) was heated to 100° C. for 4 h, and then concentrated in vacuo to give the acid chloride which was then diluted with anhydrous THF (20 ml) and treated with 3-chloro-5-iodo-N-2-propyl-benzene-1,2-diamine (581 mg, 1.87 mmol). The mixture was stirred for 24 h at RT, then diluted with a sat. aq. Na₂CO₃ solution, and extracted twice with EtOAc. The combined org phases were dried over Na₂SO₄, filtered and concentrated in vacuo. Purification by flash chromatography (Hex/EtOAc 100:0 to 0:100) and re-crystallization from EtOAc afforded the title compound (395 mg, 50%). UPLC (5-100% CH₃CN): t_{R}=1.331 min, TLC (EtOAc): R_{f}=0.30.

The starting material is prepared as described below:

### 2-(6-Methyl-pyridin-3-ylamino)-pyrimidine-5-carboxylic Acid

A solution of 2-(6-methyl-pyridin-3-ylamino)-pyrimidine-5-carboxylic acid ethyl ester (450 mg, 1.74 mmol) and KOH (171 mg, 2.62 mmol) in EtOH (20 ml) and water (5 ml) was stirred at RT for 2 h. The org. solvent was distilled off, and the mixture was rendered slightly acidic (pH 6) with an aq. 1N HCl solution. This mixture was stirred for 1 h at RT; the resulting suspension was filtered and the cake was dried in vacuo to provide 2-(6-methyl-pyridin-3-ylamino)-pyrimidine-5-carboxylic acid (365 mg, 91%). UPLC (5-100% CH₃CN): t_{R}=0.536 min, MS (ES+): 231 [M+1].

### 2-(6-Methyl-pyridin-3-ylamino)-pyrimidine-5-carboxylic Acid Ethyl Ester

A solution of 2-chloro-pyrimidine-5-carboxylic acid ethyl ester (9.40 g, 50.4 mmol) [K. Ohta, et al., Chem. Pharm. Bull. (2000), 48, 10, 1504-1513] and 6-methyl-pyridin-3-ylamine (5.99 g, 55.4 mmol) in DMSO (20 ml) was heated to 100° C. for 1 h. The mixture was poured onto water, diluted with a sat. aq. Na₂CO₃ solution, and extracted twice with EtOAc. The combined org. phases were then dried over Na₂SO₄, filtered and concentrated in vacuo. Purification by flash chromatography (Hex/EtOAc 100:0 to 0:100) and re-crystallization from EtOAc furnished 2-(6-methyl-pyridin-3-ylamino)-pyrimidine-5-carboxylic acid ethyl ester (4.8 g, 37%). UPLC (5-100% CH₃CN): t_{R}=0.777 min, TLC (EtOAc): R_{f}=0.50.

### EXAMPLE 94

### [2-Chloro-4-(1-propyl-1H-benzoimidazol-2-yl)-phenyl]-(6-methyl-pyridin-3-yl)-amine

A suspension of 2-(4-Bromo-3-chloro-phenyl)-1-propyl-1H-benzoimidazole (5 g, 14.3 mmol), 3-amino-6-methylpyridine (1.62 g, 15.0 mmol), rac-BINAP (890 mg, 1.43 mmol), Pd(OAc)₂ (321 mg, 1.43 mmol) and K₂CO₃ (9.88 g, 71.5 mmol) in toluene (200 ml) was heated to 120° C. for 24 h. The mixture was allowed to cool to RT diluted with EtOAc and washed twice with a aqueous solution of NaHCO₃ and water. The aqueous layer was extracted with EtOAc, and combined organic layers then dried over Na₂SO₄, filtered and concentrated in vacuo. Purification by flash chromatography (Hex/EtOAc 100:0 to 0:100) and re-crystallization from Et₂O afforded the desired product (2.75 g, 51%). HPLC (System 2, 10-100% CH₃CN): t_{R}=2.451 min, MS (LC-MS): 377 [M+1].

The starting material can be prepared as described below:

### 2-(4-Bromo-3-chloro-phenyl)-1-propyl-1H-benzoimidazole

A mixture of 4-bromo-3-chloro-benzoic acid (10 g, 41.2 mmol) and N-propyl-benzene-1,2-diamine (7.43 g, 49.5 mmol) in PPA (100 ml) was heated to 150° C. for 18 h. The mixture was poured onto ice/water. The pH was adjusted to 10 with a 30% aq. soln. of NaOH, filtered and extracted twice with EtOAc. The combined org layers were dried over Na₂SO₄, filtered and concentrated in vacuo. Purification by flash chromatography (Hex/EtOAc 100:0 to 30:70) provided the desired product (10.17 g, 71%). HPLC (System 3, 30-100% CH₃CN): t_{R}=2.263 min, MS (LC-MS): 350 [M+1].

### EXAMPLE 95

### [2-Chloro-4-(7-chloro-1-propyl-1H-benzoimidazol-2-yl)-phenyl]-p-tolyl-amine

A suspension of 2-(4-Bromo-3-chloro-phenyl)-7-chloro-1-propyl-1H-benzoimidazole (5 g, 13.0 mmol), p-toluidine (1.46 g, 13.6 mmol), rac-BINAP (809 mg, 1.30 mmol), Pd(OAc)₂ (292 mg, 1.30 mmol) and K₂CO₃ (9.98 g, 65 mmol) in toluene (200 ml) was heated to 120° C. for 18 h. The mixture was allowed to cool to RT diluted with EtOAc and washed twice with a aqueous solution of NaHCO₃ and water. The aqueous layer was extracted with EtOAc, and combined organic layers then dried over Na₂SO₄, filtered and concentrated in vacuo. Purification by flash chromatography (Hex/EtOAc 100:0 to 20:80) and re-crystallization from Et₂O/hexanes afforded the desired product (2.29 g, 43%). HPLC (System 3, 30-100% CH₃CN): t_{R}=3.379 min, MS (LC-MS): 411 [M+1].

The starting material can be prepared as described below:

### 2-(4-Bromo-3-chloro-phenyl)-7-chloro-1-propyl-1H-benzoimidazole

A mixture of 4-bromo-3-chloro-benzoic acid (10 g, 41.2 mmol) and 3-Chloro-2N-propyl-benzene-1,2-diamine (9.13 g, 49.4 mmol) in PPA (100 ml) was heated to 150° C. for 18 h. The mixture was poured onto ice/water. The pH was adjusted to 10 with a 30% aq. soln. of NaOH, filtered and extracted twice with EtOAc. The combined org layers were dried over Na₂SO₄, filtered and concentrated in vacuo. Purification by flash chromatography (Hex/EtOAc 100:0 to 20:80) provided the desired product (11.1 g, 70%). HPLC (System 3, 30-100% CH₃CN): t_{R}=3.591 min, MS (LC-MS): 358 [M+1].

Following compounds can be prepared by analogy:

### EXAMPLE 96

### [2-Chloro-4-(7-chloro-1-propyl-1H-benzoimidazol-2-yl)-phenyl]-(4-chloro-phenyl)-amine; HPLC (System 3, 30-100% CH₃CN): t_{R}=3.392 min; TLC (Hex/EtOAc 2:1): R_{f}=0.6.

### EXAMPLE 97

### [2-Chloro-4-(7-chloro-1-propyl-1H-benzoimidazol-2-yl)-phenyl]-(4-methoxy-phenyl)-amine; HPLC (System 3, 30-100% CH₃CN): t_{R}=3.165 min; TLC (Hex/EtOAc 2:1): R_{f}=0.48.

### EXAMPLE 98

### [2-Chloro-4-(7-chloro-1-propyl-1H-benzoimidazol-2-yl)-phenyl]-(6-methyl-pyridin-3-yl)-amine; HPLC (System 2, 10-100% CH₃CN): t_{R}=2.879 min; TLC (EtOAc): R_{f}=0.43.

### EXAMPLE 99

### [5-(7-Chloro-1-propyl-1H-benzoimidazol-2-yl)-pyridin-2-yl]-(4-methoxy-phenyl)-amine

A suspension of 7-Chloro-2-(6-chloro-pyridin-3-yl)-1-propyl-1H-benzoimidazole (0.15 g, 0.49 mmol), p-anisidine (0.091 g, 0.73 mmol), rac-BINAP (9.73 mg, 0.016 mmol), Pd(OAc)₂ (3.51 mg, 0.016 mmol) and K₂CO₃ (339 mg, 2.45 mmol) in toluene (3 ml) was heated to 100° C. for 17 h. The mixture was allowed to cool to RT diluted with DCM and washed with a aqueous solution of 1N HCl. The pH of the aqueous layer was adjusted to pH 10 and extracted with DCM. Combined organic layers were dried over Na₂SO₄, filtered and concentrated in vacuo. Re-crystallization from EtOAc afforded the desired product (70.7 mg, 37%). HPLC (System 3, 30-100% CH₃CN): t_{R}=2.499 min, MS (LC-MS): 393 [M+1].

The starting material can be prepared as described below:

### 7-Chloro-2-(6-chloro-pyridin-3-yl)-1-propyl-1H-benzoimidazole

A mixture of 6-Chloro-nicotinoyl chloride (500 mg, 2.84 mmol) and 3-Chloro-2N-propyl-benzene-1,2-diamine (630 mg, 3.41 mmol) in THF (15 ml) was stirred at 60° C. for 1.5 h. The mixture was concentrated in vacuo and purified by flash chromatography (Hex/EtOAc 100:0 to 50:50) to afford the desired product (318 mg, 37%). HPLC (System 3, 30-100% CH₃CN): t_{R}=2.860 min, MS (LC-MS): 307 [M+1].

Following compounds can be prepared by analogy:

### EXAMPLE 100

### [5-(7-Chloro-1-propyl-1H-benzoimidazol-2-yl)-pyridin-2-yl]-(6-methyl-pyridin-3-yl)-amine; HPLC (System 1, 0-100% CH₃CN): t_{R}=3.001 min; TLC (EtOAc): R_{f}=0.315.

### EXAMPLE 101

### (4-Chloro-phenyl)-[5-(7-chloro-1-propyl-1H-benzoimidazol-2-yl)-pyridin-2-yl]-amine; HPLC (System 3, 30-100% CH₃CN): t_{R}=3.035 min; TLC (EtOAc): R_{f}=0.481.

### EXAMPLE 102

### (6-Methyl-pyridin-3-yl)-[5-(1-propyl-1H-benzoimidazol-2-yl)-pyridin-2-yl]-amine; HPLC (System 2, 10-100% CH₃CN): t_{R}=2.322 min; MS (LC-MS): 344 [M+1].

The starting material can be prepared in analogy as described in example 99:

2-(6-Chloro-pyridin-3-yl)-1-propyl-1H-benzoimidazole; HPLC (System 2, 10-100% CH₃CN): t_{R}=2.635 min; MS (LC-MS): 272 [M+1].

### EXAMPLE 103

### [5-(7-Chloro-1-propyl-1H-benzoimidazol-2-yl)-pyridin-2-yl]-p-tolyl-amine; HPLC (System 3, 30-100% CH₃CN): t_{R}=2.813 min; MS (LC-MS): 377 [M+1].

### EXAMPLE 104

### (4-Chloro-phenyl)-[5-(6-fluoro-1-propyl-1H-benzoimidazol-2-yl)-pyridin-2-yl]-amine; HPLC (System 3, 30-100% CH₃CN): t_{R}=2.684 min; MS (LC-MS): 381 [M+1].

### EXAMPLE 105

### [5-(6-Fluoro-1-propyl-1H-benzoimidazol-2-yl)-pyridin-2-yl]-(6-methyl-pyridin-3-yl)-amine; HPLC (System 2, 10-100% CH₃CN): t_{R}=2.404 min; MS (LC-MS): 362 [M+1].

### EXAMPLE 106

### [5-(6-Fluoro-1-propyl-1H-benzoimidazol-2-yl)-pyridin-2-yl]-p-tolyl-amine; HPLC (System 3, 30-100% CH₃CN): t_{R}=2.451 min; MS (LC-MS): 361 [M+1].

### EXAMPLE 107

### [5-(6-Fluoro-1-propyl-1H-benzoimidazol-2-yl)-pyridin-2-yl]-(4-methoxy-phenyl)-amine; HPLC (System 3, 30-100% CH₃CN): t_{R}=2.069 min; MS (LC-MS): 377 [M+1].

### EXAMPLE 107b

### 5-[5-(7-Chloro-1-propyl-1H-benzoimidazol-2-yl)-pyridin-2-ylamino]-pyridine-2-carboxylic acid methyl ester; UPLC (5-100% CH₃CN): t_{R}=1.168 min; TLC (EtOAc): R_{f}=0.46

### EXAMPLE 107c

### {5-[5-(7-Chloro-1-propyl-1H-benzoimidazol-2-yl)-pyridin-2-ylamino]-pyridin-2-yl}-methanol

A solution of 5-[5-(7-chloro-1-propyl-1H-benzoimidazol-2-yl)-pyridin-2-ylamino]-pyridine-2-carboxylic acid methyl ester (1.2 g, 2.84 mmol) in anhydrous THF (50 ml) was cooled to 0° C. and treated with LiAlH₄ (167 mg, 4.27 mmol). The mixture was allowed to warm to RT and then stirred for 18 h. Another portion of LiAlH₄ (167 mg, 4.27 mmol) was then added and the mixture was stirred for another 1 h. The mixture was then cooled to 0° C. and treated dropwise with water (0.35 ml), aq. 1N NaOH solution (0.35 ml), and water (1.05 ml) again. The resulting suspension was filtered, the cake washed with EtOAc, and the filtrate was concentrated in vacuo. Purification by flash chromatography (DCM/MeOH 100:0 to 90:10), re-crystallization from EtOAc, and preparative HPLC (Column: Macherey-Nagel Nucelodur C-18; Gradient: Water+0.1% TFA/Acetonitrile+0.1% TFA from 90/10 to 20/80 over 38 min) provided the desired product (515 mg, 37%). UPLC (5-100% CH₃CN): t_{R}=0.944 min, TLC (DCM/MeOH 9:1): R_{f}=0.38.

### EXAMPLE 108

[5-(7-Chloro-1-isobutyl-1H-benzoimidazol-2-yl)-pyridin-2-yl]-p-tolyl-amine was prepared in analogy to the procedure described in example 81 starting from 7-Chloro-2-(6-chloro-pyridin-3-yl)-1-isobutyl-1H-benzoimidazole and p-toluidine.

HPLC (System 1, 0-100% CH₃CN): t_{R}=3.698 min; MS (LC-MS): 391 [M+1].

The starting material can be prepared as described below:

### 7-Chloro-2-(6-chloro-pyridin-3-yl)-1-isobutyl-1H-benzoimidazole

A mixture of 6-Chloro-nicotinoyl chloride (10.2 g, 58.0 mmol) and 3-Chloro-2N-isobutyl-benzene-1,2-diamine (11.0 g, 55.4 mmol) in DCM (300 ml) was stirred at 40° C. for 4 h. The reaction solution was washed with aq HCl (0.1 M) and NaHCO₃, dried over Na₂SO₄ and concentrated in vacuo. Recrystallisation from EtOAc afforded the desired product (10.2 g, 57%). HPLC (System 3, 30-100% CH₃CN): t_{R}=3.163 min, MS (LC-MS): 321 [M+1].

Following compounds can be prepared by analogy:

### EXAMPLE 109

### [5-(7-Chloro-1-isobutyl-1H-benzoimidazol-2-yl)-pyridin-2-yl]-(4-chloro-phenyl)-amine; HPLC (System 1, 0-100% CH₃CN): t_{R}=3.850 min; MS (LC-MS): 412 [M+1].

### EXAMPLE 110

### [5-(7-Chloro-1-isobutyl-1H-benzoimidazol-2-yl)-pyridin-2-yl]-(4-methoxy-phenyl)-amine; HPLC (System 3, 30-100% CH₃CN): t_{R}=2.593 min; MS (LC-MS): 407 [M+1].

### EXAMPLE 111

### [5-(7-Chloro-1-isobutyl-1H-benzoimidazol-2-yl)-pyridin-2-yl]-(6-methyl-pyridin-3-yl)-amine; HPLC (System 2, 10-100% CH₃CN): t_{R}=2.798 min; MS (LC-MS): 392 [M+1].

### EXAMPLE 112

### (4-Chloro-phenyl)-[5-(1-propyl-1H-benzoimidazol-2-yl)-thiazol-2-yl]-amine

A mixture of 2-(4-Chlorophenylamino)-5-thiazolecarboxylic acid (300 mg, 1.18 mmol) and N-propyl-benzene-1,2-diamine (212 mg, 1.41 mmol) in PPA (5 ml) was heated to 210° C. in a microwave oven for 20 min. The mixture was then poured onto water and the pH of the solution was adjusted to 10 with an aq. soln. of 2N NaOH, and the mixture was extracted with DCM. The combined org. layers were dried over Na₂SO₄ and concentrated in vacuo. Purification by flash chromatography (DCM/MeOH 100:0 to 94:6) afforded the desired product (16 mg, 4%). HPLC (System 2, 10-100% CH₃CN): t_{R}=3.270 min, MS (LC-MS): 369 [M+1].

Following compounds can be prepared by analogy:

### EXAMPLE 113

### (4-Chloro-phenyl)-[5-(1-propyl-1H-benzoimidazol-2-yl)-2H-pyrazol-3-yl]-amine; HPLC (System 2, 10-100% CH₃CN): t_{R}=3.202 min; MS (LC-MS): 352 [M+1].

### EXAMPLE 114

### [3-Chloro-5-(1-propyl-1H-benzoimidazol-2-yl)-pyridin-2-yl]-(4-methoxy-phenyl)-amine

A suspension of 2-(5,6-Dichloro-pyridin-3-yl)-1-propyl-1H-benzoimidazole (5.00 g, 16.3 mmol), p-anisidine (2.15 g, 17.1 mmol), rac-BINAP (1.04 mg, 1.64 mmol), Pd(OAc)₂ (780 mg, 1.63 mmol) and K₂CO₃ (11.4 g, 81.7 mmol) in toluene (200 ml) was heated to 100° C. for 48 h. The mixture was allowed to cool to RT and concentrated in vacuo. Purification by flash-chromatography (Hex/EtOAc 100:0 to 50:50) to afford the desired product (2.14 g, 37%). HPLC (System 3, 30-100% CH₃CN): t_{R}=2.309 min, MS (LC-MS): 393 [M+1].

The starting material can be prepared as described below:

### 2-(5,6-Dichloro-pyridin-3-yl)-1-propyl-1H-benzoimidazole

A mixture of 5,6-Dichloro-nicotinoyl chloride (26 g, 124 mmol) and N-Propyl-benzene-1,2-diamine (22.3 g, 148 mmol) in THF (300 ml) swas stirred at 60° C. for 18 h. The mixture was cooled to 0° C. and the precipitate filtered off to afford the desired product (22.2 mg, 52%). HPLC (System 3, 30-100% CH₃CN): t_{R}=1.952 min, MS (LC-MS): 307 [M+1].

### EXAMPLE 114b

### [3-chloro-5-(7-chloro-1-isobutyl-1H-benzoimidazol-2-yl)-pyridin-2-yl]-(4-methoxy-phenyl)-amine

A mixture of 7-chloro-2-(5,6-dichloro-pyridin-3-yl)-1-isobutyl-1H-benzoimidazole (2.00 g, 5.64 mmol), para-anisidine (850 mg, 6.76 mmol), Pd(OAc)₂ (253 mg, 1.13 mmol), rac-BINAP (702 mg, 1.13 mmol), and K₂CO₃ (3.90 g, 28.2 mmol) in Tol (50 ml) was heated to reflux for 18 h. The mixture was then concentrated in vacuo, taken up in EtOAc, filtered and the filtrated was concentrated in vacuo again. Purification by flash chromatography (Hex/EtOAc 100:0 to 75:25) and re-crystallization from Hex/EtOAc afforded the desired product (1.03 g, 41%). HPLC (System 3, 30-100% CH₃CN): t_{R}=3.275 min, TLC (Hex/EtOAc 3:1): R_{f}=0.36.

The starting material can be prepared as described below:

### 7-Chloro-2-(5,6-dichloro-pyridin-3-yl)-1-isobutyl-1H-benzoimidazole

A solution of 5,6-dichloro-nicotinoyl chloride (13.5 g, 64.1 mmol) and 3-chloro-N-2-isobutylbenzene-1,2-diamine (12.7 g, 63.9 mmol) in anhydrous THF (500 ml) was stirred at RT for 2 h. The mixture was then heated to 80° C. for 72 h, and then concentrated in vacuo. The residue was taken up in EtOAc, washed with a sat. aq NaHCO₃ solution, and the org. layer was dried over over Na₂SO₄, filtered and concentrated in vacuo. Purification by flash chromatography (Hex/EtOAc 80:20) provided 7-chloro-2-(5,6-dichloro-pyridin-3-yl)-1-isobutyl-1H-benzoimidazole (18.2 g, 80%). HPLC (System 3, 30-100% CH₃CN): t_{R}=3.731 min, TLC (Hex/EtOAc 4:1): R_{f}=0.42.

Following compound can be prepared by analogy:

### EXAMPLE 114c

### [3-chloro-5-(7-chloro-1-isobutyl-1H-benzoimidazol-2-yl)-pyridin-2-yl]-(6-methyl-pyridin-3-yl)-amine; HPLC (System 3, 30-100% CH₃CN): t_{R}=2.116 min; TLC (EtOAc): R_{f}=0.57

### EXAMPLE 114d

### [5-(7-fluoro-1-propyl-1H-benzoimidazol-2-yl)-pyridin-2-yl]-(4-methoxy-phenyl)-amine

A mixture of 2-(6-chloro-pyridin-3-yl)-7-fluoro-1-propyl-1H-benzoimidazole (410 mg, 1.26 mmol), para-anisidine (190 mg, 1.51 mmol), Pd(OAc)₂ (6 mg, 0.03 mmol), rac-BINAP (16 mg, 0.03 mmol), and Cs₂CO₃ (2.05 g, 6.29 mmol) in Tol (220 ml) was heated to reflux for 18 h. The mixture was allowed to cool to RT, and then filtered. The filtrate was concentrated in vacuo and purified by flash chromatography (Hex/EtOAc 100:0 to 75:25, 100:0 to 50:50, DCM/EtOAc 100:0 to 95:5) and re-crystallization from Hex gave the title compound (25 mg, 5%). HPLC (System 3, 30-100% CH₃CN): t_{R}=2.326 min, TLC (Hex/EtOAc 3:1): R_{f}=0.10.

The starting material can be prepared as described below:

### 2-(6-Chloro-pyridin-3-yl)-7-fluoro-1-propyl-1H-benzoimidazole

A solution of 6-chloro-nicotinoyl chloride (587 mg, 3.27 mmol) and 3-fluoro-N-2-propyl-benzene-1,2-diamine (550 mg, 3.27 mmol) in anhydrous THF (20 ml) was stirred at RT for 1 h, and then heated to reflux for 18 h. The mixture was then allowed to cool to RT, and then cooled to 0° C. The suspension was filtered and the filter cake was dried in vacuo at 60° C., affording 2-(6-chloro-pyridin-3-yl)-7-fluoro-1-propyl-1H-benzoimidazole (841 mg, 79%). HPLC (System 3, 30-100% CH₃CN): t_{R}=2.308 min, TLC (Hex/EtOAc 3:1): R_{f}=0.28.

### EXAMPLE 115

### [2-Chloro-4-(6-Fluoro-1-propyl-1H-benzoimidazol-2-yl)-phenyl]-(6-methyl-pyridin-3-yl)-amine

A mixture of 3-Chloro-4-(6-methyl-pyridin-3-ylamino)-benzoyl chloride (1.5 g, 4.54 mmol) and 4-Fluoro-N-2-propyl-benzene-1,2-diamine (801 mg, 4.76 mmol) in THF (15 ml) was stirred at 60° C. for 15 h. The reaction mixture was then concentrated in vacuo diluted with EtOAc and washed twice with aq. NaHCO₃. The organic layer was dried over Na₂SO₄, filtered and concentrated in vacuo. The crude intermediated was then dissolved in toluene (15 ml) and p-TsOH (100 mg) was added. After stirring the reaction mixture at 110° C. for 24 h in a Dean-Stark apparatus, the solution was cooled to RT, washed with NaHCO₃ and water, dried over Na₂SO₄, filtered and concentrated in vacuo. Purification by flash chromatography (Hex/EtOAc 100:0 to 10:100) afforded the desired product (275 mg, 15%). HPLC (System 3, 30-100% CH₃CN): t_{R}=0.479 min, MS (ES+): 395 [M+1]

The starting materials can be prepared as described hereafter:

### 3-Chloro-4-(6-methyl-pyridin-3-ylamino)-benzoyl Chloride

A mixture of 3-Chloro-4-(6-methyl-pyridin-3-ylamino)-benzoic acid (2.5 g, 9.52 mmol) and SOCl₂ (0.762 ml, 10.5 mmol) in toluene (15 ml) was treated with DMF (14.7 μl, 0.191 mmol) at RT and the mixture was then heated to 80° C. for 1 h. The mixture was allowed to cool to RT and then concentrated in vacuo to give the crude acid chloride (3.07 g, 100%).

### 3-Chloro-4-(6-methyl-pyridin-3-ylamino)-benzoic Acid

A solution of 3-Chloro-4-(6-methyl-pyridin-3-ylamino)-benzoic acid methyl ester (9.0 g, 32.5 mmol) in MeOH (120 ml) was treated slowly with 1N aq. NaOH (120 ml, 120 mmol). Resulting solution was stirred for 2 h at 70° C., concentrated in vacuo to remove the MeOH and the pH adjusted to 6-7 by addition of 2N aq. HCl. The desired 3-Chloro-4-(6-methyl-pyridin-3-ylamino)-benzoic acid precipitated and was collected by filtration (7.21 g, 84%). HPLC (System 2, 10-100% CH₃CN): t_{R}=2.012 min, MS (ES+): 262 [M+1]

### 3-Chloro-4-(6-methyl-pyridin-3-ylamino)-benzoic Acid Methyl Ester

A suspension of 4-Bromo-3-chloro-benzoic acid methyl ester (7.0 g, 28.1 mmol), 3-amino-6-methylpyridine (3.19 g, 29.5 mmol), rac-BINAP (1.75 g, 2.81 mmol), Pd₂(dba)₃ (0.629 g, 2.8 mmol) and K₂CO₃ (19.4 g, 140 mmol) in toluene (250 mL) was heated to 80° C. for 16 h. The mixture was allowed to cool to RT and washed twice with aq. NaHCO₃. The organic layer was dried over Na₂SO₄, filtered and concentrated in vacuo to afford crude 3-Chloro-4-(6-methyl-pyridin-3-ylamino)-benzoic acid methyl ester (9.12 g, 100%) which was used without further purification. HPLC (System 1, 0-100% CH₃CN): t_{R}=2.867 min, MS (ES+): 277 [M+1]

Following compound can be prepared in analogy to the sequence described above by using 3-Chloro-N-2-isobutyl-benzene-1,2-diamine instead of 4-Fluoro-N-2-propyl-benzene-1,2-diamine:

### EXAMPLE 116

### [2-Chloro-4-(7-Chloro-1-isobutyl-1H-benzoimidazol-2-yl)-phenyl]-(6-methyl-pyridin-3-yl)-amine; UPLC (5-100% CH₃CN): t_{R}=1.121 min; MS (ES+): 426 [M+1].

### Biological Testing.

Activity of compounds of the present invention was examined by determination to what extent the agonist induced elevation of the inositol phosphate turnover is inhibited as described by T. Knoepfel et al., Eur. J. Pharmacol. Vol. 288, pages 389-392 (1994), L. P. Daggett et al., Neuropharm. Vol. 67, pages 58-63 (1996) and references cited therein.

The table below represents percentages of inhibition of the glutamate induced elevation of the inositol phosphate turnover at a concentration of 0.1 μM.

| | mGluR5-activity in [%] |
|---|---|
| Compound | inhibition at 0.1 μM |
| 19 | 57 |
| 20 | 98 |
| 21 | 65 |
| 22 | 97 |
| 23 | 84 |
| 24 | 101 |
| 25 | 97 |
| 26 | 98 |
| 29 | 99 |
| 30 | 99 |
| 31 | 90 |
| 34b | 97 |
| 45 | 91 |
| 47 | 100 |
| 48 | 101 |
| 52 | 99 |
| 53 | 95 |
| 54 | 97 |
| 55 | 63 |
| 56 | 100 |
| 57 | 93 |
| 58 | 40 |
| 59 | 51 |
| 60 | 87 |
| 61 | 96 |
| 62 | 99 |
| 63 | 28 |
| 64 | 17 |
| 65 | 92 |
| 66 | 93 |
| 67 | 42 |
| 68 | 43 |
| 69 | 32 |
| 70 | 90 |
| 71 | 20 |
| 71b | 100 |
| 72 | 96 |
| 73 | 98 |
| 74 | 99 |
| 75 | 99 |
| 75b | 99 |
| 75c | 100 |
| 75d | 100 |
| 75e | 100 |
| 75f | 92 |
| 78 | 99 |
| 79 | 65 |
| 80 | 90 |
| 81 | 96 |
| 82 | 88 |
| 83 | 52 |
| 86 | 60 |
| 87 | 47 |
| 88 | 97 |
| 90 | 102 |
| 91 | 100 |
| 92 | 99 |
| 93 | 34 |
| 94 | 97 |
| 95 | 63 |
| 96 | 85 |
| 97 | 98 |
| 98 | 103 |
| 99 | 99 |
| 100 | 100 |
| 101 | 95 |
| 102 | 90 |
| 103 | 97 |
| 104 | 84 |
| 105 | 85 |
| 106 | 70 |
| 107 | 64 |
| 108 | 98 |
| 109 | 91 |
| 110 | 95 |
| 111 | 97 |
| 114 | 99 |
| 114b | 100 |
| 114c | 111 |
| 114d | 82 |
| 115 | 99 |
| 116 | 100 |

Furthermore, the invention provides a compound selected from:
[3-Chloro-5-(1-methyl-1H-benzoimidazol-2-yl)-pyridin-2-yl]-(4-chloro-phenyl)-amine,
[3-Chloro-5-(1-ethyl-1H-benzoimidazol-2-yl)-pyridin-2-yl]-(4-chloro-phenyl)-amine,
(4-Chloro-phenyl)-[3-chloro-5-(1-propyl-1H-benzoimidazol-2-yl)-pyridin-2-yl]-amine,
[5-(1-Butyl-1H-benzoimidazol-2-yl)-3-chloro-pyridin-2-yl]-(4-chloro-phenyl)-amine,
[3-Chloro-5-(1-isopropyl-1H-benzoimidazol-2-yl)-pyridin-2-yl]-(4-chloro-phenyl)-amine,
[3-Chloro-5-(1-isobutyl-1H-benzoimidazol-2-yl)-pyridin-2-yl]-(4-chloro-phenyl)-amine,
[3-Chloro-5-(1-cyclopropylmethyl-1H-benzoimidazol-2-yl)-pyridin-2-yl]-(4-chloro-phenyl)-amine,
(4-Chloro-phenyl)-[3-chloro-5-(1-propyl-1H-imidazo[4,5-c]pyridin-2-yl)-pyridin-2-yl]-amine,
[3-Chloro-5-(5-fluoro-1-methyl-1H-benzoimidazol-2-yl)-pyridin-2-yl]-(4-chloro-phenyl)-amine,
[3-Chloro-5-(6-fluoro-1-methyl-1H-benzoimidazol-2-yl)-pyridin-2-yl]-(4-chloro-phenyl)-amine,
[3-Chloro-5-(1-ethyl-5-fluoro-1H-benzoimidazol-2-yl)-pyridin-2-yl]-(4-chloro-phenyl)-amine,
[3-Chloro-5-(1-ethyl-6-fluoro-1H-benzoimidazol-2-yl)-pyridin-2-yl]-(4-chloro-phenyl)-amine,
[3-Chloro-5-(5-fluoro-1-propyl-1H-benzoimidazol-2-yl)-pyridin-2-yl]-(4-chloro-phenyl)-amine,
[3-Chloro-5-(6-fluoro-1-propyl-1H-benzoimidazol-2-yl)-pyridin-2-yl]-(4-chloro-phenyl)-amine,
[(4-Chloro-phenyl)-[3-chloro-5-(3-propyl-3H-imidazo[4,5-b]pyridin-2-yl)-pyridin-2-yl]-amine,
[3-Chloro-5-(1-ethyl-1H-benzoimidazol-2-yl)-pyridin-2-yl]-(6-methyl-pyridin-3-yl)-amine,
[3-Chloro-5-(1-propyl-1H-benzoimidazol-2-yl)-pyridin-2-yl]-(6-methyl-pyridin-3-yl)-amine,
[5-(1-Butyl-1H-benzoimidazol-2-yl)-3-chloro-pyridin-2-yl]-(6-methyl-pyridin-3-yl)-amine,
[3-Chloro-5-(1-pentyl-1H-benzoimidazol-2-yl)-pyridin-2-yl]-(6-methyl-pyridin-3-yl)-amine,
[3-Chloro-5-(1-isobutyl-1H-benzoimidazol-2-yl)-pyridin-2-yl]-(6-methyl-pyridin-3-yl)-amine,
[3-Chloro-5-(1-propyl-5-trifluoromethyl-1H-benzoimidazol-2-yl)-pyridin-2-yl]-(6-methyl-pyridin-3-yl)-amine,
[3-Chloro-5-(5-methyl-1-propyl-1H-benzoimidazol-2-yl)-pyridin-2-yl]-(6-methyl-pyridin-3-yl)-amine,
[3-Chloro-5-(6-methyl-1-propyl-1H-benzoimidazol-2-yl)-pyridin-2-yl]-(6-methyl-pyridin-3-yl)-amine,
[3-Chloro-5-(7-methyl-1-propyl-1H-benzoimidazol-2-yl)-pyridin-2-yl]-(6-methyl-pyridin-3-yl)-amine,
[3-Chloro-5-(4-methyl-1-propyl-1H-benzoimidazol-2-yl)-pyridin-2-yl]-(6-methyl-pyridin-3-yl)-amine,
[3-Chloro-5-(6,7-dimethyl-1-propyl-1H-benzoimidazol-2-yl)-pyridin-2-yl]-(6-methyl-pyridin-3-yl)-amine,
[3-Chloro-5-(5,7-dimethyl-1-propyl-1H-benzoimidazol-2-yl)-pyridin-2-yl]-(6-methyl-pyridin-3-yl)-amine,
[3-Chloro-5-(5,6-dimethyl-1-propyl-1H-benzoimidazol-2-yl)-pyridin-2-yl]-(6-methyl-pyridin-3-yl)-amine,
[3-Chloro-5-(4,7-dimethyl-1-propyl-1H-benzoimidazol-2-yl)-pyridin-2-yl]-(6-methyl-pyridin-3-yl)-amine,
[3-Chloro-5-(5,6-difluoro-1-propyl-1H-benzoimidazol-2-yl)-pyridin-2-yl]-(6-methyl-pyridin-3-yl)-amine,
[3-Chloro-5-(5,6-dihydro-4H-imidazo[4,5,1-ij]quinolin-2-yl)-pyridin-2-yl]-(6-methyl-pyridin-3-yl)-amine,
[3-Chloro-5-(1-propyl-1H-benzoimidazol-2-yl)-pyridin-2-yl]-phenyl-amine,
[3-Chloro-5-(1-propyl-1H-benzoimidazol-2-yl)-pyridin-2-yl]-pyridin-4-yl-amine,
[3-Chloro-5-(1-propyl-1H-benzoimidazol-2-yl)-pyridin-2-yl]-p-tolyl-amine,
[3-Chloro-5-(7-chloro-5-iodo-1-propyl-1H-benzoimidazol-2-yl)-pyridin-2-yl]-(6-methyl-pyridin-3-yl)-amine,
[3-Chloro-5-(1-ethyl-5-fluoro-1H-benzoimidazol-2-yl)-pyridin-2-yl]-(6-methyl-pyridin-3-yl)-amine,
[3-Chloro-5-(1-ethyl-6-fluoro-1H-benzoimidazol-2-yl)-pyridin-2-yl]-(6-methyl-pyridin-3-yl)-amine,
[3-Chloro-5-(5-fluoro-1-propyl-1H-benzoimidazol-2-yl)-pyridin-2-yl]-(6-methyl-pyridin-3-yl)-amine,
[3-Chloro-5-(6-fluoro-1-propyl-1H-benzoimidazol-2-yl)-pyridin-2-yl]-(6-methyl-pyridin-3-yl)-amine,
[5-(1-Butyl-5-fluoro-1H-benzoimidazol-2-yl)-3-chloro-pyridin-2-yl]-(6-methyl-pyridin-3-yl)-amine,
[5-(1-Butyl-6-fluoro-1H-benzoimidazol-2-yl)-3-chloro-pyridin-2-yl]-(6-methyl-pyridin-3-yl)-amine,
[3-Chloro-5-(4-fluoro-1-propyl-1H-benzoimidazol-2-yl)-pyridin-2-yl]-(6-methyl-pyridin-3-yl)-amine,
[3-Chloro-5-(7-fluoro-1-propyl-1H-benzoimidazol-2-yl)-pyridin-2-yl]-(6-methyl-pyridin-3-yl)-amine,
[3-Chloro-5-(4,5-difluoro-1-propyl-1H-benzoimidazol-2-yl)-pyridin-2-yl]-(6-methyl-pyridin-3-yl)-amine,
[3-Chloro-5-(6,7-difluoro-1-propyl-1H-benzoimidazol-2-yl)-pyridin-2-yl]-(6-methyl-pyridin-3-yl)-amine,
[3-Chloro-5-(5-chloro-1-propyl-1H-benzoimidazol-2-yl)-pyridin-2-yl]-(6-methyl-pyridin-3-yl)-amine,
[3-Chloro-5-(6-chloro-1-propyl-1H-benzoimidazol-2-yl)-pyridin-2-yl]-(6-methyl-pyridin-3-yl)-amine,
[3-Chloro-5-(4-chloro-1-propyl-1H-benzoimidazol-2-yl)-pyridin-2-yl]-(6-methyl-pyridin-3-yl)-amine,
[3-Chloro-5-(7-chloro-1-propyl-1H-benzoimidazol-2-yl)-pyridin-2-yl]-(6-methyl-pyridin-3-yl)-amine,
[3-Chloro-5-(4,6-dichloro-1-propyl-1H-benzoimidazol-2-yl)-pyridin-2-yl]-(6-methyl-pyridin-3-yl)-amine,
[3-Chloro-5-(5,7-dichloro-1-propyl-1H-benzoimidazol-2-yl)-pyridin-2-yl]-(6-methyl-pyridin-3-yl)-amine,
[3-Chloro-5-(5,6-dichloro-1-propyl-1H-benzoimidazol-2-yl)-pyridin-2-yl]-(6-methyl-pyridin-3-yl)-amine,
[3-Chloro-5-(7-chloro-1-propyl-1H-benzoimidazol-2-yl)-pyridin-2-yl]-(4-chloro-phenyl)-amine,
[3-Chloro-5-(7-chloro-1-propyl-1H-benzoimidazol-2-yl)-pyridin-2-yl]-(2-fluoro-phenyl)-amine,
[3-Chloro-5-(7-chloro-1-propyl-1H-benzoimidazol-2-yl)-pyridin-2-yl]-(4-fluoro-phenyl)-amine,
[3-Chloro-5-(7-chloro-1-propyl-1H-benzoimidazol-2-yl)-pyridin-2-yl]-(3-fluoro-phenyl)-amine,
4-[3-Chloro-5-(7-chloro-1-propyl-1H-benzoimidazol-2-yl)-pyridin-2-ylamino]-benzonitrile,
[3-Chloro-5-(7-chloro-1-propyl-1H-benzoimidazol-2-yl)-pyridin-2-yl]-(3,4-difluoro-phenyl)-amine,
[3-Chloro-5-(7-chloro-1-propyl-1H-benzoimidazol-2-yl)-pyridin-2-yl]-(2,3-difluoro-phenyl)-amine,
[3-Chloro-5-(7-chloro-1-propyl-1H-benzoimidazol-2-yl)-pyridin-2-yl]-pyridin-4-yl-amine,
[3-Chloro-5-(7-chloro-1-propyl-1H-benzoimidazol-2-yl)-pyridin-2-yl]-(6-methyl-pyridazin-3-yl)-amine,
[3-Chloro-5-(7-chloro-1-propyl-1H-benzoimidazol-2-yl)-pyridin-2-yl]-quinoxalin-6-yl-amine,
[3-Chloro-5-(7-chloro-1-propyl-1H-benzoimidazol-2-yl)-pyridin-2-yl]-(5-methyl-pyrazin-2-yl)-amine,
[3-Chloro-5-(7-chloro-1-propyl-1H-benzoimidazol-2-yl)-pyridin-2-yl]-quinolin-3-yl-amine,
[3-Chloro-5-(7-chloro-1-propyl-1H-benzoimidazol-2-yl)-pyridin-2-yl]-quinolin-6-yl-amine,
[3-Chloro-5-(6-fluoro-1-propyl-1H-benzoimidazol-2-yl)-pyridin-2-yl]-p-tolyl-amine,
[3-Chloro-5-(6-fluoro-1-propyl-1H-benzoimidazol-2-yl)-pyridin-2-yl]-(4-methoxy-phenyl)-amine,
[3-Chloro-5-(7-chloro-1-propyl-1H-benzoimidazol-2-yl)-pyridin-2-yl]-(2,6-difluoro-phenyl)-amine,
N-5-[3-Chloro-5-(6-fluoro-1-propyl-1H-benzoimidazol-2-yl)-pyridin-2-yl]-N-2, N-2-dimethyl-pyridine-2,5-diamine,
N-5-[3-Chloro-5-(1-propyl-1H-benzoimidazol-2-yl)-pyridin-2-yl]-N-2, N-2-dimethyl-pyridine-2,5-diamine,
N-5-[3-Chloro-5-(7-chloro-1-propyl-1H-benzoimidazol-2-yl)-pyridin-2-yl]-N-2,N-2-dimethyl-pyridine-2,5-diamine,
N-{5-[3-Chloro-5-(6-fluoro-1-propyl-1H-benzoimidazol-2-yl)-pyridin-2-ylamino]-pyridin-2-yl}-acetamide,
N-5-[3-Chloro-5-(6-fluoro-1-propyl-1H-benzoimidazol-2-yl)-pyridin-2-yl]-pyridine-2,5-diamine,
[3-Chloro-5-(7-chloro-1-propyl-1H-benzoimidazol-2-yl)-pyridin-2-yl]-pyrazin-2-yl-amine,
[3-Chloro-5-(7-chloro-1-propyl-1H-benzoimidazol-2-yl)-pyridin-2-yl]-(3-methyl-isoxazol-5-yl)-amine,
[3-Chloro-5-(7-chloro-1-propyl-1H-benzoimidazol-2-yl)-pyridin-2-yl]-(6-methoxy-pyridin-3-yl)-amine,
[3-Chloro-5-(7-chloro-1-propyl-1H-benzoimidazol-2-yl)-pyridin-2-yl]-(4-methoxy-phenyl)-amine,
5-[3-Chloro-5-(7-chloro-1-propyl-1H-benzoimidazol-2-yl)-pyridin-2-ylamino]-pyridine-2-carbonitrile,
N-{5-[3-Chloro-5-(7-chloro-1-propyl-1H-benzoimidazol-2-yl)-pyridin-2-ylamino]-pyridin-2-yl}acetamide,
5-[3-Chloro-5-(7-chloro-1-propyl-1H-benzoimidazol-2-yl)-pyridin-2-ylamino]-pyridine-2-carboxylic acid methyl ester,
{5-[3-Chloro-5-(7-chloro-1-propyl-1H-benzoimidazol-2-yl)-pyridin-2-ylamino]-pyridin-2-yl}-methanol,
[6-(6-Fluoro-1-propyl-1H-benzoimidazol-2-yl)-pyridin-3-yl]-(6-methyl-pyridin-3-yl)-amine,
(4-Chloro-phenyl)-[4-(6-fluoro-1-propyl-1H-benzoimidazol-2-yl)-phenyl]-amine,
[4-(7-Chloro-1-propyl-1H-benzoimidazol-2-yl)-phenyl]-(6-methyl-pyridin-3-yl)-amine,
[4-(6-Fluoro-1-propyl-1H-benzoimidazol-2-yl)-phenyl]-(6-methyl-pyridin-3-yl)-amine,
[4-(7-Chloro-1-propyl-1H-benzoimidazol-2-yl)-phenyl]-p-tolyl-amine,
(4-Chloro-phenyl)-[4-(7-chloro-1-propyl-1H-benzoimidazol-2-yl)-phenyl]-amine,
[4-(7-Chloro-1-propyl-1H-benzoimidazol-2-yl)-phenyl]-(4-methoxy-phenyl)-amine,
[4-(6-Fluoro-1-propyl-1H-benzoimidazol-2-yl)-phenyl]-p-tolyl-amine,
(4-Chloro-phenyl)-[6-(1-propyl-1H-benzoimidazol-2-yl)-pyridazin-3-yl]-amine,
(6-Methyl-pyridin-3-yl)-[6-(1-propyl-1H-benzoimidazol-2-yl)-pyridazin-3-yl]-amine,
[6-(7-Chloro-1-propyl-1H-benzoimidazol-2-yl)-pyridazin-3-yl]-(4-methoxy-phenyl)-amine,
(4-Chloro-phenyl)-[6-(7-chloro-1-propyl-1H-benzoimidazol-2-yl)-pyridazin-3-yl]-amine,
[6-(7-Chloro-1-propyl-1H-benzoimidazol-2-yl)-pyridazin-3-yl]-(6-methyl-pyridin-3-yl)-amine,
(6-Methyl-pyridin-3-yl)-[5-(1-propyl-1H-benzoimidazol-2-yl)-pyrimidin-2-yl]-amine,
[5-(7-Chloro-1-propyl-1H-benzoimidazol-2-yl)-pyrimidin-2-yl]-(4-methoxy-phenyl)-amine,
(4-Chloro-phenyl)-[5-(7-chloro-1-propyl-1H-benzoimidazol-2-yl)-pyrimidin-2-yl]-amine,
[5-(7-Chloro-1-propyl-1H-benzoimidazol-2-yl)-pyrimidin-2-yl]-(6-methyl-pyridin-3-yl)-amine,
[5-(6-Fluoro-1-propyl-1H-benzoimidazol-2-yl)-pyrimidin-2-yl]-(4-methoxy-phenyl)-amine,
[5-(7-Chloro-5-iodo-1-propyl-1H-benzoimidazol-2-yl)-pyrimidin-2-yl]-(6-methyl-pyridin-3-yl)-amine,
[2-Chloro-4-(7-chloro-1-propyl-1H-benzoimidazol-2-yl)-phenyl]-p-tolyl-amine,
[2-Chloro-4-(7-chloro-1-propyl-1H-benzoimidazol-2-yl)-phenyl]-(4-chloro-phenyl)-amine,
[2-Chloro-4-(7-chloro-1-propyl-1H-benzoimidazol-2-yl)-phenyl]-(4-methoxy-phenyl)-amine,
[2-Chloro-4-(7-chloro-1-propyl-1H-benzoimidazol-2-yl)-phenyl]-(6-methyl-pyridin-3-yl)-amine,
[5-(7-Chloro-1-propyl-1H-benzoimidazol-2-yl)-pyridin-2-yl]-(4-methoxy-phenyl)-amine,
[5-(7-Chloro-1-propyl-1H-benzoimidazol-2-yl)-pyridin-2-yl]-(6-methyl-pyridin-3-yl)-amine,
(4-Chloro-phenyl)-[5-(7-chloro-1-propyl-1H-benzoimidazol-2-yl)-pyridin-2-yl]-amine,
(6-Methyl-pyridin-3-yl)-[5-(1-propyl-1H-benzoimidazol-2-yl)-pyridin-2-yl]-amine,
[5-(7-Chloro-1-propyl-1H-benzoimidazol-2-yl)-pyridin-2-yl]-p-tolyl-amine,
(4-Chloro-phenyl)-[5-(6-fluoro-1-propyl-1H-benzoimidazol-2-yl)-pyridin-2-yl]-amine,
[5-(6-Fluoro-1-propyl-1H-benzoimidazol-2-yl)-pyridin-2-yl]-(6-methyl-pyridin-3-yl)-amine,
[5-(6-Fluoro-1-propyl-1H-benzoimidazol-2-yl)-pyridin-2-yl]-p-tolyl-amine,
[5-(6-Fluoro-1-propyl-1H-benzoimidazol-2-yl)-pyridin-2-yl]-(4-methoxy-phenyl)-amine,
5-[5-(7-Chloro-1-propyl-1H-benzoimidazol-2-yl)-pyridin-2-ylamino]-pyridine-2-carboxylic acid methyl ester,
{5-[5-(7-Chloro-1-propyl-1H-benzoimidazol-2-yl)-pyridin-2-ylamino]-pyridin-2-yl}-methanol,
[5-(7-Chloro-1-isobutyl-1H-benzoimidazol-2-yl)-pyridin-2-yl]-p-tolyl-amine,
[5-(7-Chloro-1-isobutyl-1H-benzoimidazol-2-yl)-pyridin-2-yl]-(4-chloro-phenyl)-amine,
[5-(7-Chloro-1-isobutyl-1H-benzoimidazol-2-yl)-pyridin-2-yl]-(4-methoxy-phenyl)-amine,
[5-(7-Chloro-1-isobutyl-1H-benzoimidazol-2-yl)-pyridin-2-yl]-(6-methyl-pyridin-3-yl)-amine,
(4-Chloro-phenyl)-[5-(1-propyl-1H-benzoimidazol-2-yl)-thiazol-2-yl]-amine,
(4-Chloro-phenyl)-[5-(1-propyl-1H-benzoimidazol-2-yl)-2H-pyrazol-3-yl]-amine,
[3-Chloro-5-(1-propyl-1H-benzoimidazol-2-yl)-pyridin-2-yl]-(4-methoxy-phenyl)-amine,
[3-chloro-5-(7-chloro-1-isobutyl-1H-benzoimidazol-2-yl)-pyridin-2-yl]-(4-methoxy-phenyl)-amine,
[3-chloro-5-(7-chloro-1-isobutyl-1H-benzoimidazol-2-yl)-pyridin-2-yl]-(6-methyl-pyridin-3-yl)-amine,
[5-(7-fluoro-1-propyl-1H-benzoimidazol-2-yl)-pyridin-2-yl]-(4-methoxy-phenyl)-amine,
[2-Chloro-4-(6-Fluoro-1-propyl-1H-benzoimidazol-2-yl)-phenyl]-(6-methyl-pyridin-3-yl)-amine, and
[2-Chloro-4-(7-Chloro-1-isobutyl-1H-benzoimidazol-2-yl)-phenyl]-(6-methyl-pyridin-3-yl)-amine.

1. A compound of formula (I): 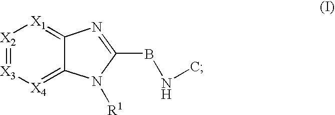
wherein:
X₁, X₂, X₃, X₄ each independently represent CR² or N, provided that at least two of X₁, X₂, X₃ and X₄ are CR²;
each R² independently is hydrogen, halogen, hydroxyl, nitro, cyano, formyl, carboxy, carboxamido, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino, (C₁₋₆alkoxycarbonyl)amino, C₁₋₆alkoxy, C₁₋₆alkoxycarbonyl, sulphonate, sulphate, phosphate, quartenary ammonium, phosphonate, guanidimium, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₃₋₁₂cycloalkyl, C₃₋₁₂halogencycloalkyl, C₁₋₆alkyl(C₃₋₁₂cycloalkyl), C₃₋₁₂cycloalkyl(C₁₋₆alkyl), C₃₋₁₂cycloalkyloxy, C₂₋₆alkenyl, C₂₋₆halogenalkenyl, C₂₋₆alkynyl or C₂₋₆halogenalkynyl;
R¹ is C₁₋₆alkyl, C₁₋₆halogenalkyl, C₃₋₁₂cycloalkyl, C₃₋₁₂halogencycloalkyl, C₁₋₆alkyl(C₃₋₁₂cycloalkyl) or C₃₋₁₂cycloalkyl(C₁₋₆alkyl);
or, when X₄ is CR², R¹, R² and the nitrogen and two carbon atoms, to which R¹ and R² are bound, may form together a 5- to 8-membered heterocyclic ring system, which may be aromatic or partially saturated and which may contain from 1 to 2 further hetero atoms selected from nitrogen, oxygen and sulfur, and wherein the heterocyclic ring system itself may be substituted once or more than once by R^{a};
each R^{a} independently is halogen, nitro, cyano, formyl, carboxy, carboxamido, hydroxyl, amino, (C₁₋₆alkyl)amino, di-(C₁₋₆alkyl)amino, (C₁₋₆alkoxycarbonyl)amino, C₁₋₆alkoxy, C₁₋₆alkoxycarbonyl, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₂₋₆alkenyl, C₂₋₆halogenalkenyl, C₂₋₆alkynyl or C₂₋₆halogenalkynyl;
B is 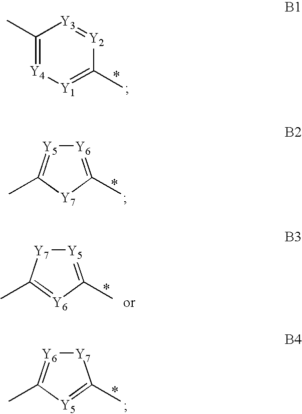
wherein the bond marked with the asterisk is attached to the group —NH—C;
Y₁, Y₂, Y₃ and Y₄ each independently represent CR³ or N, provided that at least one of Y₁, Y₂, Y₃ and Y₄ is CR³;
Y₅ and Y₆ each independently represent CR³ or N, provided that at least one of Y₅ and Y₆ is CR³;
Y₇ is O, S or N(R^{3a});
each R³ independently is hydrogen, halogen, hydroxyl, nitro, cyano, formyl, carboxy, carboxamido, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino, (C₁₋₆alkoxycarbonyl)amino, C₁₋₆alkoxy, C₁₋₆alkoxycarbonyl, sulphonate, sulphate, phosphate, quartenary ammonium, phosphonate, guanidimium, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₃₋₁₂cycloalkyl, C₃₋₁₂halogencycloalkyl, C₁₋₆alkyl(C₃₋₁₂cycloalkyl), C₃₋₁₂cycloalkyl(C₁₋₆alkyl), C₃₋₁₂cycloalkyloxy, C₂₋₆alkenyl, C₂₋₆halogenalkenyl, C₂₋₆alkynyl or C₂₋₆halogenalkynyl;
R^{3a} is hydrogen, C₁₋₆alkyl, C₁₋₆halogenalkyl, C₃₋₁₂cycloalkyl, C₃₋₁₂halogencycloalkyl, C₁₋₆alkyl(C₃₋₁₂cycloalkyl) or C₃₋₁₂cycloalkyl(C₁₋₆alkyl);
C is a 5- to 12-membered aromatic ring system, which may be monocyclic or fused polycyclic, which may contain from 1 to 3 hetero atoms selected from nitrogen, oxygen and sulfur, and wherein the ring system itself may be substituted once or more than once by R^{b};
each R^{b} independently is halogen, hydroxyl, nitro, cyano, formyl, carboxy, carboxamido, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino, (C₁₋₆alkoxycarbonyl)amino, (C₁₋₆alkylcarbonyl)amino, C₁₋₆alkoxy, C₁₋₆alkoxycarbonyl, sulphonate, sulphate, phosphate, quartenary ammonium, phosphonate, guanidimium, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₃₋₁₂cycloalkyl, C₃₋₁₂halogencycloalkyl, C₁₋₆alkyl(C₃₋₁₂cycloalkyl), C₃₋₁₂cycloalkyl(C₁₋₆alkyl), C₃₋₁₂cycloalkyloxy, C₂₋₆alkenyl, C₂₋₆halogenalkenyl, C₂₋₆alkynyl or C₂₋₆halogenalkynyl;
or two groups R^{b} bound to adjacent carbon atoms of the ring system together are a C₃₋₆alkandiyl group, wherein a carbon atom may be substituted by —O—, —S—, —N(R^{c})—, —C(═O)—, —C(═S)—, —C(═NR^{d})—, —S(═O)— or —SO₂—, and wherein the group may be substituted once or more than once by R^{e};
each R^{c}, R^{d} or R^{e} independently is halogen or C₁₋₆alkyl;
or two groups R^{b} bound to adjacent carbon atoms of the ring system together are a group —O—(C(R^{f})₂), —O—;
each R^{f} independently is hydrogen, halogen or C₁₋₆alkyl;
n is 1 or 2;
and salts, solvates, hydrates and N-oxides thereof.

2. The compound of formula (I) according to claim 1, wherein C is: 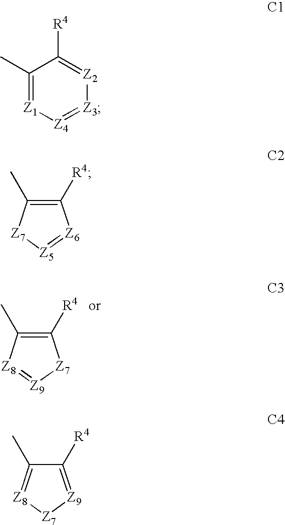
wherein
Z₁, Z₂, Z₃ and Z₄ each independently represent CR⁴ or N, provided that at least two of Z₁, Z₂, Z₃ and Z₄ are CR⁴; and
Z₅ and Z₆ each independently represent CR⁴ or N, provided that at least one of Z₅ and Z₆ is CR⁴;
Z₈ and Z₉ each independently represent CR⁴ or N, provided that at least one of Z₈ and Z₉ is CR⁴;
Z₇ is O, S or N(R^{4a});
each R⁴ individually represents hydrogen, halogen, hydroxyl, nitro, cyano, formyl, carboxy, carboxamido, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino, (C₁₋₆alkoxycarbonyl)amino, (C₁₋₆alkylcarbonyl)amino, C₁₋₄alkoxy, C₁₋₆alkoxycarbonyl, sulphonate, sulphate, phosphate, quartenary ammonium, phosphonate, guanidimium, C₁₋₆alkyl, C₁₋₆halogenalkyl, C₁₋₆hydroxyalkyl, C₁₋₆alkylcarbonyl(C₁₋₆alkyl), C₁₋₆alkoxy(C₁₋₆alkyl), C₁₋₆alkoxycarbonyl(C₁₋₆alkyl), C₁₋₆aminoalkyl, C₁₋₆alkylamino(C₁₋₆alkyl), di-(C₁₋₆alkyl)amino(C₁₋₆alkyl), C₃₋₁₂cycloalkyl, C₃₋₁₂halogencycloalkyl, C₁₋₆alkyl(C₃₋₁₂cycloalkyl), C₃₋₁₂cycloalkyl(C₁₋₆alkyl), C₃₋₁₂cycloalkyloxy, C₂₋₆alkenyl, C₂₋₆halogenalkenyl, C₂₋₆alkynyl or C₂₋₆halogenalkynyl;
R^{4a} is hydrogen, C₁₋₆alkyl, C₁₋₆halogenalkyl, C₃₋₁₂cycloalkyl, C₃₋₁₂halogencycloalkyl, C₁₋₆alkyl(C₃₋₁₂cycloalkyl) or C₃₋₁₂cycloalkyl(C₁₋₆alkyl);
or, when Z₂ and Z₃ are both CR⁴, these two R⁴ groups may, together with the two carbon atoms to which they are attached, form a 5- or 6-membered aryl or aromatic heterocyclic ring system, which may be substituted once or more than once by halogen, C₁₋₆alkyl or C₁₋₆halogenalkyl;
or, when Z₅ and Z₆ are both CR⁴, these two R⁴ groups may, together with the two carbon atoms to which they are attached, form a 5- or 6-membered aryl or aromatic heterocyclic ring system, which may be substituted once or more than once by halogen, C₁₋₆alkyl or C₁₋₆halogenalkyl.

3. The compound of formula (I) according to claim 1, wherein B is B1.

4. The compound of formula (I) according to claim 1, wherein C is C1.

5. The compound of formula (I) having the formula (III): 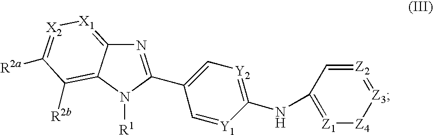
wherein
X₁, X₂ each independently represent CR² or N;
R^{2a}, R^{2b} each independently represent a group chosen from hydrogen, halogen, hydroxy, C₁₋₆alkyl, C₁₋₆halogenalkyl, C₃₋₁₂cycloalkyl, amino, C₁₋₆alkoxy, C₃₋₁₂cycloalkyloxy, sulphonate, sulphate, phosphate, quartenary ammonium, phosphonate and guanidimium;
R¹ is C₁₋₆alkyl or C₃₋₁₂cycloalkyl;
Y₁ and Y₂ each independently represent CR³ or N;
R³ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino or cyano;
Z₁, Z₂, Z₃, Z₄ each independently represent CR⁴ or N, provided that at least one is CR⁴; and
R⁴ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino or di(C₃₋₁₂cycloalkyl)amino, cyano, C₁₋₆hydroxyalkyl, C₁₋₆alkoxycarbonyl or C₁₋₆alkylcarbonylamino.

6. The compound of formula (I) having the formula (IV): 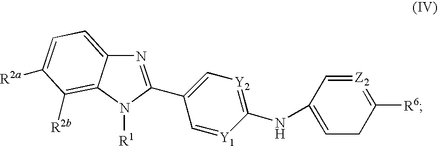
wherein
R^{2a}, R^{2b} each independently represent a group chosen from hydrogen, halogen, hydroxy, C₁₋₆alkyl, C₁₋₆halogenalkyl, C₃₋₁₂cycloalkyl, amino, C₁₋₆alkoxy, C₃₋₁₂cycloalkyloxy, sulphonate, sulphate, phosphate, quartenary ammonium, phosphonate and guanidimiumcyano;
R¹ is C₁₋₆alkyl or C₃₋₁₂cycloalkyl;
Y₁ and Y₂ each independently represent CR³ or N;
R³ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino, di(C₃₋₁₂cycloalkyl)amino or cyano;
Z₂ represents CR⁴ or N; and
R⁴ represents hydrogen, halogen, hydroxy, C₁₋₆alkyl, C₃₋₁₂cycloalkyl, C₁₋₆alkoxy, C₃₋₁₂cycloalkyloxy, amino, C₁₋₆alkylamino, C₃₋₁₂cycloalkylamino, di(C₁₋₆alkyl)amino, (C₁₋₆alkyl)(C₃₋₁₂cycloalkyl)amino or di(C₃₋₁₂cycloalkyl)amino; and
R⁶ is selected from hydrogen, hydroxy, halogen, C₁₋₆alkyl, C₃₋₁₂cycloalkyl, C₁₋₆alkoxy, C₃₋₁₂cycloalkyloxy, cyano, C₁₋₆hydroxyalkyl, C₁₋₆alkoxycarbonyl and C₁₋₆alkylcarbonylamino.

7. The compound of formula (IV) according to claim 6, wherein R¹ is C₁₋₄alkyl.

8. The compound of formula (IV) according to claim 6, wherein at least one of Y₁ and Y₂ is N.

9. The compound according to any one of the claims 1 to 8, wherein the compound is in free base or salt form.

10. A pharmaceutical composition, comprising:
a the compound according to claim 1 and a pharmaceutical carrier or diluent.

11. (canceled)

12. A method for treating disorders associated with irregularities of the glutamatergic signal transmission, the gastro-intestinal and urinary tract and nervous system disorders mediated full or in part by mGluR5, comprising:
administering to a patient in need thereof an effective amount of the compound according to claim 1.

13. The method according claim 12, wherein the disorders of the nervous system mediated full or in part by mGluR5 are selected from the group consisting of:
acute, traumatic and chronic degenerative processes of the nervous system, substance-related disorders, psychiatric diseases, affective and anxiety disorders, attention deficit disorders and cognitive dysfunction associated with these and other CNS disorders.

14. The method according claim 12, wherein the disorders of the urinary tract comprise conditions associated with pain and/or discomfort of the urinary tract and overactive bladder (OAB).

15. The method according claim 12, wherein the disorders of the gastro-intestinal tract are selected from the group consisting of: post-operative ileus, functional gastro-intestinal disorders (FGID), gastro-esophageal reflux disease (GERD), irritable bowel syndrome (IBS), functional bloating, functional diarrhoea, chronic constipation and functional disturbances of the biliary tract.

16. The method according claim 12, wherein the disorders associated with irregularities of the glutamatergic signal transmission are selected from the group consisting of:
epileptogenesis, cerebral ischemias, ischemic diseases of the eye, muscle spasms, skin disorders, obesity disorders, convulsions and pain.

17-21. (canceled)
